SEQUENCE LISTING (157 sequences)

SEQ ID NO: 1 - protein, 12 aa, Artificial Sequence
  Synthetic Construct
LLQGLLQGLLQG

SEQ ID NO: 2 - protein, 18 aa, Artificial Sequence
  Synthetic Construct
LLQGGSGLLQGGSGLLQG

SEQ ID NO: 3 - protein, 12 aa, Artificial Sequence
  Synthetic Construct
LQSPLQSPLQSP

SEQ ID NO: 4 - protein, 18 aa, Artificial Sequence
  Synthetic Construct
LQSPGSGLQSPGSGLQSP

SEQ ID NO: 5 - protein, 21 aa, Artificial Sequence
  Synthetic Construct
PNPQLPFPNPQLPFPNPQLPF

SEQ ID NO: 6 - protein, 27 aa, Artificial Sequence
  Synthetic Construct
PNPQLPFGSGPNPQLPFGSGPNPQLPF

SEQ ID NO: 7 - protein, 21 aa, Artificial Sequence
  Synthetic Construct
PKPQQFMPKPQQFMPKPQQFM

SEQ ID NO: 8 - protein, 27 aa, Artificial Sequence
  Synthetic Construct
PKPQQFMGSGPKPQQFMGSGPKPQQFM

SEQ ID NO: 9 - protein, 18 aa, Artificial Sequence
  Synthetic Construct
GQQQLGGQQQLGGQQQLG

SEQ ID NO: 10 - protein, 24 aa, Artificial Sequence
  Synthetic Construct
GQQQLGGSGGQQQLGGSGGQQQLG

SEQ ID NO: 11 - protein, 15 aa, Artificial Sequence
  Synthetic Construct
RLQQPRLQQPRLQQP

SEQ ID NO: 12 - protein, 21 aa, Artificial Sequence
  Synthetic Construct
RLQQPGSGRLQQPGSGRLQQP

SEQ ID NO: 13 - protein, 5 aa, Artificial Sequence
  Synthetic Construct
LLQGG

SEQ ID NO: 14 - protein, 4 aa, Artificial Sequence
  Synthetic Construct
LLQG

SEQ ID NO: 15 - protein, 5 aa, Artificial Sequence
  Synthetic Construct
GLLQG

SEQ ID NO: 16 - protein, 6 aa, Artificial Sequence
  Synthetic Construct
LSLSQG

SEQ ID NO: 17 - protein, 8 aa, Artificial Sequence
  Synthetic Construct
GGGLLQGG

SEQ ID NO: 18 - protein, 10 aa, Artificial Sequence
  Synthetic Construct
GSPLAQSHGG

SEQ ID NO: 19 - protein, 7 aa, Artificial Sequence
  Synthetic Construct
GLLQGGG

SEQ ID NO: 20 - protein, 6 aa, Artificial Sequence
  Synthetic Construct
GLLQGG

SEQ ID NO: 21 - protein, 4 aa, Artificial Sequence
  Synthetic Construct
GLLQ

SEQ ID NO: 22 - protein, 8 aa, Artificial Sequence
  Synthetic Construct
LLQLLQGA

SEQ ID NO: 23 - protein, 5 aa, Artificial Sequence
  Synthetic Construct
LLQGA

SEQ ID NO: 24 - protein, 7 aa, Artificial Sequence
  Synthetic Construct
LLQYQGA

SEQ ID NO: 25 - protein, 6 aa, Artificial Sequence
  Synthetic Construct
LLQGSG

SEQ ID NO: 26 - protein, 6 aa, Artificial Sequence
  Synthetic Construct
LLQYQG

SEQ ID NO: 27 - protein, 7 aa, Artificial Sequence
  Synthetic Construct
LLQLLQG

SEQ ID NO: 28 - protein, 5 aa, Artificial Sequence
  Synthetic Construct
SLLQG

SEQ ID NO: 29 - protein, 5 aa, Artificial Sequence
  Synthetic Construct
LLQLQ

SEQ ID NO: 30 - protein, 6 aa, Artificial Sequence
  Synthetic Construct
LLQLLQ

SEQ ID NO: 31 - protein, 5 aa, Artificial Sequence
  Synthetic Construct
LLQGR

SEQ ID NO: 32 - protein, 6 aa, Artificial Sequence
  Synthetic Construct
LLQGPP

SEQ ID NO: 33 - protein, 6 aa, Artificial Sequence
  Synthetic Construct
LLQGPA

SEQ ID NO: 34 - protein, 8 aa, Artificial Sequence
  Synthetic Construct
GGLLQGPP

SEQ ID NO: 35 - protein, 7 aa, Artificial Sequence
  Synthetic Construct
GGLLQGA

SEQ ID NO: 36 - protein, 7 aa, Artificial Sequence
  Synthetic Construct
LLQGPGK

SEQ ID NO: 37 - protein, 6 aa, Artificial Sequence
  Synthetic Construct
LLQGPG

SEQ ID NO: 38 - protein, 5 aa, Artificial Sequence
  Synthetic Construct
LLQGP

SEQ ID NO: 39 - protein, 4 aa, Artificial Sequence
  Synthetic Construct
LLQP

SEQ ID NO: 40 - protein, 6 aa, Artificial Sequence
  Synthetic Construct
LLQPGK

SEQ ID NO: 41 - protein, 7 aa, Artificial Sequence
  Synthetic Construct
LLQAPGK

SEQ ID NO: 42 - protein, 7 aa, Artificial Sequence
  Synthetic Construct
LLQGAPG

SEQ ID NO: 43 - protein, 6 aa, Artificial Sequence
  Synthetic Construct
LLQGAP

SEQ ID NO: 44 - protein, 6 aa, Artificial Sequence
  Synthetic Construct
LLQLQG

SEQ ID NO: 45 - protein, 6 aa, Artificial Sequence
  Synthetic Construct
QVQLKE

SEQ ID NO: 46 - protein, 5 aa, Artificial Sequence
  Synthetic Construct
VQLKE

SEQ ID NO: 47 - protein, 4 aa, Artificial Sequence
  Synthetic Construct
LQQP

SEQ ID NO: 48 - protein, 4 aa, Artificial Sequence
  Synthetic Construct
PQQF

SEQ ID NO: 49 - protein, 5 aa, Artificial Sequence
  Synthetic Construct
GQQQL

SEQ ID NO: 50 - protein, 8 aa, Artificial Sequence
  Synthetic Construct
LSGRSDNH

SEQ ID NO: 51 - protein, 5 aa, Artificial Sequence
  Synthetic Construct
SGRSA

SEQ ID NO: 52 - protein, 13 aa, Artificial Sequence
  Synthetic Construct
LGGSGRSANAILE

SEQ ID NO: 53 - protein, 13 aa, Artificial Sequence
  Synthetic Construct
LGGSGRNAQVRLE

SEQ ID NO: 54 - protein, 8 aa, Artificial Sequence
  Synthetic Construct
GSGRNAQV

SEQ ID NO: 55 - protein, 3 aa, Artificial Sequence
  Synthetic Construct
SGR

SEQ ID NO: 56 - protein, 22 aa, Artificial Sequence
  Synthetic Construct
RSQSRSRYYRQRQRSRRRRRRS

SEQ ID NO: 57 - protein, 21 aa, Artificial Sequence
  Synthetic Construct
RRRLHRIHRRQHRSCRRRKRR

SEQ ID NO: 58 - protein, 33 aa, Artificial Sequence
  Synthetic Construct
MPRRRRSSSRPVRRRRRPRVSRRRRRRGGRRRR

SEQ ID NO: 59 - protein, 28 aa, Artificial Sequence
  Synthetic Construct
KKSAKKTPKKAKKPKKSAKKTPKKAKKP

SEQ ID NO: 60 - protein, 26 aa, Artificial Sequence
  Synthetic Construct
AKKAKSPKKAKAAKPKKAPKSPAKAK

SEQ ID NO: 61 - protein, 19 aa, Artificial Sequence
  Synthetic Construct
MRRAHHRRRRASHRRMRGG

SEQ ID NO: 62 - protein, 30 aa, Artificial Sequence
  Synthetic Construct
KHKHKHKHKKKHKHKHKHKKKHKHKHKHKK

SEQ ID NO: 63 - protein, 30 aa, Artificial Sequence
  Synthetic Construct
KGKGKGKGKKKGKGKGKGKKKGKGKGKGKK

SEQ ID NO: 64 - protein, 26 aa, Artificial Sequence
  Synthetic Construct
KKALLALALHHLAHLALHLALALKKA

SEQ ID NO: 65 - protein, 22 aa, Artificial Sequence
  Synthetic Construct
YSPTSPSYSPTSPSYSPTSPSY

SEQ ID NO: 66 - DNA, 30 bp, Artificial Sequence
  Synthetic Construct
tcgaacgttcgaacgttcgaacgttcgaat

SEQ ID NO: 67 - DNA, 30 bp, Artificial Sequence; identical to SEQ ID NO: 66
  Synthetic Construct
  misc_feature (1)..(30): phosphorothioated linkages between all nucleotides

SEQ ID NO: 68 - DNA, 30 bp, Artificial Sequence; identical to SEQ ID NO: 66
  Synthetic Construct
  misc_feature 1: Modified by Amino Modifier-(CH2)12
  misc_feature (1)..(30): phosphorothioated linkages between all nucleotides

SEQ ID NO: 69 - DNA, 30 bp, Artificial Sequence; identical to SEQ ID NO: 66
  Synthetic Construct
  misc_feature 1: Modified by Amino Modifier-(CH2)12-thiol (CH2)6-S-S
  misc_feature (1)..(30): phosphorothioated linkages between all nucleotides

SEQ ID NO: 70 - DNA, 30 bp, Artificial Sequence; identical to SEQ ID NO: 66
  Synthetic Construct
  misc_feature 1: Modified by Amino Modifier-(CH2)6-(PEG)6
  misc_feature (1)..(30): phosphorothioated linkages between all nucleotides

SEQ ID NO: 71 - DNA, 30 bp, Artificial Sequence; identical to SEQ ID NO: 66
  Synthetic Construct
  misc_feature 1: Modified by Amino Modifier-(CH2)12-(PEG)6
  misc_feature (1)..(30): phosphorothioated linkages between all nucleotides

SEQ ID NO: 72 - DNA, 30 bp, Artificial Sequence; identical to SEQ ID NO: 66
  Synthetic Construct
  misc_feature 1: Modified by Thiol Modifier-(CH2)6-S-S
  misc_feature (1)..(30): phosphorothioated linkages between all nucleotides

SEQ ID NO: 73 - DNA, 30 bp, Artificial Sequence; identical to SEQ ID NO: 66
  Synthetic Construct
  misc_feature (1)..(30): phosphorothioated linkages between all nucleotides
  misc_feature 30: Modified by S-S-(CH2)6-Thiol Modifier

SEQ ID NO: 74 - protein, 35 aa, Artificial Sequence
  Synthetic Construct
  VARIANT 13,14: positions 13 and 14 are linked by a (PEG)12 moiety
DCAWHLGELVWCTRSQSRSRYYRQRQRSRRRRRRS

SEQ ID NO: 75 - protein, 13 aa, Artificial Sequence
  Synthetic Construct
DCAWHLGELVWCT

SEQ ID NO: 76 - protein, 13 aa, Artificial Sequence
  Synthetic Construct
DCAWHKGELVWCT

SEQ ID NO: 77 - protein, 13 aa, Artificial Sequence
  Synthetic Construct
NCAWHKGQLVWCT

SEQ ID NO: 78 - protein, 18 aa, Artificial Sequence
  Synthetic Construct
GSKGSDCAWHLGELVWCT

SEQ ID NO: 79 - protein, 18 aa, Artificial Sequence
  Synthetic Construct
GSKGSNCAWHLGQLVWCT

SEQ ID NO: 80 - protein, 35 aa, Artificial Sequence
  Synthetic Construct
  VARIANT 13,14: positions 13 and 14 are linked by a (PEG)12 moiety
DCAWHLGQLVWCTRSQSRSRYYRQRQRSRRRRRRS

SEQ ID NO: 81 - protein, 40 aa, Artificial Sequence
  Synthetic Construct
  VARIANT 18,19: positions 18 and 19 are linked by a (PEG)12 moiety
GSKGSDCAWHLGELVWCTRSQSRSRYYRQRQRSRRRRRRS

SEQ ID NO: 82 - protein, 40 aa, Artificial Sequence
  Synthetic Construct
  VARIANT 18,19: positions 18 and 19 are linked by a (PEG)12 moiety
GSCGSDCAWHLGELVWCTRSQSRSRYYRQRQRSRRRRRRS

SEQ ID NO: 83 - protein, 40 aa, Artificial Sequence
  Synthetic Construct
  VARIANT 18,19: positions 18 and 19 are linked by a (PEG)12 moiety
GSDGSNCAWHLGQLVWCTRSQSRSRYYRQRQRSRRRRRRS

SEQ ID NO: 84 - protein, 451 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 215 aa, Artificial Sequence
  Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 5 aa, Artificial Sequence
  Synthetic Construct
RYWMS

SEQ ID NO: 87 - protein, 17 aa, Artificial Sequence
  Synthetic Construct
NIKQDGSEKYYVDSVKG

SEQ ID NO: 88 - protein, 12 aa, Artificial Sequence
  Synthetic Construct
EGGWFGELAFDY

SEQ ID NO: 89 - protein, 12 aa, Artificial Sequence
  Synthetic Construct
RASQRVSSSYLA

SEQ ID NO: 90 - protein, 7 aa, Artificial Sequence
  Synthetic Construct
DASSRAT

SEQ ID NO: 91 - protein, 9 aa, Artificial Sequence
  Synthetic Construct
QQYGSLPWT

SEQ ID NO: 92 - protein, 448 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 5 aa, Artificial Sequence
  Synthetic Construct
DSWIH

SEQ ID NO: 95 - protein, 17 aa, Artificial Sequence
  Synthetic Construct
WISPYGGSTYYADSVKG

SEQ ID NO: 96 - protein, 9 aa, Artificial Sequence
  Synthetic Construct
RHWPGGFDY

SEQ ID NO: 97 - protein, 12 aa, Artificial Sequence
  Synthetic Construct
RASQRVSSSYLA

SEQ ID NO: 98 - protein, 7 aa, Artificial Sequence
  Synthetic Construct
DASSRAT

SEQ ID NO: 99 - protein, 9 aa, Artificial Sequence
  Synthetic Construct
QQYGSLPWT

SEQ ID NO: 100 - protein, 525 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPLQSPLQSPGGGGSGGGGSGSGSGRSAGSGGGGGSGGGSRSQSRSRYYRQRQRSRRRRRRS

SEQ ID NO: 101 - protein, 215 aa, Artificial Sequence; identical to SEQ ID NO: 85
  Synthetic Construct

SEQ ID NO: 102 - protein, 451 aa, Artificial Sequence; identical to SEQ ID NO: 84
  Synthetic Construct

SEQ ID NO: 103 - protein, 215 aa, Artificial Sequence; identical to SEQ ID NO: 85
  Synthetic Construct

SEQ ID NO: 104 - protein, 482 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPGSGLQSPGSGLQSPGSG

SEQ ID NO: 105 - protein, 215 aa, Artificial Sequence; identical to SEQ ID NO: 85
  Synthetic Construct

SEQ ID NO: 106 - protein, 555 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPGSGLQSPGSGLQSPGGGGSGGGGSLGGSGRNAQVRLEGGGGSGGGSKGKGKGKGKKMPRRRRSSSRPVRRRRRPRVSRRRRRRGGRRRRS

SEQ ID NO: 107 - protein, 215 aa, Artificial Sequence; identical to SEQ ID NO: 85
  Synthetic Construct

SEQ ID NO: 108 - protein, 538 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPGSGLQSPGSGLQSPGGGGSGGGGSLGGSGRNAQVRLEGGGGSGGGSAKKAKSPKKAKAAKPKKAPKSPAKAKS

SEQ ID NO: 109 - protein, 215 aa, Artificial Sequence; identical to SEQ ID NO: 85
  Synthetic Construct

SEQ ID NO: 110 - protein, 542 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPGSGLQSPGSGLQSPGGGGSGGGGSLGGSGRNAQVRLEGGGGSGGGSKGKGKGKGKKKGKGKGKGKKKGKGKGKGKKS

SEQ ID NO: 111 - protein, 215 aa, Artificial Sequence; identical to SEQ ID NO: 85
  Synthetic Construct

SEQ ID NO: 112 - protein, 505 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLGGSGRNAQVRLEGGGGSGGGGSLQSPGSGLQSPGSGLQSPGSG

SEQ ID NO: 113 - protein, 215 aa, Artificial Sequence; identical to SEQ ID NO: 85
  Synthetic Construct

SEQ ID NO: 114 - protein, 992 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLQSPGSGLQSPGSGLQSPGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSGGGGSGGGGSGGGGSGGGGSGGGGSGGGGSGGGGSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, Artificial Sequence; identical to SEQ ID NO: 85
  Synthetic Construct

SEQ ID NO: 116 - protein, 1015 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSLGGSGRNAQVRLEGGGGSGGGGSLQSPGSGLQSPGSGLQSPGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSGGGGSGGGGSGGGGSGGGGSGGGGSGGGGSGGGGSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, Artificial Sequence; identical to SEQ ID NO: 85
  Synthetic Construct

SEQ ID NO: 118 - protein, 709 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAA

SEQ ID NO: 119 - protein, 215 aa, Artificial Sequence; identical to SEQ ID NO: 85
  Synthetic Construct

SEQ ID NO: 120 - protein, 705 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 121 - protein, 215 aa, Artificial Sequence; identical to SEQ ID NO: 85
  Synthetic Construct

SEQ ID NO: 122 - protein, 734 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGLQSPGSGLQSPGSGLQSPGSGGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 123 - protein, 215 aa, Artificial Sequence; identical to SEQ ID NO: 85
  Synthetic Construct

SEQ ID NO: 124 - protein, 752 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSLGGSGRNAQVRLEGGGGSGSGLQSPGSGLQSPGSGLQSPGSGGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 125 - protein, 215 aa, Artificial Sequence; identical to SEQ ID NO: 85
  Synthetic Construct

SEQ ID NO: 126 - protein, 705 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGCGTKVEIKRGGGGSGGGGSGGGGSQVQLQQSGSELKKPGASVKVSCKASGYTFTNYGMNWVKQAPGQCLKWMGWINTYTGEPTYTDDFKGRFAFSLDTSVSTAYLQISSLKADDTAVYFCARGGFGSSYWYFDVWGQGSLVTVSS

SEQ ID NO: 127 - protein, 215 aa, Artificial Sequence; identical to SEQ ID NO: 85
  Synthetic Construct

SEQ ID NO: 128 - protein, 734 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGSGLQSPGSGLQSPGSGLQSPGSGGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGGGTKLTVLGGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 129 - protein, 215 aa, Artificial Sequence; identical to SEQ ID NO: 85
  Synthetic Construct

SEQ ID NO: 130 - protein, 503 aa, Artificial Sequence
  Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSGGGGSGGGGSGGGGSGGGGSGGGGSGGGGSGGGGSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 248 aa, Artificial Sequence
  Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAA

SEQ ID NO: 132 - protein, 244 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 133 - protein, 244 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGGGTKLTVLGGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 134 - protein, 5 aa, Artificial Sequence
  Synthetic Construct
NYGMN

SEQ ID NO: 135 - protein, 17 aa, Artificial Sequence
  Synthetic Construct
WINTYTGEPTYTDDFKG

SEQ ID NO: 136 - protein, 12 aa, Artificial Sequence
  Synthetic Construct
GGFGSSYWYFDV

SEQ ID NO: 137 - protein, 11 aa, Artificial Sequence
  Synthetic Construct
KASQDVSIAVA

SEQ ID NO: 138 - protein, 7 aa, Artificial Sequence
  Synthetic Construct
SASYRYT

SEQ ID NO: 139 - protein, 9 aa, Artificial Sequence
  Synthetic Construct
QQHYITPLT

SEQ ID NO: 140 - protein, 121 aa, Artificial Sequence
  Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 141 - protein, 112 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAA

SEQ ID NO: 142 - protein, 108 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 143 - protein, 107 aa, Artificial Sequence
  Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGGGTKLTVL

SEQ ID NO: 144 - protein, 10 aa, Artificial Sequence
  Synthetic Construct
HHHHHHGGGS

SEQ ID NO: 145 - protein, 4 aa, Artificial Sequence
  Synthetic Construct
  VARIANT 1, 2, 4: Xaa = Any Amino Acid
XXQX

SEQ ID NO: 146 - protein, 13 aa, Artificial Sequence
  Synthetic Construct
LGGSGRNAQVRLE

SEQ ID NO: 147 - protein, 16 aa, Artificial Sequence
  Synthetic Construct
  VARIANT 1: Modified by an Amino Modifier
  VARIANT 16: Linked by a (PEG)12 moiety to position 1 in SEQ ID NO: 157
LGGSGRNAQVRLEGSG

SEQ ID NO: 148 - protein, 56 aa, Artificial Sequence
  Synthetic Construct
  VARIANT 18,19: positions 18 and 19 are linked by a (PEG)12 moiety
GSKGSDCAWHLGELVWCTLGGSGRNAQVRLEGSGRSQSRSRYYRQRQRSRRRRRRS

SEQ ID NO: 149 - protein, 56 aa, Artificial Sequence
  Synthetic Construct
  VARIANT 18,19: positions 18 and 19 are linked by a (PEG)12 moiety
GSCGSDCAWHLGELVWCTLGGSGRNAQVRLEGSGRSQSRSRYYRQRQRSRRRRRRS

SEQ ID NO: 150 - protein, 53 aa, Artificial Sequence
  Synthetic Construct
  VARIANT 18,19: positions 18 and 19 are linked by a (PEG)12 moiety
GSDGSNCAWHLGQLVWCTLGGSGRNAQVRLERSQSRSRYYRQRQRSRRRRRRS

SEQ ID NO: 151 - protein, 22 aa, Artificial Sequence
  Synthetic Construct
  VARIANT 1: Modified by NH2-(PEG)12 moiety
RSQSRSRYYRQRQRSRRRRRRS

SEQ ID NO: 152 - protein, 13 aa, Artificial Sequence
  Synthetic Construct
DCAWHLGQLVWCT

SEQ ID NO: 153 - protein, 4 aa, Artificial Sequence
  Synthetic Construct
LQSP

SEQ ID NO: 154 - protein, 3 aa, Artificial Sequence
  Synthetic Construct
  VARIANT 1: Xaa = Any Amino Acid, and can be absent or present in any number of repeats
  VARIANT 2: Can be present in any number of repeats
  VARIANT 3: Xaa = Any Amino Acid, and can be absent or present in any number of repeats
  VARIANT 3: Modified by either zero or one linker
  VARIANT (1)..(3): Including the linker linked to the amino acid at position 3, can be present in any number of repeats
XQX

SEQ ID NO: 155 - protein, 4 aa, Artificial Sequence
  Synthetic Construct
  VARIANT 1, 3, 4: Xaa = Any Amino Acid
  VARIANT 4: Modified by either zero or one amino acid linker
  VARIANT (1)..(4): Including the linker linked to the amino acid at position 4, can be present in any number of repeats
XQXX

SEQ ID NO: 156 - skipped

SEQ ID NO: 157 - DNA, 30 bp, Artificial Sequence; identical to SEQ ID NO: 66
  Synthetic Construct
  misc_feature (1)..(30): phosphorothioated linkages between all nucleotides
  misc_feature 1: Linked by a (PEG)12 moiety to position 16 in SEQ ID NO: 147

(19) United States
(12) Patent Application Publication
(10) Pub. No.: US 2024/0299576 A1
(43) Pub. Date: Sep. 12, 2024

(54) TARGETING CONJUGATES COMPRISING EFFECTOR MOLECULES AND USES THEREOF

(71) Applicant: Allygen Group, Grand Cayman (KY)

(72) Inventors: Jun XIANG, Shanghai (CN); Wenlong GAO, Shanghai (CN)

(21) Appl. No.: 18/278,787

(22) PCT Filed: Feb. 24, 2022
(86) PCT No.: PCT/CN2022/077724

§ 371 Date: Aug. 24, 2023

(30) Foreign Application Priority Data
Feb. 25, 2021 (WO) PCT/CN2021/077864
Apr. 1, 2021 (WO) PCT/CN2021/084952

(51) Int. Cl. A61K 47/68

(52) U.S. Cl. CPC A61K 47/6889; A61K 47/6879

(57) ABSTRACT

Provided are targeting conjugates comprising a targeting moiety and an effector molecule, wherein the effector molecule is conjugated to the targeting moiety via a conjugation site, wherein the effector molecule can be released from the targeting conjugate via cleavage. Also provided are compositions and methods of treatment or diagnosis using the targeting conjugates.

### CROSS-REFERENCE TO RELATED APPLICATIONS

The application claims priority to PCT application PCT/CN2021/077864, filed Feb. 25, 2021, and PCT application PCT/CN2021/084952, filed Apr. 1, 2021, the contents of both of which are incorporated herein by reference in their entireties.

### FIELD

The present application relates to targeting conjugates comprising effector molecules and uses thereof.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 199872000240SEQLIST.TXT, date recorded: Feb. 24, 2021, size: 163 KB).

### BACKGROUND

Antibodies have been successfully used as therapeutic and diagnostic agents for a variety of diseases. A native antibody acts by binding to a specific target. However, antibody targets can be expressed in both diseased (e.g. tumor) tissues and normal tissues. When antibodies bind to targets expressed in normal tissues of a patient, the patient can experience undesirable, toxic side-effects, which can range from mild to severe or even life-threatening degree of intensity. Such side effects can lead to a decreased effective dosage of the antibody, which can lower its therapeutic efficacy. The antibody treatment may even be discontinued due to the side effects, which, in some cases, render the antibody therapeutic unavailable to human patients. Thus, there exists an unmet need to engineer antibodies that preferentially bind to targets on diseased cells and tissues.

Antibody drug conjugates (ADCs) are targeted therapeutics designed to preferentially direct drugs (“payloads”) to diseased tissues expressing a surface antigen recognized by the antibody. ADCs are often composed of an antibody linked to therapeutically active agents (e.g. a cytotoxic drug), via chemical linkers that enable release of therapeutically active agents in the diseased environment. Currently, five ADCs have been approved by the FDA for therapeutic use, and over 100 are in clinical investigation. Trastuzumab emtansine (KADCYLA®) and brentuzimab vedotin (ADCETRIS®) are among the most widely used ADCs in cancer treatment (see, for example, Jackson et al., Pharm Res (2015) 32:3458-3469). The majority of the ADCs currently approved or undergoing clinical evaluation are small molecule drug-antibody conjugates. Current ADCs incorporate standard chemotherapeutics such as antimitotics and antimetabolites, including auristatins monomethyl auristatin E (MMAE), calicheamicin, and derivative of maytansin 1 (DM1). See, for example, Polakis, Pharma Rev, 2016, 68(1)3-19.

Although ADCs have conceptual advantages and promising clinical results, the development of an effective ADC therapeutic remains remarkably challenging. The overall design of the ADC, the choice of target tissues, the antibody, the chemical linker, the site of drug attachment, and the nature of the cargo all influence the efficacy and the risks of the ADC (see, for example, Chau et al., Lancet 2019; 394:793-804). For instance, early ADCs in clinical trials suffer from immunogenicity of the mouse antibodies used, and recent advances in the development of ADCs rely on humanized antibodies. Other outstanding challenges in ADCs include a) drug cargo impotency; b) premature release of drug leading to loss of efficacy and toxicity; c) level of target expression; d) suboptimal target selectivity; e) side effects such as thrombocytopenia and neuropathy as seen in KADCYLA® and ADCETRIS®. These limitations underscore the need for further improvement of ADCs in search of high potency payloads, stable chemical linkers, efficient and highly specific cargo release, and increased antibody-target selectivity.

The ADC platform as a highly specific cargo delivery system in vivo also lends itself to applications beyond small molecule drug delivery. For example, siRNAs can be fused to an antibody and developed into an efficient method for in vivo mRNA knockdown (see, Baumer et al., Nature Protocols, 2016, 11:22-36). Such antibody-mediated siRNA delivery has shown promising results in colon cancer treatment (see, Baumer et al., Clin Cancer Res 2015 15; 21(6):1383-94.). There remains a need for a broader scope in the design, types of antibodies, and breadth of therapeutic agents in ADC platforms.

### BRIEF SUMMARY

The present application provides targeting conjugates comprising a targeting moiety and one or more effector molecules, compositions, methods of preparation and methods of use thereof.

In some embodiments, there is provided a targeting conjugate comprising a targeting moiety and an effector molecule, wherein the effector molecule is conjugated to the targeting moiety via a conjugation site, wherein the effector molecule can be released from the targeting conjugate via cleavage. In some embodiments, the targeting conjugate comprises the structure of Formula I:

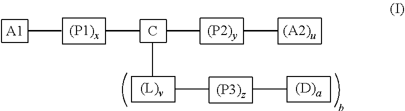

wherein: A1 is a first targeting moiety; A2 is a second targeting moiety; P1 is a first cleavage site; P2 is a second cleavage site; P3 is a third cleavage site; C is the conjugation site; L is a linker; D is the effector molecule; x=0 or 1; y=0 or 1; z=0 or 1; u=0 or 1; v=0 or 1; a=1-20; and b=1-20. In some embodiments, z=0. In some embodiments, z=1. In some embodiments, y=0. In some embodiments, y=1. In some embodiments, x=0. In some embodiments, x=1. In some embodiments, u=0. In some embodiments, u=1. In some embodiments, a is 1 or more. In some embodiments, b is 1 or more.

In some embodiments according to any one of the targeting conjugates described above, the targeting conjugate comprises a targeting peptide. In some embodiments, the targeting conjugate comprises an antibody or antigen-binding fragment thereof. In some embodiments, the antibody or antigen-binding fragment thereof is selected from the group consisting of: a human antibody, a humanized antibody, a chimeric antibody, a monospecific antibody, a multispecific antibody, a diabody, a nanobody, an scFv, an scFab, a Fab fragment, a Fab′ fragment, a F(ab′)₂ fragment, and a dsFv. In some embodiments, the targeting moiety comprises an antibody-peptide fusion protein. In some embodiments, the antibody-peptide fusion protein comprises an Fc region. In some embodiments, the peptide is fused to the C-terminus of the Fc region.

In some embodiments according to any one of the targeting conjugates described above, the targeting conjugate comprises a targeting moiety that specifically binds to a target molecule (e.g., cell surface molecule) at a target site. In some embodiments, the target site is a site of a disease. In some embodiments, the disease is tumor. In some embodiments, the target molecule is selected from the group consisting of: PD1, PD-L1, Trop2, CTLA-4, LAG-3, TIM-3, 4-1BB, CD40, OX40, CD47, SIRPα, HER2, HER3, EGFR, VEGF, VEGR2, CD19, CD20, CD22, CD30, CD33, CD38, CD79, integrin αvβ, αvβ6, MUC1, PMSA, uPAR, and angiopep-2.

In some embodiments according to any one of the targeting conjugates described above, the cleavage is triggered by a condition at a target site for the targeting moiety. In some embodiments, the condition at a target site is selected from the group consisting of: protease, pH change, redox change, hypoxia, oxidative stress, hyperthermia, and extracellular ATP concentration. In some embodiments, the target site is a site of a disease. In some embodiments, the disease is tumor.

In some embodiments according to any one of the targeting conjugates described above, the cleavage is by a protease. In some embodiments, the protease is selected from the group consisting of: urokinase plasminogen activator (uPA), legumain, plasmin, TMPRSS3, TMPRSS4, TMPRSS6, MMP1, MMP2, MMP-3, MMP-9, MMP-8, MMP-14, MT1-MMP, CATHEPSIN D, CATHEPSIN K, CATHEPSIN S, ADAM 10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, TACE, human neutrophil elastase, beta-secretase, fibroblast associated protein, matriptase, PSMA and PSA. In some embodiments, the protease is uPA. In some embodiments, the cleavage site comprises an amino acid sequence selected from the amino acid sequences of SEQ ID NOs: 50-55.

In some embodiments according to any one of the targeting conjugates described above, the effector molecule is selected from the group consisting of a therapeutic agent, an oligonucleotide, and a detectable label. In some embodiments, the effector molecule is a therapeutic agent. In some embodiments, the effector molecule is selected from the group consisting of a protein-based drug, a small molecule drug, a cytotoxic agent, a toxin, an immunomodulatory agent, an anti-inflammatory agent, an anti-infective agent, and an epigenetic modulating agent. In some embodiments, the effector molecule is an oligonucleotide. In some embodiments, the oligonucleotide is about 2 to about 100 nucleotides long. In some embodiments, the oligonucleotide is selected from the group consisting of: a double stranded DNA, a single stranded DNA, a double stranded RNA, a single stranded RNA, an antisense RNA, a small interference RNA, a microRNA, a short hairpin RNA (shRNA), and a CpG (Cytosine-phosphodiester-Guanine) oligonucleotide. In some embodiments, the oligonucleotide is a CpG oligonucleotide. In some embodiments, the oligonucleotide comprises a nucleic acid sequence selected from the group consisting of SEQ ID NOs: 66-67.

In some embodiments according to any one of the targeting conjugates described above, the targeting conjugate comprises two or more effector molecules.

In some embodiments according to any one of the targeting conjugates described above, the effector molecule is a cytotoxic agent selected from the group consisting of an anthracycline, an auristatin, a camptothecin, a combretastatin, a dolastatin, a duocarmycin, an enediyne, a geldanamycin, an indolino-benzodiazepine dimer, a maytansine, a puromycin, a pyrrolobenzodiazepine dimer, a taxane, a vinca alkaloid, a tubulysin, a hemiasterlin, a spliceostatin, a pladienolide, and stereoisomers, isosteres, analogs, or derivatives thereof. In some embodiments, the effector molecule is SN38 or a derivative thereof. In some embodiments, the effector molecule is MMAE or a derivative thereof. In some embodiments, the effector molecule is selected from compounds of Formulae (1)-(7). In some embodiments, the target conjugate comprises two or more small molecule effector molecules.

In some embodiments according to any one of the targeting conjugates described above, wherein the targeting conjugate comprises the structure of Formula I, A1 is a first targeting peptide, or a first antibody or antigen-binding fragment thereof recognizing a first target molecule, and A2 is a second targeting peptide, or a second antibody or antigen-binding fragment thereof recognizing a second target molecule. In some embodiments, the first target molecule and the second target molecule are the same. In some embodiments, the first target molecule and the second target molecule are different. In some embodiments, A1 is connected to the N-terminus of the conjugation site (C), and wherein A2 is connected to the C-terminus of the conjugation site (C).

In some embodiments according to any one of the targeting conjugates described above, the conjugation site is a covalent conjugation site. In some embodiments, the conjugation site is an endogenous conjugation site. In some embodiments, the conjugation site is an engineered conjugation site introduced into the targeting moiety. In some embodiments, the conjugation site is present in a peptide fused to the targeting moiety. In some embodiments, the conjugation site is a transglutaminase conjugation site, such as a glutamine-containing tag. In some embodiments, the conjugation site comprises a plurality of glutamine-containing tags that are fused to each other in tandem. In some embodiments, the conjugation site comprises an amino acid sequence selected from the group consisting of SEQ ID NOs: 1-12.

In some embodiments according to any one of the targeting conjugates described above, the conjugation site is a non-covalent conjugation site. In some embodiments, the conjugation site comprises an oligonucleotide binding polypeptide. In some embodiments, the conjugation site comprises a CpG binding polypeptide. In some embodiments, the conjugation site comprises an amino acid sequence selected from the group consisting of SEQ ID NOs: 56-65.

In some embodiments according to any one of the targeting conjugates described above, wherein the targeting conjugate comprises the structure of Formula I, L is represented by the formula: SH₂-Spacer, MAL-Spacer, NH₂-Spacer, or Osu-Spacer (Osu: oxysuccinimide). In some embodiments, L is represented by the formula: (Gly)ₙ-(PEG)ₘ-VC-PAB-(DMAE)ₖ (Formula II), wherein n≥1, m≥2, and k=0 or 1. In some embodiments, L is represented by the formula: (Gly)ₙ-(PEG)ₘ-Val-Ala-PAB-(DMAE)ₖ, wherein n≥1, m≥2, and k=0 or 1. In some embodiments, L is represented by (Gly)ₙ-(PEG)ₘ-P-PAB-(DMAE)ₖ (Formula III), wherein n, m and k are integers, n≥1, m≥2, and k=0 or 1, P is a cleavage site.

In some embodiments, there is provided a targeting conjugate comprising a targeting moiety conjugated to a therapeutic agent having a formula selected from the group consisting of compounds of Formulae (1)-(7).

Also described herein is a composition comprising a plurality of any one of the targeting conjugates described above. In some embodiments, the average ratio of the effector molecule and the targeting moiety in the composition is at least about 1:1. In some embodiments, at least two of the targeting conjugates in the composition comprise different numbers of effector molecules. In some embodiments, wherein the effector molecule is a therapeutic agent or an oligonucleotide, the composition is a pharmaceutical composition. In some embodiments, wherein the effector molecule is a detectable label, the composition is a diagnostic composition.

In some embodiments, there is provided a method of treating a disease in an individual, comprising administering to the individual an effective amount of any one of the pharmaceutical compositions described above.

In some embodiments, there is provided a method of diagnosing a disease in an individual, comprising administering to the individual an effective amount of the diagnostic compositions described above, wherein the detection of the detectable label is indicative of the presence of the disease.

Also provided herein is a method of making any one of the targeting conjugates describe above, wherein the method comprises conjugating the effector molecule to the targeting moiety.

### BRIEF DESCRIPTION OF THE DRAWINGS

FIGS. 1A-10C depicts a schematic structure of an exemplary targeting conjugate, comprising A1: a first targeting moiety, such as an antibody or antigen-binding fragment or a targeting peptide (including a multispecific antibody and a fusion protein) with or without protease cleavage sites; A2: a second targeting moiety, such as an antibody or antigen-binding fragment or a targeting peptide (including a multispecific antibody and a fusion protein) with or without protease cleavage sites; D: effector molecule, such as a small molecule or an oligonucleotide; P1: Protease cleavage site 1; P2: Protease cleavage site 2; P3: Protease cleavage site 3; C: Conjugation site, such as a covalent conjugation site, or a non-covalent conjugation site (e.g., a cationic peptide or neutral-charge peptide); L: linker; x=0 or 1; y=0 or 1; z=0 or 1; u=0 or 1; v=0 or 1; a=1-20; b=1-20.

FIG. 1A shows an exemplary targeting conjugate, in which x=1; y=0; z=0; u=0; v=1; a=1-20; b=1-20.

FIG. 1B shows an exemplary targeting conjugate, in which x=1; y=0; z=0; u=1; v=1; a=1-20; b=1-20.

FIG. 1C shows an exemplary targeting conjugate, in which x=1; y=1; z=0; u=1; v=1; a=1-20; b=1-20.

FIG. 2A shows an exemplary targeting conjugate, in which x=1; y=0; z=1; u=0; v=1; a=1-20; b=1-20.

FIG. 2B shows an exemplary targeting conjugate, in which x=1; y=0; z=1; u=1; v=1; a=1-20; b=1-20.

FIG. 2C shows an exemplary targeting conjugate, in which x=1; y=1; z=1; u=1; v=1; a=1-20; b=1-20.

FIG. 3A shows an exemplary targeting conjugate, in which x=0; y=0; z=0; u=0; v=1; a=1-20; b=1-20.

FIG. 3B shows an exemplary targeting conjugate, in which x=0; y=0; z=0; u=1; v=1a=1-20; b=1-20.

FIG. 3C shows an exemplary targeting conjugate, in which x=0; y=1; z=0; u=1; v=1; a=1-20; b=1-20.

FIG. 4A shows an exemplary targeting conjugate, in which x=0; y=0; z=1; u=0; v=1; a=1-20; b=1-20.

FIG. 4B shows an exemplary targeting conjugate, in which x=0; y=0; z=1; u=1; v=1; a=1-20; b=1-20.

FIG. 4C shows an exemplary targeting conjugate, in which x=0; y=1; z=1; u=1; v=1; a=1-20; b=1-20.

FIG. 5A shows an exemplary targeting conjugate, in which x=1; y=0; z=0; u=0; v=1; a=1-20; b=1-20.

FIG. 5B shows an exemplary targeting conjugate, in which x=1; y=0; z=0; u=1; v=1; a=1-20; b=1-20.

FIG. 5C shows an exemplary targeting conjugate, in which x=1; y=1; z=0; u=1; v=1; a=1-20; b=1-20.

FIG. 6A shows an exemplary targeting conjugate, in which x=1; y=0; z=1; u=0; v=1; a=1-20; b=1-20.

FIG. 6B shows an exemplary targeting conjugate, in which x=1; y=0; z=1; u=1; v=1; a=1-20; b=1-20.

FIG. 6C shows an exemplary targeting conjugate, in which x=1; y=1; z=1; u=1; v=1; a=1-20; b=1-20.

FIG. 7A shows an exemplary targeting conjugate, in which x=0; y=0; z=0; u=0; v=1; a=1-20; b=1-20.

FIG. 7B shows an exemplary targeting conjugate, in which x=0; y=0; z=0; u=1; v=1; a=1-20; b=1-20.

FIG. 7C shows an exemplary targeting conjugate, in which x=0; y=1; z=0; u=1; v=1; a=1-20; b=1-20.

FIG. 8A shows an exemplary targeting conjugate, in which x=0; y=0; z=1; u=0; v=1; a=1-20; b=1-20.

FIG. 8B shows an exemplary targeting conjugate, in which x=0; y=0; z=1; u=1; v=1; a=1-20; b=1-20.

FIG. 8C shows an exemplary targeting conjugate, in which x=0; y=1; z=1; u=1; v=1; a=1-20; b=1-20.

FIG. 9A shows an exemplary targeting conjugate, in which x=1; y=0; z=0; u=0; v=0; a=1-20; b=1-20.

FIG. 9B shows an exemplary targeting conjugate, in which x=1; y=0; z=0; u=1; v=0; a=1-20; b=1-20.

FIG. 9C shows an exemplary targeting conjugate, in which x=1; y=1; z=0; u=1; v=0; a=1-20; b=1-20.

FIG. 10A shows an exemplary targeting conjugate, in which x=0; y=0; z=0; u=0; v=0; a=1-20; b=1-20.

FIG. 10B shows an exemplary targeting conjugate, in which x=0; y=0; z=0; u=1; v=0; a=1-20; b=1-20.

FIG. 10C shows an exemplary targeting conjugate, in which x=0; y=1; z=0; u=1; v=0; a=1-20; b=1-20.

FIG. 11 shows the gel shift assay for CpG ODN linkers non-covalently conjugated to antibodies.

FIG. 12 shows the gel shift assay for CpG ODN linkers covalently conjugated to antibodies.

FIG. 13 shows the gel shift assay for CpG ODN linker covalently conjugated to antibodies.

FIG. 14 shows the dose response curves of PD-1/PD-L1 Immunoblockade reporter assay.

FIG. 15A shows the dose response cytotoxicity curves of compounds using HCT15-hPDL1 cells.

FIG. 15B shows the dose response cytotoxicity curves of antibodies using HCT15-hPDL1 cells.

FIG. 15C shows the response cytotoxicity curves of ADCs using HCT15-hPDL1 cells.

FIG. 16 shows the in vivo anti-tumor activity of the antibodies and conjugates in MDA-MB-231 CDX model.

FIG. 17 shows the dose response cytotoxicity curves of drugs using PC3-hPDL1-hTROP2 cells.

FIG. 18 shows the dose response cytotoxicity curves of ADCs using PC3-hPDL1-hTROP2 cells.

FIG. 19 shows the dose response cytotoxicity curves of drugs using HCT15-hPDL1-hTROP2 cells.

FIG. 20 shows the in vivo anti-tumor activity in MC38-hPDL1-hTROP2 mouse syngeneic tumor model.

FIG. 21 shows the in vivo anti-tumor activity in PBMCs-PC3-hPDL1-hTROP2 CDX model.

FIG. 22 shows the in vivo anti-tumor activity in HCT15-hPDL1-hTROP2 CDX model.

FIG. 23 shows the response of HEK-Blue hTLR9-hPDL1 cells to different CpG ODN-Linkers.

FIG. 24 shows the response of HEK-Blue hTLR9-hPDL1 cells to AOC.

FIG. 25 shows in vivo anti-tumor activity in MC38-hPDL1 mouse syngeneic tumor model.

### DETAILED DESCRIPTION

The present application provides compositions and methods of treatment or diagnosis using targeting conjugates comprising a targeting moiety and an effector molecule, wherein the effector molecule is conjugated to the targeting moiety via a conjugation site, wherein the effector molecule can be released from the targeting conjugate via cleavage. The targeting moiety specifically recognizes and binds to a target, such as a cell surface molecule that is expressed on a diseased tissue or diseased cells (e.g., tumor cells). Upon binding of the targeting moiety to the target, cleavage of one or more cleavage sites in the targeting conjugate may be triggered, which lead to release of the effector molecule at the target site. In some embodiments, the targeting conjugate is multispecific. In some embodiments, the targeting conjugate comprises a first targeting moiety and a second targeting moiety, wherein the first targeting moiety and the second targeting moiety are fused to each other via a conjugation site to which one or more effector molecules are conjugated. For example, a targeting moiety can be designed by fusing one or more scFv or scFab to a monoclonal antibody (e.g., anti-PDL1 mAb or anti-Trop2 mAb) with proven clinical efficacy and safety to provide multispecificity in target recognition to provide enhanced clinical benefits. The targeting conjugates described herein may have more than one type of cargo (e.g., oligonucleotides and small molecule drugs) conjugated to the conjugation site, and the targeting moiety can be a targeting peptide, or an antibody or antigen binding fragment thereof (including a multispecific antibody), or a combination thereof. Thus, the targeting conjugates described herein can have enhanced selective target recognition capability and efficacy compared to traditional ADCs. Incorporating multiple conjugation sites and different types of cargos onto one targeting conjugate opens up potentials for more potent therapeutic effects. The possibilities of combinations of oligonucleotides and other therapeutic agents (e.g., small molecule drugs) expand the scope of diseases that can be targeted by the targeting conjugates described herein.

Accordingly, one aspect of the present application provides a targeting conjugate comprising a targeting moiety and an effector molecule, wherein the effector molecule is conjugated to the targeting moiety via a conjugation site, wherein the effector molecule can be released from the targeting conjugate via cleavage. In some embodiments, the cleavage occurs outside of a cell. In some embodiments, the targeting conjugate comprises the structure of Formula (I), wherein x=0 or 1; y=0 or 1; z=0 or 1; u=0 or 1; v=0 or 1; a=1-20; and b=1-20.

Also provided are compositions, kits and articles of manufacture for use in any one the methods described above.

### I. Definitions

Terms are used herein as generally used in the art, unless otherwise defined as follows.

The term “targeting moiety” as used herein refers to a polypeptide-based binding molecule that specifically binds to a target molecule, or a portion thereof that contributes to the specific binding. Both antibody-based and non-antibody based binding molecules or portions thereof are contemplated herein.

The term “therapeutic agent” as used herein refers to a molecule having a therapeutic effect. The therapeutic agent may be of any suitable molecular entity, except for oligonucleotides.

The term “effector molecule” as used herein refers to a molecule that may be used for therapeutic and/or diagnostic purposes. Exemplary effector molecules include, but are not limited to, therapeutic agents, diagnostic labels and oligonucleotides.

The term “conjugation” refers to chemically joining two chemical groups or moieties together via one or more covalent or non-covalent bonds. Conjugation may be direct between the two chemical groups or moieties, or indirect through a third chemical group or moiety (e.g., a linker) that bridges the two chemical groups or moieties.

The term “conjugation site” refers to a site that directly joins two chemical moieties.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof. Antibodies and/or antigen binding fragments may be derived from murine antibodies, rabbit antibodies, human antibodies, fully humanized antibodies, camelid antibody variable domains and humanized versions, shark antibody variable domains and humanized versions, and camelized antibody variable domains.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein. In some embodiments, the CDR sequences provided herein are based on IMGT definition. For example, the CDR sequences may be determined by the VBASE2 tool (www.vbase2.org/vbase2.php, see also Retter I, Althaus H H, Münch R, Müller W: VBASE2, an integrative V gene database. Nucleic Acids Res. 2005 Jan. 1; 33 (Database issue): D671-4, which is incorporated herein by reference in its entirety).

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, an individual suffers from a disease or condition (e.g., cancer). In some embodiments, the individual is in need of treatment.

As is understood in the art, an “effective amount” refers to an amount of an agent (e.g., a targeting conjugate) sufficient to produce a desired therapeutic outcome (e.g., reducing the severity or duration of, stabilizing the severity of, or eliminating one or more symptoms of cancer) or a desired diagnostic outcome. For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the agent may extend survival (including overall survival and progression free survival); result in an objective response (including a complete response or a partial response); relieve to some extent one or more signs or symptoms of the disease or condition; and/or improve the quality of life of the subject.

“Percent (%) amino acid sequence identity” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, 0 amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004, each of which are incorporated herein by reference in their entirety for all purposes).

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, e.g., retained/improved antigen binding, decreased immunogenicity, or improved ADCC or CDC.

TABLE 1
Exemplary Amino Acid Substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “fusion” refers to genetically joining two polypeptide fragments to provide a single continuous polypeptide (“fusion polypeptide”). The two polypeptide fragments may be directly joined to each other, or joined via another polypeptide disposed therebetween. Routine recombinant DNA techniques or chemical gene synthesis can be used to provide nucleic acids that genetically encode a fusion polypeptide.

The term “epitope” as used herein refers to the specific group of atoms or amino acids on an antigen to which an antibody binds. Two antibodies or antigen binding fragments may bind the same epitope within an antigen if they exhibit competitive binding for the antigen.

As use herein, the terms “specifically binds,” “specifically recognizing,” and “is specific for” refer to measurable and reproducible interactions, such as binding between a target and a targeting moiety (e.g., a targeting peptide or an antibody or antigen-binding fragment thereof). In certain embodiments, specific binding is determinative of the presence of the target in the presence of a heterogeneous population of molecules, including biological molecules (e.g., cell surface receptors). For example, a targeting moiety that specifically recognizes a target (which can be an epitope) is a targeting moiety (e.g., antibody) that binds this target with greater affinity, avidity, more readily, and/or with greater duration than its bindings to other molecules. In some embodiments, the extent of binding of a targeting moiety to an unrelated molecule is less than about 10% of the binding of the targeting moiety to the target as measured, e.g., by a radioimmunoassay (RIA). In some embodiments, a targeting moiety that specifically binds a target has a dissociation constant (KD) of ≤10⁻⁵ M, ≤10⁻⁶ M, ≤10⁻⁷ M, ≤10⁻¹ M, ≤10⁻¹ M, ≤10⁻¹⁰ M, ≤10⁻¹¹ M, or ≤10⁻¹² M. In some embodiments, a targeting moiety specifically binds an epitope on a protein that is conserved among the protein from different species. In some embodiments, specific binding can include, but does not require exclusive binding. Binding specificity of the targeting moiety can be determined experimentally by methods known in the art. Such methods comprise, but are not limited to Western blots, ELISA, RIA, ECL, IRMA, EIA, BIACORE™ and peptide scans.

As used herein, “link,” “conjugate,” “ligate”, and “fuse” are used interchangeably to refer to connection of two chemical moieties either covalently or non-covalently. The connection may be direct or indirect, e.g., through a linker.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., cancer), or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Targeting Conjugates

One aspect of the present application provides a targeting conjugate comprising a targeting moiety and an effector molecule, wherein the effector molecule is conjugated to the targeting moiety via a conjugation site, wherein the effector molecule can be released from the targeting conjugate via cleavage. The cleavage can occur at a diseased site, for example, in a tumor microenvironment. In some embodiments, the cleavage occurs outside of a cell, such as outside of the tumor cells in a tumor microenvironment.

In some embodiments, there is provided a targeting conjugate comprising the structure of Formula I:

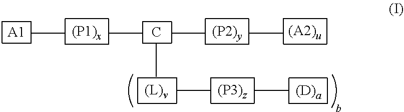

wherein: A1 is a first targeting moiety; A2 is a second targeting moiety; P1 is a first cleavage site; P2 is a second cleavage site; P3 is a third cleavage site; C is a conjugation site; L is a linker; D is an effector molecule; x=0 or 1; y=0 or 1; z=0 or 1; u=0 or 1, v=0 or 1; a=1-20; and b=1-20.

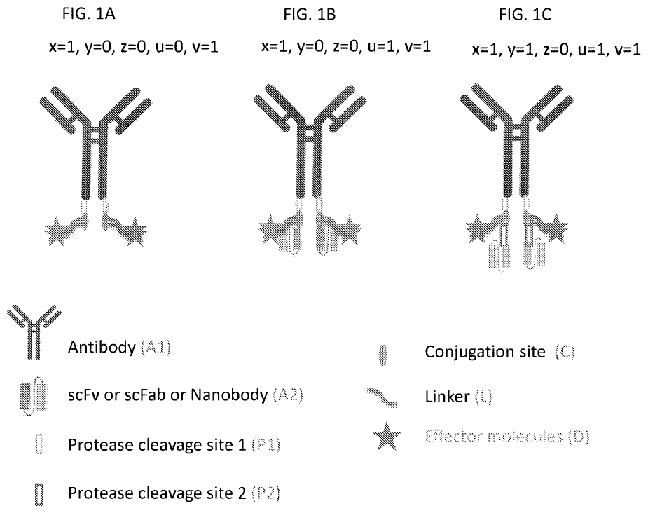

Exemplary targeting conjugates are shown in FIGS. 1A-10C, and described in section E, “Exemplary targeting conjugates” below.

The targeting moiety (A1) of the present application is conjugated to one or more effector molecules (D), which may be therapeutic agents, oligonucleotides, detectable labels and combinations thereof. In some embodiments, the effector molecule is conjugated to the targeting moiety at a conjugation site (C) via a linker (L). In some embodiments, the effector molecule is conjugated to the targeting moiety at a conjugation site (C) directly without a linker (L). In some embodiments, a cleavage site (P1, e.g., a protease cleavage site) is disposed between the conjugation site (C) and the effector molecule (D). In some embodiments, there is no cleavage site (P1, e.g., a protease cleavage site) disposed between the conjugation site (C) and the effector molecule (D). In some embodiments, the targeting conjugate comprises a second targeting moiety (A2). In some embodiments, the second targeting moiety (A2) is fused to the conjugation site (C) via a cleavage site (P2, e.g., a protease cleavage site). In some embodiments, there is no cleavage site (P2, e.g., a protease cleavage site) disposed between the second targeting moiety (A2) and the conjugation site (C). In some embodiments, the effector molecule (D) is fused to the linker via a cleavage site (P3, e.g., a protease cleavage site). In some embodiments, there is no cleavage site (P3, e.g., a protease cleavage site) disposed between the linker (L) and the effector molecule (D).

In some embodiments, there is provided a targeting conjugate comprising the structure of Formula I:

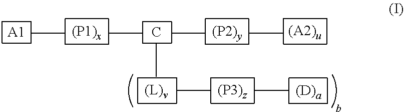

wherein: A1 is a first antibody or antigen binding fragment thereof, A2 is a second antibody or antigen binding fragment thereof, P1 is a first cleavage site (e.g., a protease cleavage site such as a uPA cleavage site); P2 is a second cleavage site (e.g., a protease cleavage site such as a uPA cleavage site); P3 is a third cleavage site (e.g., a protease cleavage site such as a uPA cleavage site); C is a conjugation site (e.g., a transglutaminase conjugation site); L is a linker; D is an effector molecule (e.g., a therapeutic agent, an oligonucleotide or a detectable label); x=0 or 1; y=0 or 1; z=0 or 1; u=0 or 1; v=0 or 1; a=1-20; and b=1-20. In some embodiments, A1 is an antibody or antigen binding fragment (e.g., a full-length antibody) that specifically binds to an immune checkpoint molecule, such as PD-L1. In some embodiments, A2 is an antibody or antigen binding fragment (e.g., a full-length antibody) that specifically binds to a tumor antigen, such as TROP-2.

In some embodiments, there is provided a targeting conjugate comprising: (a) a first polypeptide chain comprising: a first heavy chain of an anti-PD-L1 antibody, a first transglutaminase conjugation site and a first antigen-binding fragment (e.g., scFv, scFab or nanobody) of an anti-TROP2 antibody; (b) a second polypeptide chain comprising: a second heavy chain of the anti-PD-L1 antibody, a second transglutaminase conjugation site and a second antigen-binding fragment (e.g., scFv, scFab or nanobody) of an anti-TROP2 antibody; (c) a third polypeptide chain comprising a first light chain of the anti-PD-L1 antibody; (d) a fourth polypeptide chain comprising a second light chain of the anti-PD-L1 antibody; (e) a first effector molecule conjugated to the first transglutaminase conjugation site via a first linker; and (f) a second effector molecule conjugated to the second transglutaminase conjugation site via a second linker. In some embodiments, the first polypeptide chain comprises from the N-terminus to the C-terminus: the first heavy chain of the anti-PD-L1 antibody, a first protease cleavage site (e.g., a uPA cleavage site), the first transglutaminase conjugation site, a second protease cleavage site (e.g., a uPA cleavage site), and the first antigen-binding fragment (e.g., scFv, scFab or nanobody) of the anti-TROP2 antibody; and the second polypeptide chain comprises from the N-terminus to the C-terminus: the second heavy chain of the anti-PD-L1 antibody, a third protease cleavage site (e.g., a uPA cleavage site), the second transglutaminase conjugation site, a fourth protease cleavage site (e.g., a uPA cleavage site), and the second antigen-binding fragment (e.g., scFv, scFab or nanobody) of the anti-TROP2 antibody. In some embodiments, the first polypeptide chain and the second polypeptide chain each comprise from the N-terminus to the C-terminus: a heavy chain of an anti-PD-L1 antibody, a protease cleavage site (e.g., a uPA cleavage site), a transglutaminase conjugation site, and an antigen-binding fragment (e.g., scFv, scFab or nanobody) of an anti-TROP2 antibody. In some embodiments, the first polypeptide chain and the second polypeptide chain each comprise from the N-terminus to the C-terminus: a heavy chain of an anti-PD-L1 antibody, a transglutaminase conjugation site, a protease cleavage site (e.g., a uPA cleavage site), and an antigen-binding fragment (e.g., scFv, scFab or nanobody) of an anti-TROP2 antibody. In some embodiments, the first polypeptide chain and the second polypeptide chain each comprise from the N-terminus to the C-terminus: a heavy chain of an anti-PD-L1 antibody, a transglutaminase conjugation site, and an antigen-binding fragment (e.g., scFv, scFab or nanobody) of an anti-TROP2 antibody. In some embodiments, the first effector molecule and the second effector molecule are identical. In some embodiments, the first effector molecule and the second effector molecule are different. In some embodiments, the first effector molecule (including the first linker), and/or the second effector molecule (including the second linker) is a therapeutic agent selected from SN38, MMAE and derivatives thereof, e.g., compounds of Formulae (1)-(7). In some embodiments, the first effector molecule and/or the second effector molecule is an oligonucleotide, such as a CpG oligonucleotide. In some embodiments, the CpG oligonucleotide comprises a nucleic acid sequence selected from the group consisting of SEQ ID NOs: 66-67. In some embodiments, the first effector molecule and/or the second effector molecule is a detectable label. In some embodiments, two or more effector molecules are conjugated to the first transglutaminase conjugation site and/or the second transglutaminase conjugation site. In some embodiments, the first transglutaminase conjugation site and/or the second transglutaminase conjugation site comprises an amino acid sequence selected from the group consisting SEQ ID NOs: 1-12. In some embodiments, the first protease cleavage site and/or the second protease cleavage site comprises an amino acid sequence selected from the group consisting of SEQ ID NOs: 50-55. In some embodiments, the first polypeptide and/or the second polypeptide comprises an amino acid sequence having at least about 80% (e.g., at least about any one of 85%, 90%, 91%, 92%, 93%, 94%, 95%, 96%, 97%, or 99%, or 100%) sequence identity to an amino acid sequence selected from the group consisting of SEQ ID NOs: 114, 116, 118, 120, 122, 124, 126, and 128. In some embodiments, the third polypeptide and/or the fourth polypeptide comprises an amino acid sequence having at least about 80% (e.g., at least about any one of 85%, 90%, 91%, 92%, 93%, 94%, 95%, 96%, 97%, or 99%, or 100%) sequence identity to an amino acid sequence selected from the group consisting of SEQ ID NOs: 115, 117, 119, 121, 123, 125, 127, and 129.

In some embodiments, there is provided a targeting conjugate comprising: (a) a first polypeptide chain comprising from the N-terminus to the C-terminus: a first heavy chain of an anti-PD-L1 antibody and a first transglutaminase conjugation site; (b) a second polypeptide chain comprising from the N-terminus to the C-terminus: a second heavy chain of the anti-PD-L1 antibody and a second transglutaminase conjugation site; (c) a third polypeptide chain comprising a first light chain of the anti-PD-L1 antibody; (d) a fourth polypeptide chain comprising a second light chain of the anti-PD-L1 antibody; (e) a first effector molecule conjugated to the first transglutaminase conjugation site via a first linker; and (f) a second effector molecule conjugated to the second transglutaminase conjugation site via a second linker. In some embodiments, the first polypeptide chain comprises from the N-terminus to the C-terminus: the first heavy chain of the anti-PD-L1 antibody, a first protease cleavage site (e.g., a uPA cleavage site) and the first transglutaminase conjugation site; and the second polypeptide chain comprises from the N-terminus to the C-terminus: the second heavy chain of the anti-PD-L1 antibody, a second protease cleavage site (e.g., a uPA cleavage site) and the second transglutaminase conjugation site. In some embodiments, the first polypeptide chain and the second polypeptide chain each comprise from the N-terminus to the C-terminus: a first heavy chain of an anti-PD-L1 antibody and a first transglutaminase conjugation site. In some embodiments, the first effector molecule and the second effector molecule are identical. In some embodiments, the first effector molecule and the second effector molecule are different. In some embodiments, the first effector molecule (including the first linker), and/or the second effector molecule (including the second linker) is a therapeutic agent selected from SN38, MMAE and derivatives thereof, e.g., compounds of Formulae (1)-(7). In some embodiments, the first effector molecule and/or the second effector molecule is an oligonucleotide, such as a CpG oligonucleotide. In some embodiments, the CpG oligonucleotide comprises a nucleic acid sequence selected from the group consisting of SEQ ID NOs: 66-67. In some embodiments, the first effector molecule and/or the second effector molecule is a detectable label. In some embodiments, two or more effector molecules are conjugated to the first transglutaminase conjugation site and/or the second transglutaminase conjugation site. In some embodiments, the first transglutaminase conjugation site and/or the second transglutaminase conjugation site comprises an amino acid sequence selected from the group consisting SEQ ID NOs: 1-12. In some embodiments, the first protease cleavage site and/or the second protease cleavage site comprises an amino acid sequence selected from the group consisting of SEQ ID NOs: 50-55. In some embodiments, the anti-PD-L1 antibody is duravalumab, atezolizimab or a derivative thereof. In some embodiments, the first polypeptide and/or the second polypeptide comprises an amino acid sequence having at least about 80% (e.g., at least about any one of 85%, 90%, 91%, 92%, 93%, 94%, 95%, 96%, 97%, or 99%, or 100%) sequence identity to an amino acid sequence selected from the group consisting of SEQ ID NOs: 100, 102, 104, 106, 108, 110, and 112. In some embodiments, the third polypeptide and/or the fourth polypeptide comprises an amino acid sequence having at least about 80% (e.g., at least about any one of 85%, 90%, 91%, 92%, 93%, 94%, 95%, 96%, 97%, or 99%, or 100%) sequence identity to an amino acid sequence selected from the group consisting of SEQ ID NOs: 101, 103, 105, 107, 109, 111, and 113.

In some embodiments, the targeting conjugate may comprise any suitable number of the effector molecules (D), the conjugation sites (C), the protease cleavage sites (P1, P2, and P3), and the linkers (L). In some embodiments, the number of the conjugation site C can be any one of 1, 2, 3, 4, 5, 6, 7, 8, 9, 10, 11, 12, 13, 14, 15, 16, 17, 18, 19, or 20. In some embodiments, the number of the effector molecule (D) can be any one of 1, 2, 3, 4, 5, 6, 7, 8, 9, 10, 11, 12, 13, 14, 15, 16, 17, 18, 19, or 20. In some embodiments, the number of the first protease cleavage site P1 can be any one of 0, 1, 2, 3, 4, 5, 6, 7, 8, 9, 10, 11, 12, 13, 14, 15, 16, 17, 18, 19, or 20. In some embodiments, the number of the second protease cleavage site P2 can be any one of 0, 1, 2, 3, 4, 5, 6, 7, 8, 9, 10, 11, 12, 13, 14, 15, 16, 17, 18, 19, or 20. In some embodiments, the number of the third protease cleavage site P3 can be any one of 0, 1, 2, 3, 4, 5, 6, 7, 8, 9, 10, 11, 12, 13, 14, 15, 16, 17, 18, 19, or 20. In some embodiments, the number of the linker L can be any one of 0, 1, 2, 3, 4, 5, 6, 7, 8, 9, 10, 11, 12, 13, 14, 15, 16, 17, 18, 19, or 20.

The target conjugate may comprise any one of the targeting moieties, effector molecules, conjugation sites, cleavage sites (e.g., protease cleavage sites), and linkers described in sections A-D described below.

In some embodiments, the targeting conjugate comprises a single effector molecule. In some embodiments, the targeting conjugate comprises a plurality of effector molecules. In some embodiments, the targeting conjugate comprises a single molecule of the effector molecule. In some embodiments, the targeting conjugate comprises two or more of the same effector molecule. In some embodiments, the targeting conjugate comprises two or more different effector molecules. In some embodiments, the targeting conjugate comprises a single copy of each effector molecule. In some embodiments, the targeting conjugate comprises two or more copies of each effector molecule.

In some embodiments, the targeting conjugate has a high drug loading. The term “drug loading” refers to the ratio between the number of effector molecules to the targeting moiety (e.g., an antibody or antigen-binding fragment thereof) in the targeting conjugate. For example, an antibody conjugated to a total of 8 effector molecules has a drug loading of 8. Each molecule of a targeting conjugate has an integer value of drug loading. However, in a composition, different molecules of a targeting conjugate may have different values of drug loading. Thus, a composition may have an average drug loading of an integer value or a non-integer value.

In some embodiments, the targeting conjugate has a ratio of the effector molecule to the targeting moiety (e.g., the first targeting moiety and/or the second targeting moiety) of at least about any one of 1:1, 2:1, 3:1, 4:1, 5:1, 6:1, 7:1, 8:1, 9:1, 10:1, 11:1, 12:1, 13:1, 14:1, 15:1, 16:1, 17:1, 17:1, 18:1, 19:1, or 20:1. In some embodiments, the targeting conjugate has a ratio of the effector molecule to the targeting moiety (e.g., the first targeting moiety and/or the second targeting moiety) of no more than about any one of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments the targeting conjugate has a ratio of the effector molecule to the targeting moiety (e.g., the first targeting moiety and/or the second targeting moiety) of about any one of 1:1-2:1, 2:1-4:1, 4:1-8:1, 1:1-10:1, 1:1-16:1, 4:1-20:1, 10:1-20:1, 1:1-20:1, or 2:1-20:1.

In some embodiments, the drug loading of the targeting conjugate is at least about any one of 2:1, 3:1, 4:1, 5:1, 6:1, 7:1, 8:1, 9:1, 10:1, 11:1, 12:1, 13:1, 14:1, 15:1, 16:1, 17:1, 17:1, 18:1, 19:1, 20:1, 25:1, 30:1, 35:1, 40:1, 45:1, 50:1, 60:1, 70:1, 80:1, 90:1, 100:1 or more. In some embodiments, the drug loading of the targeting conjugate is no more than about any one of 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, or 2:1. In some embodiments, the drug loading of the targeting conjugate is about any one of 2:1-4:1, 2:1-8:1, 2:1-10:1, 2:1-16:1, 4:1-20:1, 10:1-20:1, 20:1-40:1, 40:1-100:1, 2:1-20:1, 2:1-40:1, or 10:1-40:1.

In some embodiments, the targeting conjugate has a ratio of the oligonucleotide to the targeting moiety (e.g., the first targeting moiety and/or the second targeting moiety) of at least about any one of 1:1, 2:1, 3:1, 4:1, 5:1, 6:1, 7:1, 8:1, 9:1, 10:1, 11:1, 12:1, 13:1, 14:1, 15:1, 16:1, 17:1, 17:1, 18:1, 19:1, or 20:1. In some embodiments, the targeting conjugate has a ratio of the oligonucleotide to the targeting moiety (e.g., the first targeting moiety and/or the second targeting moiety) of no more than about any one of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments the targeting conjugate has a ratio of the oligonucleotide to the targeting moiety (e.g., the first targeting moiety and/or the second targeting moiety) of about any one of 1:1-2:1, 2:1-4:1, 4:1-8:1, 1:1-10:1, 1:1-16:1, 4:1-20:1, 10:1-20:1, 1:1-20:1, or 2:1-20:1.

Also provided are methods of preparing the targeting conjugate, comprising conjugating an effector molecule to a targeting moiety.

### A. Targeting Moiety

The targeting conjugates of the present applications comprise one or more targeting moieties. In some embodiments, the targeting conjugate comprises a first targeting moiety that specifically binds to a first target molecule, a second targeting moiety that specifically binds to a second target molecule, and a conjugation site, wherein the first targeting moiety is fused to the second targeting moiety via the conjugation site. In some embodiments, the first targeting moiety and the second targeting moiety are identical. In some embodiments, the first targeting moiety and the second targeting moiety are different. In some embodiments, the first targeting moiety and the second targeting moiety specifically bind to the same targeting molecule. In some embodiments, the first targeting moiety and the second targeting moiety specifically bind to different targeting molecule. In some embodiments, a cleavage site (e.g., a protease cleavage site) is disposed between the first targeting moiety and the conjugation site. In some embodiments, a cleavage site (e.g., a protease cleavage site) is disposed between the second targeting moiety and the conjugation site.

### Antibody or Antigen-Binding Fragment Thereof

In some embodiments, the targeting moiety comprises an antibody, or an antigen binding fragment thereof. In some embodiments, the target moiety is a fusion protein comprising an antibody or antigen binding fragment thereof. In some embodiments, the targeting moiety is a monoclonal antibody. In some embodiments, the targeting moiety is a full-length antibody. In some embodiments, the targeting moiety is an antigen-binding fragment. Exemplary antigen-binding fragments include, but are not limited to, a single-chain Fv (scFv), a Fab, a Fab′, a F(ab′)₂, an Fv, a disulfide stabilized Fv fragment (dsFv), a (dsFv)₂, a single-domain antibody (e.g., VHH), an Fv-Fc fusion, an scFv-Fc fusion, an scFv-Fv fusion, a diabody, a tribody, and a tetrabody.

In some embodiments, the targeting moiety comprises an scFv. In some embodiments, the scFv comprises from the N-terminus to the C-terminus: VL-VH, wherein the dash is a bond or a peptide linker. In some embodiments, the scFv comprises from the N-terminus to the C-terminus: VH-VL, wherein the dash is a bond or a peptide linker. In some embodiments, the targeting moiety is a fusion protein comprising an scFv. In some embodiments, the fusion protein is an scFv-Fc fusion protein. In some embodiments, the fusion protein is an scFv-Fv fusion protein. In some embodiments, the fusion protein is an scFv-full length antibody fusion protein. In some embodiments, the N terminus of the scFv is covalently fused to the C-terminus of a heavy chain or a light chain of a full length antibody.

In some embodiments, the targeting moiety is a Fab or Fab′. In some embodiments, the targeting moiety is a Fab-containing polypeptide, which may comprise part or all of a wild-type hinge sequence (generally at the carboxyl terminus of the Fab portion of the polypeptide). A Fab-containing polypeptide may be obtained or derived from any suitable immunoglobulin. In some embodiments, a Fab-containing polypeptide may be a Fab-fusion protein that combines a Fab fragment with a fusion partner, such as the target-binding region of a receptor, an adhesion molecule, a ligand, an enzyme, a cytokine, a chemokine, or some other protein or protein domain. In some embodiments, the targeting peptide is a domain or a portion of a target protein.

In some embodiments, the targeting moiety comprises a nanobody, also known as a single domain antibody (sdAb). Exemplary sdAbs include, but are not limited to, heavy chain variable domains from heavy-chain only antibodies (e.g., VHH or VNAR), binding molecules naturally devoid of light chains, single domains (such as VH or VL) derived from conventional 4-chain antibodies, humanized heavy-chain only antibodies, human sdAbs produced by transgenic mice or rats expressing human heavy chain segments, and engineered domains and single domain scaffolds other than those derived from antibodies. In some embodiments, the targeting moiety comprises a VHH.

In some embodiments, the targeting moiety comprises a multispecific antibody. In some embodiments, the targeting moiety is a multispecific antibody. In some embodiments, the multispecific antibody is a bispecific antibody. Exemplary bispecific antibodies include full-length and Fab 2 constructs, as well as diabodies. In some embodiments, the diabody is a noncovalent dimer of single-chain Fv (scFv) fragments connected by a peptide linker. In some embodiments, another form of diabody is single-chain (Fv), in which two scFv fragments are covalently linked to each other. In some embodiments, the multispecific antibody is a trispecific antibody. Exemplary trispecific antibodies include Fab3 and triabodies, with the latter being the three-scFv-version of diabodies. In some embodiments, scFv-Fc are made of two linked single chain variable fragments fused to an intact Fc region. In some embodiments, minibodies are similar to scFv-Fc, but only contain a CH1 domain rather than a full Fc region. Other multivalent constructs include IgNAR and hcIgG.

In some embodiments, the targeting moiety is a chimeric, human, partially humanized, fully humanized, or semi-synthetic antibody. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains. In some embodiments, the targeting moiety is an antagonist antibody or antigen-binding fragment thereof. In some embodiments, the targeting moiety is an agonist antibody or antigen-binding fragment thereof.

In some embodiments, the antibody or antigen-binding fragment thereof comprises one or more antibody constant regions, such as human antibody constant regions. In some embodiments, the heavy chain constant region is of an isotype selected from IgA, IgG, IgD, IgE, and IgM. In some embodiments, the human light chain constant region is of an isotype selected from κ and λ. In some embodiments, the antibody or antigen-binding fragment thereof comprises an IgG constant region, such as a human IgG1, IgG2, IgG3, or IgG4 constant region. In some embodiments, when effector function is desirable, an antibody comprising a human IgG1 heavy chain constant region or a human IgG3 heavy chain constant region may be selected. In some embodiments, when effector function is not desirable, an antibody comprising a human IgG4 or IgG2 heavy chain constant region may be selected. In some embodiments, the antibody comprises a human IgG4 heavy chain constant region.

In some embodiments, the antibody or antigen binding fragment thereof comprises a stabilizing domain. In some embodiments, the stabilizing domain comprises an Fc domain. The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, the Fc domain is selected from the group consisting of Fc fragments of IgG, IgA, IgD, IgE, IgM, and combinations and hybrids thereof. In some embodiments, the Fc domain is derived from a human IgG. In some embodiments, the Fc domain comprises the Fc domain of human IgG1, IgG2, IgG3, IgG4, or a combination or hybrid IgG. In some embodiments, the Fc domain has a reduced effector function as compared to corresponding wild type Fc domain (such as at least about 30%, 40%, 50%, 60%, 70%, 80%, 85%, 90%, or 95% reduced effector function as measured by the level of antibody-dependent cellular cytotoxicity (ADCC)). In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. In some embodiments, the targeting moiety comprises a variant Fc region has at least one amino acid substitution compared to the Fc region of a wild type IgG or a wild-type antibody.

An antibody may consist of two identical light protein chains (light chains) and two identical heavy protein chains (heavy chains), all held together covalently by interchain disulfide linkages. The N-terminal regions of the light and heavy chains together can form the antigen recognition site of the antibody. Structurally, various functions of an antibody can be confined to discrete protein domains or regions. In some embodiments, the antibody comprises an antigen binding portion comprises a heavy chain comprising the VH and a light chain comprising the VL. Antigen-binding antigen binding fragments, including single-chain antibodies, may comprise the variable region(s) alone or in combination with the entirety or a portion of the following: hinge region, CH1, CH2, and CH3 domains.

In some embodiments, the antibody is altered to increase or decrease the extent to which the antibody is glycosylated. Addition or deletion of glycosylation sites to an antibody may be conveniently accomplished by altering the amino acid sequence such that one or more glycosylation sites is created or removed. In some embodiments, the antibody is altered to increase or decrease the reactivity of the reactive functional groups on the antibody. In some embodiments, the antibody is altered to increase or decrease the number of reactive functional groups. In some embodiments, the antibody is altered to increase or decrease the extent to which reactive functional groups are exposed. The reactive functional groups can be at the N terminus, the C terminus, or in the sidechains of the amino acids of the antibody. The reactive functional group can be naturally occurring in the antibody or incorporated. The reactive functional group can be an amine or a derivative thereof, a carboxyl group or a derivative thereof, a nitro, or other functional groups. In some embodiments, the reactive functional group is an amine. In some embodiments, the amine is in the Fc region of the antibody.

Antibodies that specifically bind to a target molecule can be obtained using methods known in the art, such as by immunizing a non-human mammal and obtaining hybridomas therefrom, or by cloning a library of antibodies using molecular biology techniques known in the art and subsequence selection or by using phage display. Nucleic acid constructs encoding any one of the antibodies or antigen-binding fragments described herein, vectors, and host cells for preparation are also provided.

### Targeting Peptide

In some embodiments, the targeting moiety comprises a targeting peptide. The term “targeting peptide” refers to a non-antibody based polypeptide that specifically binds to a target molecule, e.g., a cell surface molecule at a target site. In some embodiments, the targeting moiety is a fusion protein comprising an antibody or antigen-binding fragment thereof fused to a targeting peptide.

In some embodiments, the targeting polypeptide comprises a non-antibody scaffold. Non-antibody scaffolds are engineered protein scaffolds that yield specificities towards different kinds of targets. Compared with antibodies, engineered protein scaffolds offer a much smaller size and simpler architecture that facilitates recombinant gene expression, the construction of bifunctional fusion proteins, and tissue penetration. See, for example, Skerra, Curr. Opin. Biotech. 2007, 18:298-304. Over 50 different non-antibody scaffolds have been reported. See, for example, Vazquez-Lombardi, Rodrigo, et al. Drug discovery today 20.10 (2015): 1271-1283. Exemplary non-antibody scaffolds include, but are not limited to, a lipocalin, an anticalin (artificial antibody mimetic proteins that are derived from human lipocalins), ‘T-body’, a peptide (e.g., a BICYCLE™ peptide), an affibody (antibody mimetics composed of alpha helices, e.g. an three-helix bundle), a peptibody (peptide-Fc fusion), a DARPin (designed ankyrin repeat proteins, engineered antibody mimetic proteins consisting repeat motifs), an affimer, an avimer, a knottin (a protein structural motif containing 3 disulfide bridges), a monobody, an affinity clamp, an ectodomain, a receptor ectodomain, a receptor, a cytokine, a ligand, an immunocytokine, and a centryin. See, for example, WO2019084060, which is incorporated herein by reference.

In some embodiments, the targeting moiety comprises an anticalin. Anticalins are among the more actively developed non-antibody scaffolds, with a high number of lead compounds under preclinical development directed against CTLA-4, hepcidin, hepatocyte growth factor receptor (HGFR; MET), IL-4Ra and IL-23/IL-17. PRS-050 (ANGIOCAL®; Pieris), is an antiangiogenic Anticalin targeting VEGF-A currently undergoing Phase I clinical investigation. A VEGF-A targeting DARPin, MP0112 (Molecular Partners/Allergan) targeting retinal angiogenic disorders, is also currently being assessed in clinical studies. FDA-approved single non-antibody scaffolds include the Kunitz domain KALBITOR® (ecallantide; Dyax), a plasma kallikrein inhibitor used for the treatment of hereditary angioedema; BERINERT® (CSL-Behring), and CINRYZE® (ViroPharma/Shire), as well as the bradykinin receptor antagonist FIRAZYR® (Shire). See, for example, Vazquez-Lombardi, Rodrigo, et al. Drug discovery today 20.10 (2015): 1271-1283.

In some embodiments, the targeting peptide comprises a polypeptide derived from a receptor or a ligand of the target molecule. In some embodiments, the targeting polypeptide is an inhibitory polypeptide that blocks the binding of the target molecule and its ligand or receptor completely or partially such as by at least about any one of 5%, 10%, 15%, 20%, 25%, 30%, 35%, 40%, 45%, 50%, 60%, 70%, 80%, 90%, or 95%. In some embodiments, the targeting peptide is at least about any one of 2, 5, 10, 15, 20, 25, 30, 35, 40, 45, 50, 100, 150, 200, 250, 300, 350, 400, 450, 500 or more amino acids in length.

In some embodiments, the targeting peptide comprises a stabilizing domain. The stabilizing domain can be any suitable domain that stabilizes the targeting peptide. In some embodiments, the stabilizing domain extends the half-life of the targeting peptide in vivo. In some embodiments, the stabilizing domain is an Fc domain, such as any one of the Fc domains described in the “Antibodies or antigen-binding fragment thereof” section. In some embodiments, the stabilizing domain is an albumin domain. In some embodiments, the targeting peptide and the stabilization domain are fused to each other via a linker, such as a peptide linker.

A peptide linker may have a naturally occurring sequence, or a non-naturally occurring sequence. For example, a sequence derived from the hinge region of heavy chain only antibodies may be used as the linker. The peptide linker can be of any suitable length. In some embodiments, the peptide linker tends not to adopt a rigid three-dimensional structure, but rather provide flexibility to a polypeptide. In some embodiments, the peptide linker is a flexible linker. Exemplary flexible linkers include glycine polymers, glycine-serine polymers, glycine-alanine polymers, alanine-serine polymers, and other flexible linkers known in the art. In some embodiments, the peptide linker comprises substrate sequences for enzymatic reactions. In some embodiments, the peptide linker comprises substrate sequences for enzymes that ligates the targeting peptide and a stabilizing domain.

The targeting peptide may be obtained using known methods in the art, such as by screening a library of polypeptides. The polypeptides can be prepared using chemical synthesis or produced using recombinant DNA techniques. Nucleic acid constructs encoding any one of the targeting peptides described herein, vectors, and host cells for preparation are also provided.

### Target Molecule and Exemplary Targeting Moieties

The targeting moiety specifically binds to one or more target molecules. In some embodiments, the targeting moiety is monospecific, i.e., specifically binds to a single target molecule. In some embodiments, the targeting moiety is multispecific, i.e., specifically binds to two or more different target sites in a single target molecule, or two or more different target molecules. In some embodiments, the targeting moiety has a single target binding site. In some embodiments, the targeting moiety has two or more target binding sites.

In some embodiments, the targeting moiety specifically binds to a cell surface molecule. In some embodiments, the cell surface molecule is a cell surface molecule at a target site. In some embodiments, the target site is site of a disease. In some embodiments, the disease is a tumor. In some embodiments, the disease is an inflammatory disease. In some embodiments, the disease is a fibrotic disease. In some embodiments, the disease is an infection. In some embodiments, the disease is an autoimmune disease. In some embodiments, the disease is an immunodeficiency disease

Exemplary target molecules include but are not limited to proteins, glycans, lipids, other small molecules, or a combination thereof. In some embodiments, the cell surface molecule is a tumor-specific marker/tumor antigen. As described herein, a “tumor-specific marker” or a “tumor antigen” refers to a molecular marker that can be expressed on a neoplastic tumor cell and/or within a tumor microenvironment. A tumor antigen can be a tumor specific antigen and/or a tumor associated antigen. For example, a tumor antigen can be an antigen expressed on a cell associated with a tumor, such as a neoplastic cell, stromal cell, endothelial cell, fibroblast, or tumor-infiltrating immune cell. For example, the tumor antigen Her2/Neu can be overexpressed by certain types of breast and ovarian cancer. A tumor antigen can also be ectopically expressed by a tumor and contribute to deregulation of the cell cycle, reduced apoptosis, metastasis, and/or escape from immune surveillance. Tumor antigens are generally proteins or polypeptides derived therefrom, but can be glycans, lipids, or other small organic molecules. Additionally, a tumor antigen can arise through increases or decreases in post-translational processing exhibited by a cancer cell compared to a normal cell, for example, protein glycosylation, protein lipidation, protein phosphorylation, or protein acetylation.

In some embodiments, the tumor antigen is a cell-surface carbohydrate. In some embodiments, the cell-surface carbohydrate is a monosaccharide. In some embodiments, the cell-surface glycan is an oligosaccharide. In some embodiments, the cell-surface carbohydrate is a polysaccharide. In some embodiments, the cell-surface carbohydrate is a glycan. In some embodiments, the cell-surface carbohydrate is a glycoconjugate, such as a glycoprotein, glycolipid, or a proteoglycan. In some embodiments, the cell-surface glycan is selected from the group consisting of GD2, GD3, GM2, Le^{y}, sLe, polysialic acid, fucosyl GMl, Tn, STn, BM3, or GloboH.

In some embodiments, the tumor antigen is a cell-surface protein. In some embodiments, the cell-surface protein is selected from the group consisting of CD5, CD19, CD20, CD25, CD37, CD30, CD33, CD45, CAMPATH-1, BCMA, CS-1, PD-1, PD-L1, B7-H3, B7-DC (PD-L2), HLA-DR, carcinoembryonic antigen (CEA), TAG-72, MUC1, MUC15, MUC16, folate-binding protein, A33, G250, prostate specific membrane antigen (PSMA), CA-125, CA19-9, epidermal growth factor, HER2, IL-2 receptor, EGFRvIII (de2-7 EGFR), EGFR, fibroblast activation protein (FAP), tenascin, a metalloproteinase, endosialin, vascular endothelial growth factor, αvβ3, WTI, LMP2, HPV E6, HPV p53 nonmutant, NY-ESO-1, GLP-3, MelanA/MARTI, Ras mutant, gp100, p53 mutant, PRI, bcr-abl, tyrosinase, survivin, PSA, hTERT, STNI, TNC, a Sarcoma translocation breakpoint fusion protein, EphA2, PAP, ML-IAP, AFP, ERG, NAI 7, PAX3, ALK, androgen receptor, cyclin B1, MYCN, RhoC, TRP-2, mesothelin (MSLN), PSCA, MAGE A1, MAGE-A3, CYPIBI, PLAVI, BORIS, Tn, ETV6-AN1L, NY-BR-1, RGS5, SART3, Carbonic anhydrase IX, PAX5, OY-TESI, Sperm protein 17, LCK, MAGE C2, MAGE A4, GAGE, TRAILI, HMWMAA, AKAP-4, SSX2, XAGE 1, B7H3, Legumain, Tie 3, PAGE4, VEGFR2, MAD-CTI, PDGFR-B, MAD-CT-2, ROR2, CMET, HER3, EPCAM, CA6, NAPI2B, TROP2, Claudin-6 (CLDN6), Claudin-16 (CLDN16), CLDN18.2, RON, LY6E, FRA, DLL3, PTK7, Uroplakin-IB (UPKIB), LIVI, RORI, STRA6, TMPRSS3, TIVIPRSS4, TIV1EM238, Clorf186, Fos-related antigen 1, VEGFR1, endoglin, VTCNI (B7-H4), VISTA, and fragments thereof.

In some embodiments, the tumor antigen is selected from the group consisting of PD1, PD-L1, Trop2, CTLA-4, LAG-3, TIM-3, 4-1BB, CD40, OX40, CD47, SIRPα, HER2, HER3, EGFR, VEGF, VEGR2, CD19, CD20, CD22, CD30, CD33, CD38, CD79, integrin αvβ3, αvβ6, MUC1, PMSA, uPAR, and Angiopep-2. In some embodiments, the tumor antigen is Trop2. Trop-2, also known as epithelial glycoprotein-1, gastrointestinal antigen 733-1, membrane component surface marker-1, and tumor-associated calcium signal transducer-2, is the protein product of the TACSTD2 gene. Trop-2 is a transmembrane glycoprotein that is upregulated in all cancer types independent of baseline levels of Trop-2 expression. Trop-2 is an ideal candidate for targeted therapeutics due to it being a transmembrane protein with an extracellular domain overexpressed on a wide variety of tumors as well as its upregulated expression relative to normal cells. See, for example, Onco Targets Ther. 2019; 12: 1781-1790.

In some embodiments, the target molecule is associated with fibrotic or inflammatory disease. In some embodiments, the target molecule is selected from the group consisting of Cadherin 11, PDPN, LRRC15, Integrin a, 4f37, Integrin a2f31, MADCAM, Nephrin, Podocin, IFNARI, BDCA2, CD30, c-KIT, FAP, CD73, CD38, PDGFRf3, Integrin avf31, Integrin avf33, Integrin avf38, GARP, Endosialin, CTGF, Integrin avf36, CD40, PD-1, TIM-3, TNFR2, DEC205, DCIR, CD86, CD45RB, CD45RO, MHC Class II, CD25, LRRC15, MMP14, GPX8, and F2RL2.

In some embodiments, the target molecule is associated with an infection or an immunodeficiency disease. In some embodiments, the target molecule is a protein of an infectious agent, such as a pathogenic bacterium or a virus.

In some embodiments, the targeting moiety specifically binds to an immune checkpoint protein. Immune checkpoint proteins regulate immune activation. However, some cancers can protect themselves from attack by stimulating immune checkpoint targets. See, for example, Nature Reviews. Cancer. 12 (4): 252-64. Inhibitory checkpoint molecules are targets for cancer immunotherapy due to their potential for use in multiple types of cancers. In some embodiments, the targeting moiety comprises an immune checkpoint inhibitor. Immune checkpoint inhibitors are compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. Immune checkpoint inhibitors can inhibit an immune system checkpoint by stimulating the activity of a stimulatory checkpoint molecule, or inhibiting the activity of an inhibitory checkpoint molecule in the pathway. Immune system checkpoint molecules include, but are not limited to, cytotoxic T-lymphocyte antigen 4 (CTLA-4), programmed cell death 1 protein (PD-1), programmed cell death 1 ligand 1 (PD-L1), programmed cell death 1 ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, immune checkpoint inhibitors include antagonists of CTLA-4, PD-1, PD-L1, PD-L2, LAG3, B7-1, B7-H3, B7-H4, BTLA, VISTA, KIR, A2aR, and TIM3. For example, antibodies that bind to CTLA-4, PD-1, PD-L1, PD-L2, LAG3, B7-1, B7-H3, B7-H4, BTLA, VISTA, KIR, A2aR, or TIM3 and antagonize their function are immune checkpoint inhibitors.

In some embodiments, the targeting moiety comprises an antibody or antigen-binding fragment that specifically binds to an immune checkpoint molecule. In some embodiments, the targeting moiety comprises an anti-PD-L1 antibody or antigen binding fragment thereof. In some embodiments, the anti-PD-L1 antibody is derived from any anti-PD-L1 antibodies known in the art. Exemplary anti-PD-L1 antibodies include, but are not limited to, atezolizumab, avelumab, Durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, FS-118, biosimilars thereof, and derivatives thereof. In some embodiments, the antibodies that compete with any of these art-recognized antibodies for binding to PD-L1 also can be used. In some embodiments, the anti-PD-L1 antibody is a derivative of any one of the anti-PD-L1 antibodies described herein. In some embodiments, the anti-PD-L1 antibody is derived from an antibody selected from the group consisting of Durvalumab, atezolizumab, and avelumab.

In some embodiments, the anti-PD-L1 antibody is derived from Durvalumab. In some embodiments, the targeting moiety binds competitively to the same or substantially the same epitope as Durvalumab. In some embodiments, the anti-PD-L1 antibody comprises a VH and a VL, wherein the VH comprises a CDR H1 comprising the amino acid sequence of SEQ ID NO:86, a CDR H2 comprising the amino acid sequence of SEQ ID NO:87, and a CDR H3 comprising the amino acid sequence of SEQ ID NO:88; and wherein the VL comprises a CDR L1 comprising the amino acid sequence of SEQ ID NO:89, a CDR L2 comprising the amino acid sequence of SEQ ID NO:90, and a CDR L3 comprising the amino acid sequence of SEQ ID NO:91. In some embodiments, the anti-PD-L1 antibody comprises a heavy chain comprising the amino acid sequence of SEQ ID NO 84; and/or a light chain comprising the amino acid sequence of SEQ ID NO:85.

In some embodiments, the anti-PD-L1 antibody is derived from atezolizumab. In some embodiments, the targeting moiety binds competitively to the same or substantially the same epitope as atezolizumab. In some embodiments, the anti-PD-L1 antibody comprises a VH and a VL, wherein the VH comprises a CDR H1 comprising the amino acid sequence of SEQ ID NO:94, a CDR H2 comprising the amino acid sequence of SEQ ID NO:95, and a CDR H3 comprising the amino acid sequence of SEQ ID NO:96; and wherein the VL comprises a CDR L1 comprising the amino acid sequence of SEQ ID NO:97, a CDR L2 comprising the amino acid sequence of SEQ ID NO:98, and a CDR L3 comprising the amino acid sequence of SEQ ID NO:99. In some embodiments, the anti-PD-L1 antibody comprises a heavy chain comprising the amino acid sequence of SEQ ID NO: 92, and/or a light chain comprising the amino acid sequence of SEQ ID NO: 93.

In some embodiments, the targeting moiety comprises a variant of the targeting moieties described herein. For example, a variant of an antibody may comprise one or more modifications of the amino acid sequences of an illustrative antibody (“parent antibody”) while conserving the overall molecular structure of the parent antibody amino acid sequence. Amino acid sequences of any regions of the parent antibody chains may be modified, such as framework regions, CDR regions, or constant regions. Types of modifications include substitutions, insertions, deletions, or combinations thereof, of one or more amino acids of the parent antibody. In some embodiments, the antibody variant comprises a CDR (e.g., CDR H1, CDR H2, CDR H3, CDR L1, CDR L2, or CDR L3) having at least 80% (e.g., at least about any one of 85%, 90%, 91%, 92%, 93%, 94%, 95%, 96%, 97%, or 99%) sequence identity to the corresponding CDR of a parent antibody. In some embodiments, the antibody variant comprises a VH comprising an amino acid sequence that is at least 80% (e.g., at least about any one of 85%, 90%, 91%, 92%, 93%, 94%, 95%, 96%, 97%, or 99%) identical to the VH of a parent antibody. In some embodiments, the antibody variant comprises a VL comprising an amino acid sequence that is at least 80% (e.g., at least about any one of 85%, 90%, 91%, 92%, 93%, 94%, 95%, 96%, 97%, or 99%) identical to the VL of a parent antibody. In some particular embodiments, the antibody variant comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of the VH, VL, heavy chain or light chain of a parent antibody.

In some embodiments, the targeting moiety specifically binds to a calcium signal transducer. Intracellular calcium signal transducer is involved in cell signaling, migration, proliferation, and differentiation. It has been shown that intracellular calcium signal transducers have oncogenic potential via its capacity to upregulate various proto-oncogenes and cell cycle-related pathways. In some embodiments, the intracellular calcium signal transducer is a tumor-associated calcium signal transducer. Tumor-associated calcium signal transducers are targets for cancer immunotherapy due to their differential expression in multiple types of cancers. In some embodiments, the targeting moiety specifically binds to tumor-associated calcium signal transducer 2 (Trop-2).

Trop-2 is also known as trophoblast cell-surface antigen 2, M1S1, GA733-1, EGP-1, or TACSTD2. Trop-2 is a cell surface glycoprotein originally identified in human placental trophoblast and subsequently found to be highly expressed in most human carcinomas, but showed only restricted or limited expression in normal adult tissues. See, e.g., Varughese et al., Gynecologic Oncology, 122:171-177, 2011. Without being bound by any theory or hypothesis, Trop-2 is involved in several cell signaling pathways, of which many are associated with tumorigenesis. For example, in thyroid cancer cell invasion, Trop-2 signal transduction has been seen as a downstream effect of the ERK and JNK pathways. Stoyanova et al. demonstrated that Trop-2 signaling enhances stem cell-like properties of cancer cells, as Trop-2 regulates proliferation and self-renewal through b-catenin signaling. It has been speculated that phosphatidylinositol 4,5-bisphosphate (PIP2) may regulate Trop-2 phosphorylation and calcium signal transduction, as the cytoplasmic domain of Trop-2 contains a PIP2-binding sequence overlapping with a protein kinase C phosphorylation site. See, for example, Zaman et al, Onco Targets Ther. 2019; 12: 1781-1790. Trop-2 has been shown to play a role in tumor progression and metastasis. See, for example, Lin et al., EMBO Mol Med. 2012 June; 4(6):472-85. An elevated expression level of Trop-2 has been shown to be prognostic for cancer recurrence including the prostate cancer. See, for example, Hsu et al., PNAS, 2020; 117(4):2032-2042. Trop-2 has emerged as a promising therapeutic target due to its overexpression in multiple cancers. Sacituzumab govitecan (IMMU-132), an anti-Trop2 antibody conjugated with SN-38, a cytotoxic agent that targets DNA replication, has shown therapeutic activity in several malignances, including triple negative breast cancer, advanced non-small cell lung cancer, and metastatic platinum-resistant urothelial carcinoma.

In some embodiments, the targeting moiety comprises an anti-Trop-2 antibody or fragments thereof. In some embodiments, the targeting moiety comprises an anti-Trop-2 scFv. In some embodiments, the targeting moiety comprises an anti-Trop-2 scFab. In some embodiments, the targeting moiety comprises an anti-Trop-2 nanobody. The anti-Trop-2 scFv, scFab, or nanobody may be derived from any anti-Trop-2 antibodies known in the art. Exemplary anti-Trop-2 antibodies include, but are not limited to, hRS7, 162-46.2, MAB650, K5-70, K5-107, K5-116-2-1, T6-16, T5-86, BR110, 3E9, 6G11, 7E6, 15E2, 18B1, 77220, KM4097, KM4590, A1, A3, 162-25.3, and antibodies produced by hybridomas AR47A6.4.2, AR52A301.5, PTA-12871, PTA-12872, PD 08019, PD 08020, and PD 08021, biosimilars thereof, and derivatives thereof. In some embodiments, the antibodies that compete with any of these art-recognized antibodies for binding to Trop-2 can also be used. In some embodiments, the anti-Trop-2 scFv, scFab, or nanobody is a derivative of any one of the anti-Trop-2 antibodies described herein. In some embodiments, the anti-Trop-2 scFv, scFab, or nanobody is derived from a group consisting of hRS7, 162-46.2 and MAB650. In some embodiments, the anti-Trop-2 scFv, scFab, or nanobody is derived from hRS7.

In some embodiments, the anti-Trop-2 antibody (e.g., scFv, scFab, or nanobody) is derived from hRS7. See, for example, U.S. Pat. No. 7,238,785. In some embodiments, the targeting moiety binds competitively to the same or substantially the same epitope as hRS7. In some embodiments, the anti-Trop-2 antibody (e.g., scFv or scFab) comprises a VH and a VL, wherein the VH comprises a CDR H1 comprising the amino acid sequence of SEQ ID NO:134, a CDR H2 comprising the amino acid sequence of SEQ ID NO:135, and a CDR H3 comprising the amino acid sequence of SEQ ID NO:136; and wherein the VL comprises a CDR L1 comprising the amino acid sequence of SEQ ID NO:137, a CDR L2 comprising the amino acid sequence of SEQ ID NO:138, and a CDR L3 comprising the amino acid sequence of SEQ ID NO:139. In some embodiments, the anti-Trop-2 antibody (e.g., scFv or scFab) comprises a VH comprising the amino acid sequence of SEQ ID NO: 140, and/or a VL comprising the amino acid sequence of SEQ ID NO: 141, 142, or 143. In some embodiments, the anti-Trop-2 scFv comprises SEQ ID NO: 131, 132, or 133. In some embodiments, the anti-Trop-2 scFab comprises SEQ ID NO: 130.

In some embodiments, the targeting moiety is a multispecific antibody comprising a full-length antibody fused to an antigen-binding fragment selected from the group consisting of an scFv, an scFab and a nanobody. In some embodiments, the multispecific antibody comprises a full-length anti-PD-L1 antibody fused to an anti-Trop-2 scFv, scFab, or nanobody. In some embodiments, the C terminus of a heavy chain of the anti-PD-L1 antibody is fused to the N-terminus of an anti-Trop-2 scFv, scFab, or nanobody. In some embodiments, the C terminus of a light chain of the anti-PD-L1 antibody is fused to the N-terminus of an anti-Trop-2 scFv, scFab, or nanobody. In some embodiments, the targeting moiety comprises any one of the anti-PD-L1 antibodies described herein.

### B. Effector Molecule

The targeting conjugate comprises one or more effector molecules. Exemplary effector molecules include, but are not limited to, therapeutic agents, oligonucleotides, and detectable labels. The targeting conjugate may comprise a single type of effector molecule, or different types of effector molecules. In some embodiments, the targeting conjugate comprises one or more therapeutic agents. In some embodiments, the targeting conjugate comprises one or more oligonucleotides. In some embodiments, the targeting conjugate comprises one or more detectable labels. In some embodiments, the targeting conjugate comprises a therapeutic agent and an oligonucleotide. In some embodiments, the targeting conjugate comprises a detectable label and a therapeutic agent. In some embodiments, the targeting conjugate comprises a detectable label and an oligonucleotide.

In some embodiments, the targeting conjugate comprises a first effector molecule and a second effector molecule. In some embodiments, the ratio between the first effector molecule (e.g., therapeutic agent) and the second effector molecule (e.g., oligonucleotide) is at least about any one of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, the ratio between the first effector molecule (e.g., therapeutic agent) and the second effector molecule (e.g., oligonucleotide) is no more than about any one of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the ratio between the first effector molecule (e.g., therapeutic agent) and the second effector molecule (e.g., oligonucleotide) is about any one of 1:10 to 10:1, 1:9 to about 9:1, 1:8 to 8:1, 1:7 to 7:1, 1:6 to 6:1, 1:5 to 5:1, 1:4 to 4:1, 1:3 to 3:1, 1:2 to 2:1, 1:10 to 1:5, 1:5 to 1:1, 1:1 to 5:1, 5:1 to or 5:10.

### Therapeutic Agent

In some embodiments, the effector molecule is a therapeutic agent. Exemplary therapeutic agents include, but are not limited to, drugs, toxins, immunomodulators, hormones, hormone antagonists, enzymes, enzyme inhibitors, radionuclides, angiogenesis inhibitors, chemotherapy agent, etc. In some embodiments, the therapeutic agent is a small molecule drug. In some embodiments, the therapeutic agent is a chemotherapeutic agent.

A wide variety of chemotherapeutic agents may be used in accordance with the present embodiments. A “chemotherapeutic agent” refers a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Exemplary chemotherapeutic agents include, but are not limited to, alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammal1 and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishes such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluorometlhylomithine (DMFO); retinoids, such as retinoic acid; capecitabine; carboplatin, procarbazine, plicomycin, gemcitabien, navelbine, famesyl-protein tansferase inhibitors, transplatinum, and pharmaceutically acceptable salts, acids, or derivatives of any of the above.

In some embodiments, the therapeutic agent is a kinase inhibitor. The group of targeted kinases comprises receptor tyrosine kinases e.g. BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, ALK, cytoplasmic tyrosine kinases e.g. c-SRC, c-YES, Abl, JAK-2, serine/threonine kinases e.g. ATM, Aurora A & B, CDKs, mTOR, PKCi, PLKs, b-Raf, S6K, STK1 1/LKB1 and lipid kinases e.g. PI3K, SKI. Exemplary small molecule kinase inhibitors include, e.g., PHA-739358, Nilotinib, Dasatinib, and PD166326, NSC 74341 1, Lapatinib (GW-572016), Canertinib (CI-1033), Semaxinib (SU5416), Vatalanib (PTK787/ZK222584), Sutent (SU1 1248), Sorafenib (BAY 43-9006) and Leflunomide (SU101). For more information see e.g. Zhang et al. 2009: Targeting cancer with small molecule kinase inhibitors. Nature Reviews Cancer 9, 28-39.”

In some embodiments, the chemotherapeutic agent is an anti-DNA repair agent. In some embodiments, the DNA damage repair and response inhibitor is selected from the group comprising a topoisomerase inhibitor, a PARP inhibitor, a RAD51 inhibitor, and an inhibitor of a DNA damage response kinase selected from CHCK1, ATM, or ATR. In some embodiments, the chemotherapy agent comprises a topoisomerase inhibitor. In some embodiments, therapeutic agent comprises a topoisomerase I inhibitor, such as camptothecins and related compounds. Topoisomerase I (TOP1) enzymes are essential in higher eukaryotes, as they are required to relax DNA supercoiling generated by transcription, replication and chromatin remodeling. Topoisomerases are particularly vulnerable to topoisomerase I inhibitors during their cleavage reaction, and can be trapped by anticancer drugs as it cleaves DNA. An alkaloid isolated from the Chinese tree Camptotheca acuminate, camptothecin is a natural product of which TOP1 is the only cellular target. Without being bound by any theory or hypothesis, camptothecin and its derivatives function by inhibition of TOP1 activity, resulting in DNA double-strand breaks and cell death. Derivatives of camptothecin have been developed for clinical use. Exemplary camptothecin derivatives include, but are not limited to, topotecan hydrochloride (Hycamtin), irinotecan hydrochloride (Camptosar), SN-38 (the active form of irinotecan), 9-NC, 9-AC, DE-310, lurtotecan GI-147211 NX 211, gimatecan (ST-1481), PEG-camptothecin, BNP-1350, DB-67, BN 80915. Two camptothecin derivatives have recently been approved by the FDA: topotecan for ovarian and lung cancers and irinotecan for colorectal cancer. See, for example, Pommier, Nature Reviews Cancer 6, 789-802(2006).

In some embodiments, therapeutic agent is 7-ethyl-10-hydroxy-20(S)-camptothecin (SN-38). SN-38 is the active metabolite of irinotecan, but has 1000 times more activity than irinotecan itself. In vitro cytotoxicity assays show that the potency of SN-38 relative to irinotecan varies from 2- to 2000-fold. Due to its low solubility and ultratoxicity, SN-38 has been developed into drug conjugates for better therapeutic effects. See, for example, U.S. Pat. Nos. 7,999,083 and 8,080,250.

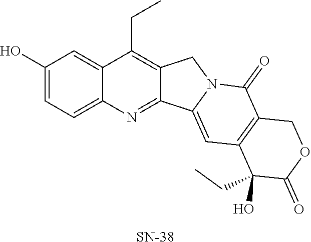 [SMILES: CCc1c2c(nc3ccc(O)cc13)-c1cc3c(c(=O)n1C2)COC(=O)[C@]3(O)CC ; "SN-38"]

In some embodiments, SN-38 is conjugated to the targeting moiety at its 10-hydroxyl position. Methods for selective regeneration of the 10-hydroxyl group in the presence of the C-20 carbonate in preparations of drug-linker precursor involving CPT analogs such as SN-38 are known in the art. See, for example, U.S. Pat. No. 10,266,605. In some embodiments, SN-38 is conjugated to the targeting conjugate at the 20-hydroxyl position. Other protecting groups for reactive hydroxyl groups in drugs such as the phenolic hydroxyl in SN-38, for example t-butyldimethylsilyl or t-butyldiphenylsilyl, may also be used, and these are deprotected by tetrabutylammonium fluoride prior to linking of the derivatized drug to an antibody-coupling moiety. The 10-hydroxyl group of CPT analogs is alternatively protected as an ester or carbonate. In some embodiments, SN-38 is conjugated to the targeting moiety via a linker.

In some embodiments, therapeutic agent comprises a mitotic inhibitor. In some embodiments, therapeutic agent comprises a tubulin disrupting agent. Microtubule/tubulin inhibitors can be classified into two major categories according to their mechanisms of action: agents promoting tubulin polymerization and stabilizing microtubule structures (e.g., paclitaxel) and agents inhibiting tubulin polymerization and destabilizing microtubule structures (such as maytansinoids, auristatins, vinblastine and vincristine). See, for example, Chen et al., Molecules 22:1281, 2017. Exemplary tubulin-disrupting agents include, but are not limited to, an auristatin, a tubulysin, a colchicine, a vnca alkaloid, a taxane, a cryptophycin, a maytansinoid, a hemiasterlin, and other tubulin disrupting agents. Auristatins are derivatives of the natural product dolastatin. Exemplary auristatins include dolastatin-10, MMAE (N-methylvaline-valine-dolaisoleuine-dolaproine-norephedrine) and MMAF (N-methylvaline-valine-dolaisoleuine-dolaproine-phenylalanine), and derivatives thereof. WO 2015/057699 describes PEGylated auristatins including MMAE. Additional dolastatin derivatives contemplated for use are disclosed in U.S. Pat. No. 9,345,785, incorporated herein by reference. Tubulysins include, but are not limited to, tubulysin D, tubulysin M, tubuphenylalanine, tubutyrosine, and derivatives thereof. Colchicines include, but are not limited to, colchicine, CA-4, and derivatives thereof. Vinca alkaloids include, but are not limited to, Vinblastine (VBL), vinorelbine (VRL), vincristine (VCR), vindesine (VDS), and derivatives thereof. Taxanes include, but are not limited to, paclitaxel, docetaxel, and derivatives thereof. Cryptophycins include but are not limited to cryptophycin-1, cryptophycin-52, and derivatives thereof. Maytansinoids include, but are not limited to, maytansine, maytansinol, maytansine analogs, DM1, DM3, DM4, ansamatocin-2, and derivatives thereof. Exemplary maytansinoid drug moieties include those having a modified aromatic ring, such as: C-19-dechloro (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (prepared by acylation using acyl chlorides), and the derivatives thereof.

Tubulin disrupting agents have been used in antibody drug conjugates for leukemia. For example, Brentuximab vedotin is an antibody-drug conjugate composed of an anti-CD30 monoclonal antibody conjugated by a protease-cleavable linker to the microtubule disrupting agent, monomethyl auristatin E. Brentuximab vedotin has been approved for the treatment of classical Hodgkin lymphoma patients after failure of autologous stem cell transplant (ASCT) or after failure of at least 2 prior multi-agent chemotherapy regimens in patients who are not ASCT candidates, and as consolidation post-ASCT for Hodgkin lymphoma patients at increased risk of relapse/progression. See, ADCETRIS® (brentuximab vedotin) US Prescribing Information and ADCETRIS® (brentuximab vedotin) EU Summary of Product Characteristics. It has also been approved for systemic anaplastic large cell lymphoma after failure of at least one prior multi-agent chemotherapy regimen.

In some embodiments, the tubulin disrupting agent comprises an auristatin. In some embodiments, therapeutic agent is monomethyl auristatin E (MMAE). MMAE is a synthetic derivative of dolastatin 10 and functions as a very potent anti-mitotic agent by inhibiting tubulin polymerization. The synthesis and structure of MMAE is described in U.S. Pat. No. 6,884,869 incorporated by reference herein in its entirety.

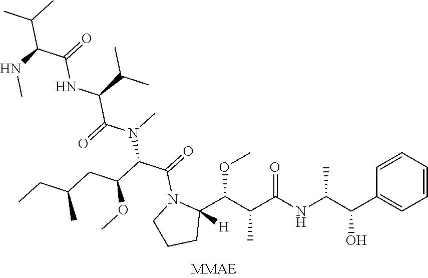 [SMILES: CC[C@H](C)C[C@H](OC)[C@@H](C(=O)N1CCC[C@H]1[C@H](OC)[C@@H](C)C(=O)N[C@H](C)[C@@H](O)c1ccccc1)N(C)C(=O)[C@@H](NC(=O)[C@@H](NC)C(C)C)C(C)C]

In some embodiments, therapeutic agent further comprises a drug linker. In some embodiments, the drug linker is conjugated to the therapeutic agent via a cleavable linker. In some embodiments, the drug linker is conjugated to the therapeutic agent via a non-cleavable linker. In some embodiments, the linker is an amine donor group linker. In some embodiments, the linker is a non-cleavable linker. Suitable non-cleavable linkers include, but are not limited to, NH₂—R—X, NH₂NH—R—X, and NH₂—O—R—X, wherein R is alkyl or polyethylene glycol group (also referred to as PEG), wherein X is the active moiety. A polyethylene glycol group or PEG group may have a formula of —(CH₂CH₂O)ₙ—, wherein n is an integer of at least 1 (e.g., 2, 3, 4, 5, 6, 7, 8, 9, 10, 11, 12, or more).

In some embodiments, the linker is a cleavable linker. Suitable cleavable linkers include, but are not limited to, Lys-Phe-X, Lys-Val-Cit-PAB-X, NH₂—(CH₂CH₂O)ₙ-Val-Cit-PAB-X, and NH₂—(CH₂CH₂O)ₙ-(Val-Cit-PAB-X)₂, wherein X is the active moiety, and n is an integer of at least 1 (e.g., 2, 3, 4, 5, 6, 7, 8, 9, 10, 11, 12, or more). PAB refers to p-aminobenzyloxycarbonyl. Cit refers to citrulline. Other exemplary amine donor group-linkers include, but are not limited to, Ac-Lys-Gly, aminocaproic acid, Ac-Lys-beta-Ala, amino-PEG₂ (Polyethylene Glycol)-C2, amino-PEG₃-C2, amino-PEG₆-C2, Ac-Lys-Val (valine)-Cit (citrulline)-PAB (p-aminobenzyloxycarbonyl), aminocaproyl-Val-Cit-PAB, putrescine, and Ac-Lys-putrescine. In some embodiments, the linker is (Gly)ₙ-(PEG)ₘ-VC-PAB-(DMAE)ₖ (Formula II), wherein n, m and k are integers, n≥1, m≥2, and k is 0 or 1. In some embodiments, the linker is (Gly)ₙ-(PEG)ₘ-VC-PAB, wherein n, and m are integers, n≥1, and m≥2. In some embodiments, the linker is (Gly)ₙ-(PEG)ₘ-Val-Ala-PAB-(DMAE)ₖ, wherein n, m and k are integers, n≥1, m≥2, and k is 0 or 1. In some embodiments, the linker is (Gly)ₙ-(PEG)ₘ-Val-Ala-PAB, wherein n and m are integers, n≥1, and m≥2. In some embodiments, the linker is (Gly)ₙ-(PEG)ₘ-P-PAB-(DMAE)ₖ (Formula III), wherein n, m and k are integers, n≥1, m≥2, and k is 0 or 1, P is a cleavage site. In some embodiments, the linker is (Gly)ₙ-(PEG)ₘ-P-PAB, wherein n and m are integers, n≥1, and m≥2.

In some embodiments, the linker is branched. In some embodiments, the linker is linear. In some embodiments, the linker has more than one (such as 2, 3, 4, 5, 6, 7, 8, 9, 10, 11, 12, 13, 14, 15, 16, 17, 18, 19, 20, or more) attachment sites for the attachment of therapeutic agents. These therapeutic agents can be the same or different from each other.

In some embodiments, the therapeutic agent is Glycine₃-amido-PEG₈-VC-PAB-DMAE-phenol linked SN38, shown as compound of Formula (1) below, wherein the linker is connected to position 10 of SN38 and has an 8-PEG spacer:

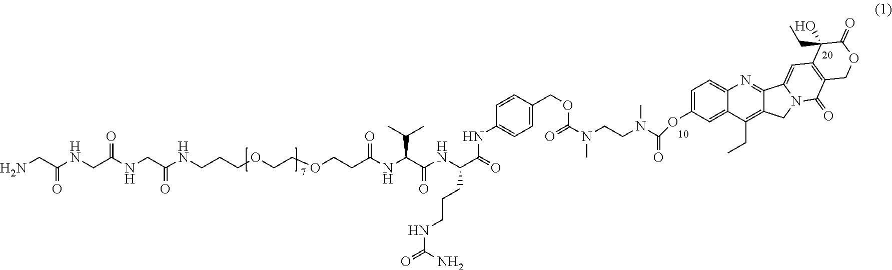

In some embodiments, the therapeutic agent is Glycine₃-amido-PEG₈-VC-PAB-DMAE-α-hydroxy lactone linked SN38, shown as compound of Formula (2) below, wherein the linker is connected to position 20 of SN38 and has an 8-PEG spacer:

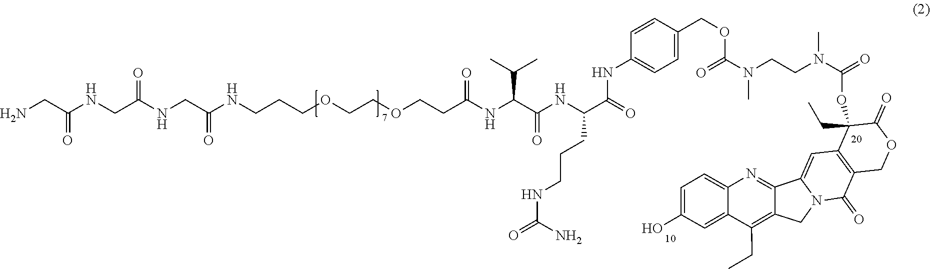

In some embodiments, the therapeutic agent is (Glycine)₃-(PEG)₄-VC-PAB-MMAE, shown as compound of Formula (3) below, wherein the linker is connected to MMAE and has a 4-PEG spacer:

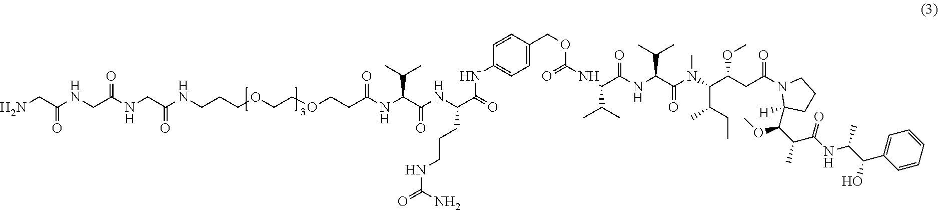

In some embodiments, the therapeutic agent is (Glycine)₃-(PEG)₈-VC-PAB-MMAE, shown as compound of Formula (4) below, wherein the linker is connected to MMAE and has an 8-PEG spacer:

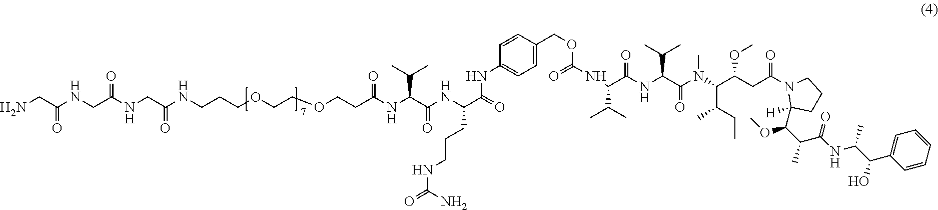

In some embodiments, the therapeutic agent is (Glycine)₃-amido-PEG₈-LGGSGRNAQVRLE-PAB-DMAE-phenol linked SN38, shown as compound of Formula (5) below:

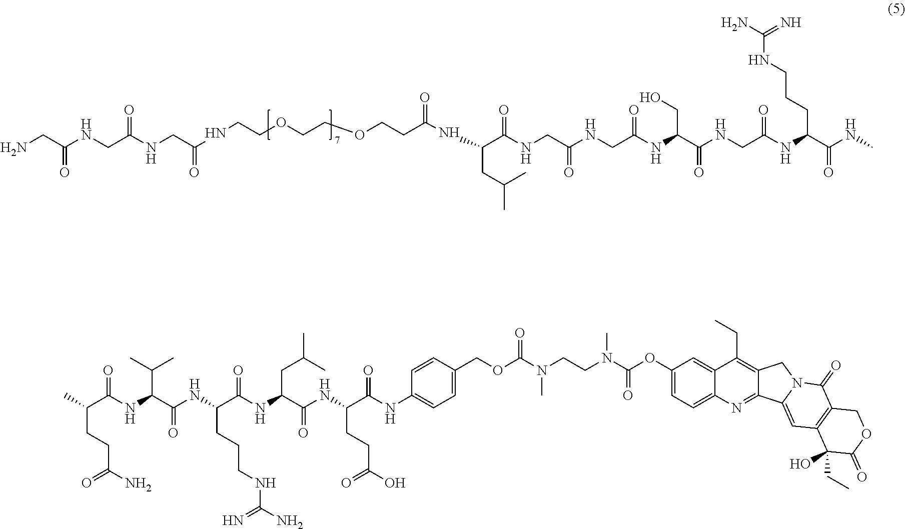

Compound of Formula (5) contains a linker that can be cleaved by uPA.

In some embodiments, the therapeutic agent is (Glycine)₃-amido-PEG₈-LGGSGRNAQVRLE-PAB-DMAE-α-hydroxy lactone linked SN38, shown as compound of Formula (6) below:

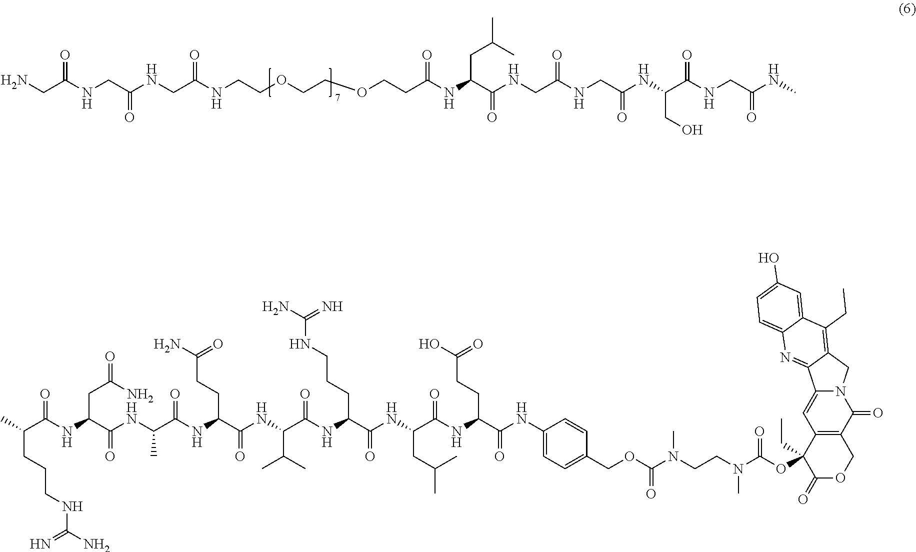

Compound of Formula 6 contains a linker that can be cleaved by uPA.

In some embodiments, the therapeutic agent is (Glycine)₃-amido-PEG₈-LGGSGRNAQVRLE-PAB-MMAE, shown as compound of Formula (7) below:

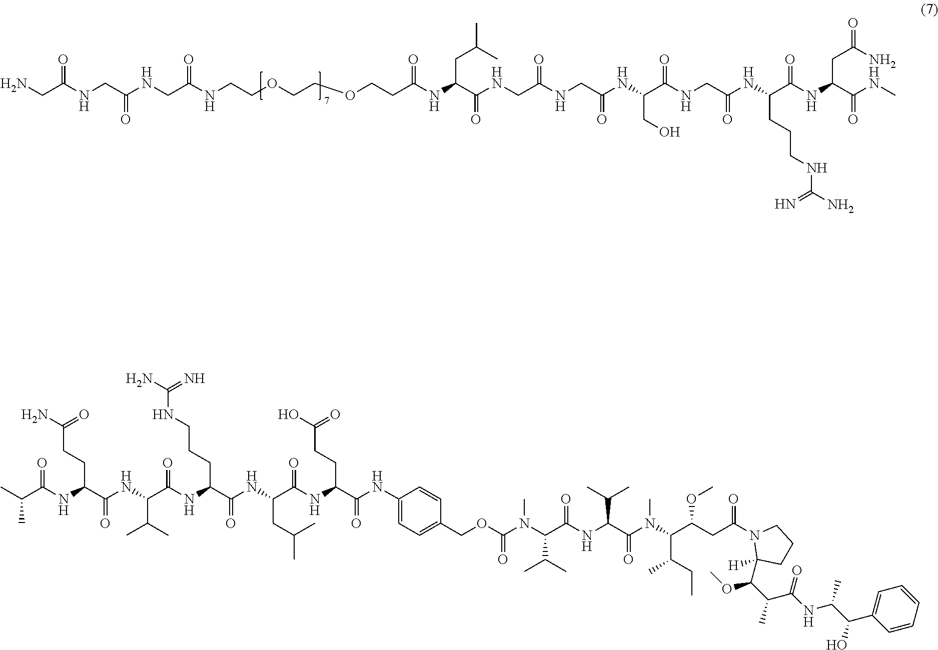

Compound of Formula (7) contains a linker that can be cleaved by uPA.

In some embodiments, the therapeutic agent is Glycine₃-amido-PEG₈-Val-Ala-PAB-DMAE-phenol linked SN38, wherein the linker is connected to position 10 of SN38 and has an 8-PEG spacer.

In some embodiments, the therapeutic agent is Glycine₃-amido-PEG₈-Val-Ala-PAB-DMAE-α-hydroxy lactone linked SN38, wherein the linker is connected to position 20 of SN38 and has an 8-PEG spacer.

In some embodiments, the therapeutic agent is (Glycine)₃-(PEG)₄-Val-Ala-PAB-MMAE, wherein the linker is connected to MMAE and has a 4-PEG spacer.

In some embodiments, the therapeutic agent is (Glycine)₃-(PEG)₈-Val-Ala-PAB-MMAE, wherein the linker is connected to MMAE and has an 8-PEG spacer.

Also provided are compounds having the structures of compounds of Formulae (1)-(7), and targeting conjugates (e.g., antibody drug conjugates) comprising these compounds.

In some embodiments, the targeting conjugate comprises about 1-80 therapeutic agents conjugated to the targeting moiety. In some embodiments, the targeting conjugate comprises about any one of 1-4, 1-5, 5-10, 4-10, 10-20, 1-20, 10-20, 20-40, or 40-80 therapeutic agents conjugated to the targeting moiety. In some embodiments, the targeting conjugate comprises at least about any one of 1, 2, 3, 4, 5, 6, 7, 8, 9, 10, 11, 12, 13, 14, 15, 16, 17, 18, 19, 20, 30, 40, 50, 60, 70, or 80 therapeutic agents. In some embodiments, the targeting conjugate comprises no more than about any one of 80, 70, 60, 50, 40, 30, 20, 19, 18, 17, 16, 15, 14, 13, 12, 11, 10, 9, 8, 7, 6, 5, 4, 3, 2 or 1 therapeutic agents.

### Oligonucleotide

In some embodiments, the effector molecule is an oligonucleotides. In some embodiments, the oligonucleotide is at least about any one of 2, 5, 10, 20, 30, 40, 50, 60, 70, 80, 90, 100, 120, 150, 200 or more nucleotides or base pairs long. In some embodiments, the oligonucleotide is no more than about any one of 200, 150, 120, 100, 90, 80, 70, 60, 50, 40, 30, 20, 10, 5 or 2 nucleotides or base pairs long. In some embodiments, the oligonucleotide is about any one of 2-10, 5-10, 10-20, 10-40, 10-60, 10-80, 10-100, 100-150, 150-200, 20-40, 40-80, 80-100, 100-200, 10-150, 10-200, 2-20, 2-50, or 2-100 nucleotides or base pairs long.

In some embodiments, the oligonucleotide is a DNA. In some embodiments, the oligonucleotide is a double-stranded DNA. In some embodiments, the oligonucleotide is a single-stranded DNA. In some embodiments, the oligonucleotide is a synthetic DNA.

In some embodiments, the oligonucleotide is an RNA. Exemplary RNAs includes, but are not limited to single-stranded RNA (ssRNA), double-stranded RNA (dsRNA), small interfering RNA (siRNA), small hairpin RNA (shRNA), or micro RNA. In some embodiments, the oligonucleotide is a synthetic RNA.

In some embodiments, the oligonucleotide is an immunomodulating polynucleotide. In some embodiments, the oligonucleotide is a pathogen-associated molecular pattern (PAMP) or other motif that can activate immune cells. PAMPs are molecules associated with various pathogens and are recognized by toll-like receptors (TLRs) and other pattern recognition receptors (PRRs) activating innate immune responses. The ability of PAMPs to recruit immune system in the absence of pathogens provides a strategy for treating a variety of diseases involving cell destruction (e.g., anticancer therapy) through the use of innate immune system response. One class of PAMPs that has been investigated for a variety of therapeutic applications is immunostimulating polynucleotides, such as CpG oligodeoxynucleotides (ODN) (e.g., agatolimod). It is thought that CpG ODNs mediate TLR9 dimerization in immune cells (e.g., B cells, monocytes, and plasmacytoid dendritic cells (pDCs)) to upregulate cytokines (e.g., type I interferon and interleukins), thereby activating natural killer cells. Exemplary PAMPs include, but are not limited to, CpG oligodeoxynucleotides (CpG-ODNs), herpes simplex virus (HSY) DNA, dsRNA, ssRNA. Further, the oligonucleotide may include one or more nucleic acid sequences that silence gene expression or induce intracellular death signaling, including, but not limited to, dsRNA, siRNA, shRNA, or micro RNA. In some embodiments, the oligonucleotide is selected from the group consisting of: GpT, GpG, GpA, ApA, ApT, ApG, CpT, CpA, CpG, TpA, TpT, and TpG oligonucleotides.

In some embodiments, the oligonucleotide is a CpG ODN. CpG ODNs are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides. In some embodiments, the CpG ODN has a modified phosphorothioate (PS) backbone, e.g., all nucleotides or a portion of the nucleotides in the CpG ODN has a PS backbone. When CpG motifs are unmethylated, they act as immunostimulants. CpG motifs are considered pathogen-associated molecular patterns (PAMPs) due to their abundance in microbial genomes but their rarity in vertebrate genomes. CpG-ODNs bind to and activate Toll-like receptor 9 (TLR9), initiating an innate immune response that supports the subsequent development of adaptive immunity. CpG-ODNs have been used as vaccine adjuvants to obtain desired immune modulation and synergistic immune response. CpG ODNs are generally divided into three classes: class A, class B, and class C. Class A CpG-ODNs typically contain poly-G tails with phosphorothioate backbones at 3′- and 5′-termini and a central palindromic sequence including a phosphate backbone. Class A CpG ODNs typically contain CpG within the central palindrome sequence. Class B CpG-ODNs typically include fully phosphorothioate backbone, and the sequence at the 5′ end of class B CpG ODN is often critical for TLR9 activation. Class C CpG-ODNs include fully phosphorothioate backbone with a 3′-end sequence enabling formation of a duplex. In some embodiments, the CpG-ODN is methylated. In some embodiments, the CpG-ODN is unmethylated.

In some embodiments, the targeting moiety is conjugated to an oligonucleotide comprising a CpG motif. In some embodiments, the targeting moiety is conjugated to an oligonucleotide comprising a plurality of CpG motifs. In some embodiments, the targeting moiety is conjugated to an oligonucleotide comprising multiple repeats of a CpG motif having the same sequence. In some embodiments, the targeting moiety is conjugated to multiple CpG-ODNs having different sequences.

In some embodiments, the CpG-ODN may exhibit stability (e.g., stability against nucleases) that is superior to that of CpG-ODNs containing mostly internucleoside phosphate (e.g., more than 50% of internucleoside phosphates) without substantially sacrificing their immunostimulating activity. This effect can be achieved, e.g., by incorporating at least 50% (e.g., at least 70%) internucleoside phosphorothioates or phosphorodithioates or through the inclusion of internucleoside phosphotriesters and/or internucleoside abasic spacers. Phosphotriesters and abasic spacers are also convenient for conjugation to a targeting moiety. Phosphate-based phosphotriesters and abasic spacers may also be used for reduction of off-target activity, relative to polynucleotides with fully phosphorothioate backbones. Without wishing to be bound by theory, this effect may be achieved by reducing self-delivery without disrupting targeting moiety-mediated delivery to target cells. Accordingly, an oligonucleotide can include about 15 or fewer contiguous internucleoside phosphorothioates (e.g., about 14 or fewer, about 13 or fewer, about 12 or fewer, about 11 or fewer, or about 10 or fewer contiguous internucleoside phosphorothioates). For example, a CpG-ODN containing a total of from about 12 to about 16 nucleosides may contain about 10 or fewer contiguous internucleoside phosphorothioates.

In some embodiments, the CpG ODN comprises one or more phosphorothioates (e.g., from about 1 to about 6 or from about 1 to about 4), e.g., at one or both termini (e.g., within the six 5′-terminal nucleosides or the six 3′-terminal nucleosides). The inclusion of one or more internucleoside phosphotriesters and/or phosphorothioates can enhance the stability of the polynucleotide by reducing the rate of exonuclease-mediated degradation.

In some embodiments, the CpG ODN further comprises one or more (e.g., from 1 to 6, from 1 to 12, from 1 to 18, from 1 to 24) auxiliary moieties (e.g., polyethylene glycols (PEGs)). The auxiliary moiety may be a part of a capping group, bioreversible group, or non-bioreversible group. The auxiliary moieties may be bonded to the linkers (e.g., to the linkers bonded to phosphates, phosphorothioates, or phosphorodithioates in the immunomodulating (e.g., immunostimulating) polynucleotides). Inclusion of the auxiliary moieties (e.g., PEGs) in the CpG ODN may improve pharmacokinetic and/or biodistribution properties of the targeting conjugate relative to a reference conjugate lacking such auxiliary moieties. In some embodiments, the CpG oligonucleotide is linked to a cleavable linker. In some embodiments, the CpG oligonucleotide and attached linker comprise a structure of 5′Amino Modifier-Spacer-Pₕ-CpG ODN (Formula IV), wherein the Spacer is (CH₂)ₙ-(PEG)ₘ, h, n and m are integers, h=0 or 1, n≥1, and m≥0, P is a cleavage site. In some embodiments, the CpG oligonucleotide and attached linker comprise a structure of 3′Amino Modifier-Spacer-Pₕ-CpG ODN (Formula V), wherein Spacer is (CH₂)ₙ-(PEG)ₘ, h, n and m are integers, h=0 or 1, n≥1, and m≥0, P is cleavage site. In some embodiments, the CpG oligonucleotide comprises a nucleic acid sequence selected from the group consisting of:

(SEQ ID NO: 66)
5′-TCGAACGTTCGAACGTTCGAACGTTCGAAT-3′
and
(SEQ ID NO: 67)
5′-T*C*G*A*A*C*G*T*T*C*G*A*A*C*G*T*T*
C*G*A*A*C*G*T*T*C*G*A*A*T*-3′,
wherein * represents a phosphorothioate linkage.

In some embodiments, the CpG oligonucleotide and its attached linker comprise a structure selected from the following:

(SEQ ID NO: 68)
5′ Amino Modifier-(CH₂)₁T*C*G*A*A*C*G*T*T*C*G
*A*A*C*G*T*T*C*G*A*A*C*G*T*T*C*G*A*A*T*-3′,
(SEQ ID NO: 69)
5′Amino Modifier-(CH₂)₁₂-thiol (CH₂)₆-S-S-T*C
*G*A*A*C*G*T*T*C*G*A*A*C*G*T*T*C*G*A*A*C*G*T*
T*C*G*A*A*T*-3′,
(SEQ ID NO: 70)
5′Amino Modifier-(CH₂)₆-(PEG)6-T*C*G*A*A*C*G*
T*T*C*G*A*A*C*G*T*T*C*G*A*A*C*G*T*T*C*G*A*A*
T*-3′,
(SEQ ID NO: 71)
5′ Amino Modifier-(CH₂)₁₂-(PEG)6-T*C*G*A*A*C*
G*T*T*C*G*A*A*C*G*T*T*C*G*A*A*C*G*T*T*C*G*A*A
*T*-3′,
(SEQ ID NO: 72)
5′Thiol Modifier-(CH₂)₆-S-S-T*C*G*A*A*C*G*T*T
*C*G*A*A*C*G*T*T*C*G*A*A*C*G*T*T*C*G*A*A*T*-3′,
(SEQ ID NO: 73)
5′- T*C*G*A*A*C*G*T*T*C*G*A*A*C*G*T*T*C*G*A*A
*C*G*T*T*C*G*A*A*T*-S-S-(CH₂)₆-3′Thiol Modifier,
and
Formula VIII:
(SEQ ID NO: 157)
5′Amino Modifier-LGGSGRNAQVRLEGSG (SEQ ID NO:
147)-(PEG)₁₂-T*C*G*A*A*C*G*T*T*C*G*A*A*C*G*
T*T*C*G*A*A*C*G*T*T*C*G*A*A*T,

wherein * represents a phosphorothioate linkage. In some embodiments, the CpG oligonucleotide has a nucleic acid sequence having at least 80% (e.g., at least about any one of 85%, 90%, 91%, 92%, 93%, 94%, 95%, 96%, 97%, or 99%; or 100%) sequence identity to a nucleic acid sequence of SEQ ID NO: 66 or 67. Also provided are CpG oligonucleotides comprising a nucleic acid sequence having at least 80% (e.g., at least about any one of 85%, 90%, 91%, 92%, 93%, 94%, 95%, 96%, 97%, or 99%; or 100%) sequence identity to a nucleic acid sequence of SEQ ID NO: 66 or 67.

In some embodiments, the oligonucleotide comprises a linker, such as a chemical linker or a peptide linker. In some embodiments, the linker is selected from the group consisting of SH₂-Spacer, MAL-Spacer, NH₂-Spacer, and Osu-Spacer. A spacer, as used herein, when present, links a targeting moiety to an effector molecule directly or indirectly. See, for example, US20100040637A1. In some embodiments, the linker is a poly-PEG linker.

### Detectable Label

In some embodiments, the effector molecule is a detectable label. As used herein, a label is a moiety that facilitates detection of the targeting moiety and/or facilitates detection of a molecule to which the targeting moiety binds. Non-limiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

For diagnostic purposes, the label may be a radionuclide, a radiological contrast agent, a paramagnetic ion, a metal, a fluorescent label, a chemiluminescent label, an ultrasound contrast agent and a photoactive agent. Such diagnostic labels are well known and any such known labels may be used.

In some embodiments, the detectable label is a radionuclide. “Radionuclides” are often referred to as “radioactive isotopes” or “radioisotopes.” Exemplary radionuclides or stable isotopes that may be attached to the targeting moieties described herein include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵, ¹³¹I, ¹⁵⁴⁻¹⁵⁸Gd, ³²P ¹¹C, ¹³N, ¹⁵O, ¹⁸⁶Re, ¹⁸⁸Re, ⁵¹Mn, ^{52m}Mn, Co, ⁷²As, ⁷⁵Br, ⁷⁶Br, ^{82m}Rb, ⁸³Sr, or other gamma-, beta-, or positron-emitters.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoelytherin, phycocyanin, allophycocyanin, ophthaldehyde and fluorescamine. Chemiluminescent labels of use may include luminol, isoluminol, an aromatic acridinium ester, an imidazole, an acridinium salt or an oxalate ester.

In some embodiments, the effector molecule comprises a chelating compound that chelates the radionuclide. In some embodiments, the chelating compound chelates a radioactive metal. In some embodiments, the chelating compound chelates a metal ¹⁸F. In some embodiments, the chelating compound is a hydrophilic chelating compound, which can bind metal ions and help to ensure rapid in vivo clearance. Particularly useful metal-chelating compound combinations include 2-benzyl-DTPA (diethylenetriamine pentaacetic acid) and its monomethyl and cyclohexyl analogs, used with diagnostic isotopes in the general energy range of 60 to 4,000 keV, such as ¹²¹I, ¹³¹I, ¹²³I, ¹²⁴I, ⁶²Cu, ⁶⁴Cu, ¹⁸F, ¹¹¹In, ⁶⁷Ga, ⁶⁸Ga, ⁹⁹Tc, ⁹⁴Tc, ¹¹C, ¹³N, ¹⁵O, ⁷⁶Br, for radio-imaging. The same chelating compounds, when complexed with nonradioactive metals, such as manganese, iron and gadolinium are useful for MRI. Macrocyclic chelating compounds such as NOTA (1,4,7-triazacyclononane-1,4,7-triacetic acid), DOTA (1,4,7,10-Tetraazacyclododecane-N,N′,N″,N′″-tetraacetic acid), TETA (bromoacetamido-benzyl-tetraethylaminetetraacetic acid) and NETA ({4-[2-(bis-carboxymethyl-amino)-ethyl]-7-carboxymethyl-[1,4,7]triazonan-1-yl}-acetic acid) are of use with a variety of diagnostic radiometals, such as gallium, yttrium and copper. Such metal-chelating complexes can be made very stable by tailoring the ring size to the metal of interest.

In some embodiments, the chelating compound comprises a functional group that can be conjugated to the targeting moiety. In some embodiments, the chelating compound comprises a functional group that is reactive with a primary amine (—NH₂) group in the targeting moiety. Exemplary functional groups that can be conjugated to a primary amine, e.g., a lysine side chain, of the targeting moiety, include, but are not limited to, isothiocyanates, isocyanates, acyl azides, N-hydroxysuccinimide (NHS) esters, sulfonyl chlorides, aldehydes, glyoxals, epoxides, oxiranes, carbonates, aryl halides, imidoesters, carbodiimides, anhydrides, and fluorophenyl esters. Most of these functional groups conjugate to amines by either acylation or alkylation.

In some embodiments, the chelating compound comprises a functional group that is reactive with a cysteine side chain (i.e., sulfhydryl group) in the targeting moiety. Exemplary sulfhydryl reactive groups include, but are not limited to, haloacetyls, maleimides, aziridines, acryloyls, arylating agents, vinylsulfones, pyridyl disulfides, TNB-thiols and disulfide reducing agents. Most of these groups conjugate to sulfhydryls by either alkylation (usually the formation of a thioether bond) or disulfide exchange (formation of a disulfide bond).

### C. Conjugation Site

In some embodiments, the targeting conjugate comprises one or more conjugation sites. The conjugation site allows covalent or non-covalent attachment of the effector molecule to the targeting moiety. The conjugation site may be part of the targeting moiety or present in a chemical moiety introduced to the targeting moiety. In some embodiments, the effector molecule is conjugated to a conjugation site disposed between a first targeting moiety and a second targeting moiety.

The conjugation sites may comprise one or more reactive groups for covalent conjugation. Common reactive groups in proteins include primary amines (—NH₂), carboxyls (—COOH), sulfhydryls (—SH) and carbonyls (—CHO, which can be created by oxidizing carbohydrate groups in glycoproteins).

In some embodiment, the conjugation site is within a polypeptide chain of the targeting moiety. In some embodiments, the conjugation site is at the side chain of an amino acid residue of the targeting moiety. In some embodiments, the conjugation site is at the N-terminus of a polypeptide chain of the targeting moiety. In some embodiments, the conjugation site is at the C-terminus of a polypeptide chain of the targeting moiety.

In some embodiments, the conjugation site is an endogenous conjugation site on the targeting moiety. In some embodiments, the conjugation site is an engineered conjugation site introduced into the targeting moiety. For example, the conjugation site may be present in a peptide, such as a tag, fused to the targeting moiety.

In some embodiments, the targeting moiety is bispecific or bivalent, wherein the targeting moiety comprises: (1) a conjugation moiety C comprising a conjugation site, and (2) two target-binding moieties A1 and A2, wherein A1 is a targeting peptide or an antibody or antigen-binding fragment thereof recognizing a first target molecule, and wherein A2 is a targeting peptide or an antibody or antigen-binding fragment thereof recognizing a second target molecule, and wherein A1 is fused to the N-terminus of C, and wherein A2 is fused to the C-terminus of C. In some embodiments, A1 and A2 are different. In some embodiments, A1 and A2 are the same.

In some embodiments, the conjugation site comprises a reactive thiol group, e.g., a cysteine residue of the targeting moiety.

In some embodiment, the conjugation site comprises a reactive amine group (“amine conjugation site”), e.g., a lysine or arginine residue of the targeting moiety. Primary amines exist at the N-terminus of each polypeptide chain and in the side chain of lysine and arginine (Lys, K) residues. These primary amines are positively charged at physiologic pH; therefore, they occur predominantly on the outside surfaces of native protein tertiary structures where they are readily accessible to conjugation reagents introduced into the aqueous medium. Furthermore, among the available functional groups in typical biological or protein samples, primary amines are especially nucleophilic; this makes them easy to target for conjugation with several reactive groups. One of the most specific and efficient reagents are those that use the N-hydroxysuccinimidyl ester (NHS ester) reactive group. In some embodiments, a sulfhydryl group can be introduced at the site of primary amines—especially those of lysine residues—by modification with reagents such as Traut's reagent (2-iminothiolane), MBS, SPDP, SATA, and their derivatives.

### Transglutaminase Conjugation Site

In some embodiments, the effector molecule (e.g., a therapeutic agent) is conjugated to a transglutaminase conjugation site in the targeting moiety. In some embodiments, the targeting moiety and the effector molecule are conjugated to each other via an isopeptide bond. In some embodiments, the targeting moiety comprises a transglutaminase conjugation site. In some embodiments, the targeting moiety and the effector molecule are conjugated to each other by a transglutamination reaction. In some embodiments, the transglutamination reaction is catalyzed by a transglutaminase.

Transglutaminase conjugation has been described in, for example, WO2019057772 and WO2012059882. Transglutaminases are protein-glutamine γ-glutamyltransferases, which typically catalyze pH-dependent transamidation of glutamine residues with lysine residues. The transglutaminase can be obtained or made from a variety of sources, or engineered to catalyze transamidation of one or more endogenous glutamine residues with one or more lysine residues or amine donor agents containing one or more reactive amines. Transglutaminases transfer the γ-glutaminyl of an acyl donor glutamine to an acyl acceptor amine group, such as primary amine or the F-amino group of lysine, resulting in an isopeptide bond connecting the acyl donor glutamine and the acyl acceptor residue. The isopeptide bond formed by transglutaminase catalysis is highly stable to proteases activity, and are not necessarily localized at N- and C-terminus. In some embodiments, the transglutaminase preferentially recognizes a peptide sequence that harbors an amino acid residue having an acyl acceptor group, and a peptide sequence that harbors an amino acid residue having an acyl donor group. The amino acid residue having the acyl acceptor group is referred herein as the “acyl acceptor residue” (e.g., lysine, N-terminal glycine) and the amino acid residue having the acyl donor group is referred herein as the “acyl donor residue” (e.g., glutamine).

In some embodiments, the acyl donor residue is an endogenous Gln. In some embodiments, the endogenous Gln sites are in the Fc region of the targeting moiety (e.g., antibody or antigen-binding fragment thereof). In some embodiments, the Gln in the Fc of the targeting moiety is deglycosylated. In some embodiments, the endogenous Gln sites are not in an Fc region. In some embodiments, there are more than one endogenous acyl donor Gln in the targeting moiety. In some embodiments, the endogenous Gln is at the N-terminus of the targeting moiety. In some embodiments, the endogenous Gln is at the C-terminus of the targeting moiety. In some embodiments, the endogenous Gln is at an internal position of the targeting moiety.

In some embodiments, the acyl donor residue is an engineered Gln residue introduced to the targeting moiety. In some embodiment, the non-endogenous Gln is incorporated into the targeting moiety through an amino acid modification, such as insertion or substitution. In some embodiments, the substitution comprises replacing a wild type amino acid with another (e.g., a non-wild type amino acid residue). In some embodiments, the insertion comprises inserting one or more amino acid(s) (e.g., inserting one, two, three or more amino acids).

In some embodiments, the non-endogenous Gln is in a tag. In some embodiments, the acyl donor glutamine-containing tag comprises at least one Gln. In some embodiments, the tag comprises multiple copies of the same sequence. In some embodiments, there are multiple Gln-containing tags linked to the targeting moiety. In some embodiments, the tag is linked to the targeting moiety through a peptide linker. In some embodiments, the peptide linker is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids long. In some embodiments, the tag may further comprise a purification tag. Exemplary purification tags include, but are not limited to, FLAG, GST, HA, poly-histidine, Myc, T7, AUl epitope, AU5 epitope, ABP, Streptavidin/Biotin, calmodulin binding peptide, MBP, chloramphenicol acetyl transferase, chitin binding domain, galactose-binding protein, EE-tag, histidine affinity tag, etc.

In some embodiments, the acyl donor glutamine-containing tag comprises an amino acid sequence (XᵣQₛXₜ-Lₚ)_{q} (Formula VI, SEQ ID NO:154), wherein r≥0, t≥0, s≥1, p=0 or 1, q≥1, L is a linker, X is any amino acid (e.g., conventional amino acid Leu, Ala, Gly, Ser, Val, Phe, Tyr, His, Arg, Asn, Glu, Asp, Cys, Gln, Ile, Met, Pro, Thr, Lys, or Trp or nonconventional amino acid). In some embodiments, the acyl donor glutamine-containing tag comprises an amino acid sequence (XQXX-Lₚ)_{q} (Formula VII, SEQ ID NO:155), wherein p=0 or 1, q≥1, L is an amino acid linker, X is any amino acid (e.g., conventional amino acid Leu, Ala, Gly, Ser, Val, Phe, Tyr, His, Arg, Asn, Glu, Asp, Cys, Gln, Ile, Met, Pro, Thr, Lys, or Trp or nonconventional amino acid). In some embodiments, the acyl donor glutamine-containing tag comprises an amino acid sequence selected from the group consisting of Q, LQG, LLQ, LQSP (SEQ ID NO:153), LLQGG (SEQ ID NO:13), LLQG (SEQ ID NO:14), GLLQG (SEQ ID NO:15), LSLSQG (SEQ ID NO:16), GGGLLQGG (SEQ ID NO:17), GSPLAQSHGG (SEQ ID NO:18), GLLQGGG (SEQ ID NO:19), GLLQGG (SEQ ID NO:20), GLLQ (SEQ ID NO:21), LLQLLQGA (SEQ ID NO:22), LLQGA (SEQ ID NO:23), LLQYQGA (SEQ ID NO:24), LLQGSG (SEQ ID NO:25), LLQYQG (SEQ ID NO:26), LLQLLQG (SEQ ID NO:27), SLLQG (SEQ ID NO:28), LLQLQ (SEQ ID NO:29), LLQLLQ (SEQ ID NO:30), LLQGR (SEQ ID NO:31), LLQGPP (SEQ ID NO:32), LLQGPA (SEQ ID NO:33), GGLLQGPP (SEQ ID NO:34), GGLLQGA (SEQ ID NO:35), LLQGPGK (SEQ ID NO:36), LLQGPG (SEQ ID NO:37), LLQGP (SEQ ID NO:38), LLQP (SEQ ID NO:39), LLQPGK (SEQ ID NO:40), LLQAPGK (SEQ ID NO:41), LLQGAPG (SEQ ID NO:42), LLQGAP (SEQ ID NO:43), LLQLQG (SEQ ID NO:44), QVQLKE (SEQ ID NO:45), VQLKE (SEQ ID NO:46), LQQP (SEQ ID NO:47), PQQF (SEQ ID NO:48), and GQQQL (SEQ ID NO:49), LLQGLLQGLLQG (SEQ ID NO:1), LLQGGSGLLQGGSGLLQG (SEQ ID NO:2), LQSPLQSPLQSP (SEQ ID NO:3), LQSPGSGLQSPGSGLQSP (SEQ ID NO:4), PNPQLPFPNPQLPFPNPQLPF (SEQ ID NO:5), PNPQLPFGSGPNPQLPFGSGPNPQLPF (SEQ ID NO:6), PKPQQFMPKPQQFMPKPQQFM (SEQ ID NO:7), PKPQQFMGSGPKPQQFMGSGPKPQQFM (SEQ ID NO:8), GQQQLGGQQQLGGQQQLG (SEQ ID NO:9), GQQQLGGSGGQQQLGGSGGQQQLG (SEQ ID NO:10), RLQQPRLQQPRLQQP (SEQ ID NO:11), RLQQPGSGRLQQPGSGRLQQP (SEQ ID NO:12).

In some embodiments, there is provided a transglutaminase conjugation peptide comprising an amino acid sequence selected from the group consisting of SEQ ID NOs: 1-12. In some embodiments, there is provided a targeting conjugate comprising a targeting moiety and an effector molecule, wherein the effector molecule is conjugated to the targeting moiety via a transglutaminase conjugation peptide comprising an amino acid sequence selected from the group consisting of SEQ ID NOs: 1-12.

In some embodiments, the Gln-containing tag is at the N-terminus of the targeting moiety. In some embodiments, the Gln-containing tag is at the C-terminus of the targeting moiety. In some embodiments, the Gln-containing tag is at an internal position of the targeting moiety.

In some embodiments, the targeting moiety comprises at least one endogenous glutamine residue made reactive in a transamidation reaction by antibody engineering. In some embodiments, the antibody engineering is antibody deglycosylation (e.g., enzymatic deglycosylation); or amino acid modification including amino acid deletion, insertion, substitution, mutation, or any combination thereof on the antibody. For example, the wild-type amino acid Asn (N) at position 297 in an antibody may be substituted or replaced with amino acid Ala (A), resulting in aglycosylation at position 297 and reactive endogenous glutamine (Q) at position 295. In another example, the amino acid modification in the antibody is an amino acid substitution from N to Q at position 297, resulting in aglycosylation at position 297, reactive endogenous Q at position 295, and site-specific conjugation between the N297Q and Q295 and one or more amine donor agents at these two sites in the presence of a transglutaminase. In some embodiments, the antibody can be engineered to remove glycosylation sites.

In some embodiments, the targeting moiety comprises an endogenous glutamine residue that is reactive for transglutaminase conjugation.

In some embodiments, the targeting moiety comprises a transglutaminase conjugation site having a plurality of glutamine residues that are reactive for conjugation to an amine group in the effector molecule. In some embodiments, the transglutaminase conjugation site comprises multiple glutamine-containing tag sequences in tandem, wherein a plurality (e.g., 2, 3, 4, 5, 6, or more) of effector molecules are conjugated to the transglutaminase conjugation site.

### Oligonucleotide Binding Polypeptide (OBP)

In some embodiments, an oligonucleotide is conjugated non-covalently to the targeting moiety via an oligonucleotide binding polypeptide introduced to the targeting moiety. In some embodiments, the oligonucleotide is conjugated to the oligonucleotide binding polypeptide through charge-charge interactions. In some embodiments, the oligonucleotide binding polypeptide is a cationic peptide. In some embodiments, the oligonucleotide binding polypeptide is a neutrally charged peptide.

Many nucleic acid binding proteins use short peptide sequences to provide specificity in recognizing their targets, which may either have a specific sequence or a specific conformation. Peptides containing alternating lysine have been shown to bind to poly(dG-d5meC) in the Z conformation, and stabilize the higher energy form. See, for example, H. Takeuchi et al., (1991) FEBS Lett., 279, 253-255 and H. Takeuchi et al., (1994) J. Mol. Biol., 236, 610-617.

In some embodiments, the CpG binding polypeptide is positively charged. In some embodiments, the CpG binding polypeptide comprises one or more positively charged amino acid residues. In some embodiments, the CpG binding polypeptide comprises polar amino acid residues. In some embodiments, the CpG binding polypeptide comprises short motifs comprising multiple positively charged amino acid residues. In some embodiments, the short motif comprises tandem positively charged amino acid residues. In some embodiments, the CpG binding peptide further comprises modifications at the N terminus. In some embodiments, the CpG binding peptide comprises an aminobenzoic acid at the N terminus.

In some embodiments, the CpG binding polypeptide is selected from the group consisting of RSQSRSRYYRQRQRSRRRRRRS (SEQ ID NO:56); RRRLHRIIRRQHRSCRRRKRR (SEQ ID NO:57); MPRRRRSSSRPVRRRRRPRVSRRRRRRGGRRRR (SEQ ID NO:58); KKSAKKTPKKAKKPKKSAKKTPKKAKKP (SEQ ID NO:59); AKKAKSPKKAKAAKPKKAPKSPAKAK (SEQ ID NO:60); MRRAHHRRRRASHRRMRGG (SEQ ID NO:61); KHKHKHKHKKKHKHKHKHKKKHKHKHKHKK (SEQ ID NO:62); KGKGKGKGKKKGKGKGKGKKKGKGKGKGKK (SEQ ID NO:63); KKALLALALHHLAHLALHLALALKKA (SEQ ID NO:64); and YSPTSPSYSPTSPSYSPTSPSY (SEQ ID NO:65). Also provided are CpG binding polypeptides comprising an amino acid sequence selected from the group consisting of SEQ ID NOs: 56-65. In some embodiments, there is provided a targeting conjugate comprising a targeting moiety, a CpG oligonucleotide, and a CpG binding polypeptide, wherein the CpG oligonucleotide is conjugated to the targeting moiety via the CpG binding polypeptides, and wherein the CpG binding polypeptide comprises an amino acid sequence selected from the group consisting of SEQ ID NOs: 56-65.

In some embodiments, the CpG binding polypeptide is fused to the targeting moiety. In some embodiments, the CpG binding polypeptide is fused to the N terminus of one or more of the polypeptide chains of the targeting moiety. In some embodiments, the CpG binding polypeptide is fused to the C terminus of one or more of the polypeptide chains of the targeting moiety. In some embodiments, the CpG binding polypeptide is fused to an internal position of one or more of the polypeptide chains of the targeting moiety. In some embodiments, wherein the targeting moiety is an antibody or antigen-binding fragment, the CpG binding polypeptide is fused to the C terminus of a heavy chain of the antibody or antigen-binding fragment, e.g., via a single peptide bond or a linker. In some embodiments, wherein the targeting moiety is an antibody or antigen-binding fragment, the CpG binding polypeptide is fused to the C terminus of a light chain of the antibody or antigen-binding fragment, e.g., via a single peptide bond or a peptide linker.

### D. Cleavage Site

In some embodiments, the targeting conjugate comprises one or more cleavage sites. In some embodiments, the targeting conjugate comprises one or more cleavage sites in or linked to the targeting moiety. In some embodiments, the targeting moiety does not comprise one or more cleavage sites. In some embodiments, the targeting conjugate comprises one or more cleavage sites in or linked to the effector molecule. In some embodiments, the targeting conjugate comprises one or more cleavage sites in or linked to the oligonucleotide binding polypeptide.

In some embodiments, the one or more cleavage sites in the targeting conjugate are cleaved at a target site, e.g., a diseased site. In some embodiments, the cleavage is triggered by a condition at the target site, such as a protease, a change in pH, redox level, hypoxia, oxidative stress, hyperthermia, and/or extracellular ATP concentration. In some embodiments, the one or more cleavage sites in the targeting conjugate are not cleaved at a non-target site, such as in normal or healthy tissues. In some embodiments, the condition that triggers cleavage of the cleavage site at the target site is not found at non-target sites. In some embodiments, the condition that triggers cleavage of the cleavage site is present at the target site at a level that is at least about any one of 1.5, 2, 5, 10, 20, 50, 100, 200, 500, 1000 times or higher than the condition at non-target sites. For example, a protease (e.g., uPA) that cleaves the cleavage site is present at a target site at a concentration that is at least about any one of 1.5, 2, 5, 10, 20, 50, 100, 200, 500, 1000 times or higher than the concentration of the protease at non-target sites. In some embodiments, the extracellular ATP concentration at a target site is at least about any one of 1.5, 2, 5, 10, 20, 50, 100, 200, 500, 1000 times or higher than the extracellular ATP concentration at non-target sites. In some embodiments, the pH at a target site is at least about any one of 0.1, 0.2, 0.3, 0.4, 0.5, 0.6, 0.7, 0.8, 0.9, 1, 1.5, 2, 2.5, or 3 lower than the pH at non-target sites. In some embodiments, the pH at a target site is at least about any one of 0.1, 0.2, 0.3, 0.4, 0.5, 0.6, 0.7, 0.8, 0.9, 1, 1.5, 2, 2.5, or 3 higher than the pH at non-target sites.

In some embodiments, the cleavage site is a protease cleavage site. In some embodiments, the cleavage site is cleavable by a disease-specific protease, such as a tumor-specific protease. In some embodiments, the tumor-specific protease express at elevated levels in diseased tissues, such as tumor tissues, compared to normal tissues. For example, studies have shown that tumor tissues exhibit increased activity of specific proteases and decreased activity of the opposing endogenous inhibitors (Sevenich L and Joyce J A. Genes & Dev. 2014. 28: 2331-2347. 2014). In some embodiments, tumor-specific protease is selected from the group consisting of matriptase (MTSP1), urinary-type plasminogen activator (uPA), legumain, PSA (also called KLK3, kallikrein-related peptidase-3), matrix metalloproteinase-2 (MMP-2), matrix metalloproteinase-9 (MMP9), human neutrophil elastase (HNE), proteinase 3 (Pr3), cathepsin B and cathepsin K. In some embodiments, the cleavage occurs at a tumor site, for example, in a tumor microenvironment. In some embodiments, the cleavage occurs outside of a cell, such as outside of the tumor cells in a tumor microenvironment.

In some embodiments, the cleavage site is a substrate for an enzyme selected from the group consisting of legumain, plasmin, TMPRSS-3/4, MMP-9, MTl-MMP, cathepsin, caspase, human neutrophil elastase, beta-betasecretase, uPA, and PSA.

In some embodiments, the cleavage site is a uPA substrate peptide. Urokinase plasminogen activator (uPA) is a serine protease that is part of the urokinase plasminogen activating system (uPAS). The uPA converts the proenzyme plasminogen in the serine protease plasmin, involved in a number of physiopathological processes requiring basement membrane (BM) and/or extracellular matrix (ECM) remodeling, including tumor progression and metastasis. In some embodiments, the uPA cleavage site comprises an amino acid sequence selected from the group consisting of LSGRSDNH (SEQ ID NO: 50), SGRSA (SEQ ID NO: 51), LGGSGRSANAILE (SEQ ID NO: 52), LGGSGRNAQVRLE (SEQ ID NO: 53), GSGRNAQV (SEQ ID NO: 54), and SGR (SEQ ID NO: 55). Also provided are uPA cleavage peptides comprising an amino acid sequence selected from the group consisting of SEQ ID NOs: 50-55.

In some embodiments, the targeting conjugate comprises a single peptide substrate cleavable by a disease-specific protease, such as uPA, e.g., any one of SEQ ID NOs: 50-55. In some embodiments, the targeting conjugate comprises two or more copies (such as any one of 2, 3, 4, 5, or more) of a peptide substrate cleavable by a disease-specific protease. In some embodiments, the targeting conjugate comprises a peptide substrate that can be cleaved by more than one (e.g., any of 2, 3, 4, or more) disease-specific proteases. In some embodiments, the targeting conjugate comprises two or more (e.g., any of 2, 3, 4, or more) peptide substrates cleavable by one or more (e.g., any of 2, 3, 4, or more) disease-specific proteases. Any one of the protease peptide substrate sequences disclosed herein (e.g., SEQ ID NOs: 50-55) can be mixed and matched to provide a disease-sensing releasable moiety with optimal mechanism and dynamics for release of the effector molecules at a target (e.g., disease) site. The different protease substrate sequences or copies thereof can be fused to each other via peptide linkers to provide suitable cleavage sites.

### E. Exemplary Targeting Conjugates

In some embodiments, there is provided a targeting conjugate comprising: a targeting moiety, one or more effector molecules conjugated to the targeting moiety via a conjugation site. In some embodiments, each effector molecule is conjugated to the conjugation site via one or more linkers, and/or one or more protease cleavage sites. In some embodiments, the effector molecule is covalently conjugated to the targeting moiety. In some embodiments, the effector molecule is non-covalently conjugated to the targeting moiety. In some embodiments, the targeting moiety comprises two or more polypeptide chains. In some embodiments, the targeting moiety comprises a protease cleavage site. In some embodiments, the targeting conjugate comprises a first targeting moiety, a second targeting moiety, and a conjugation site linking the first targeting moiety and the second targeting moiety, wherein the one or more effector molecules are conjugated to the conjugation site. In some embodiments, the protease cleavage occurs outside of a cell. In some embodiments, the targeting conjugate has the structure of Formula I.

In some embodiments, there is provided a targeting conjugate comprising: (A) a first polypeptide comprising from the N-terminus to the C-terminus: a first antibody heavy chain, a first protease cleavage site (e.g., a uPA cleavage site), and a first conjugation site; (B) a second polypeptide comprising from the N-terminus to the C-terminus: a second antibody heavy chain, a second protease cleavage site (e.g., a uPA cleavage site), and a second conjugation site; (C) a first antibody light chain; (D) a second antibody light chain; (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first effector molecule is conjugated to the first conjugation site via the first linker; wherein the second effector molecule is conjugated to the second conjugation site via the second linker; wherein upon cleavage of the first protease cleavage site, the first effector molecule is released from the targeting conjugate; and wherein upon cleavage of the second protease cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 1A. An exemplary targeting conjugate may comprise: a targeting moiety comprising an antibody heavy chain comprising SEQ ID NO: 112 and an antibody light chain comprising SEQ ID NO: 113; and a therapeutic agent selected from the group consisting of compound of Formula (1), compound of Formula (2), compound of Formula (3), and compound of Formula (4).

In some embodiments, there is provided a targeting conjugate comprising: (A) a first polypeptide comprising from the N-terminus to the C-terminus: a first antibody heavy chain, a first protease cleavage site (e.g., a uPA cleavage site), a first conjugation site, and a first antigen-binding fragment (e.g., an scFv, scFab or nanobody); (B) a second polypeptide comprising from the N-terminus to the C-terminus: a second antibody heavy chain, a second protease cleavage site (e.g., a uPA cleavage site), a second conjugation site, and second antigen-binding fragment (e.g., an scFv, scFab or nanobody); (C) a first antibody light chain; (D) a second antibody light chain, (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first effector molecule and the first antigen-binding fragment are released from the targeting conjugate; and wherein upon cleavage of the second protease cleavage site, the second effector molecule and the second antigen-binding fragment are released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 1B. An exemplary targeting conjugate may comprise: a targeting moiety comprising an antibody heavy chain comprising SEQ ID NO: 124 and an antibody light chain of SEQ ID NO: 125; and a therapeutic agent selected from the group consisting of compound of Formula (1), compound of Formula (2), compound of Formula (3), and compound of Formula (4).

In some embodiments, there is provided a targeting conjugate comprising: (A) a first polypeptide comprising from the N-terminus to the C-terminus: a first antibody heavy chain, a first protease cleavage site, a first conjugation site, a second protease cleavage site, and a first antigen-binding fragment (e.g., scFv/scFab/nanobody); (B) a second polypeptide comprising from the N-terminus to the C-terminus: a second antibody heavy chain, a third protease cleavage site, a second conjugation site, a fourth protease cleavage site, and a second antigen-binding fragment (e.g., scFv/scFab/nanobody); (C) a first antibody light chain; (D) a second antibody light chain, (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first effector molecule and the first antigen-binding fragment are released from the targeting conjugate; wherein upon cleavage of the second protease cleavage site, the first antigen-binding fragment is released from the targeting conjugate; wherein upon cleavage of the third protease cleavage site, the second effector molecule and the second antigen-binding fragment are released from the targeting conjugate; and wherein upon cleavage of the fourth protease cleavage site, the second antigen-binding fragment is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 1C.

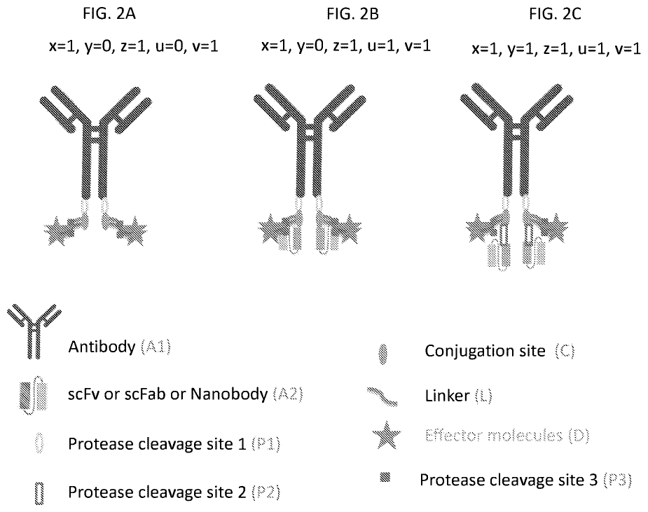

In some embodiments, there is provided a targeting conjugate comprising: (A) a first polypeptide comprising from the N-terminus to the C-terminus: a first antibody heavy chain, a first protease cleavage site (e.g., a uPA cleavage site), and a first conjugation site; (B) a second polypeptide comprising from the N-terminus to the C-terminus: a second antibody heavy chain, a second protease cleavage site (e.g., a uPA cleavage site), and a second conjugation site; (C) a first antibody light chain; (D) a second antibody light chain; (E) a first effector molecule (e.g., a small molecule drug) comprising a first linker and a third cleavage site; (F) a second effector molecule (e.g., a small molecule drug) comprising a second linker and a fourth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first effector molecule is conjugated to the first conjugation site via the first linker; wherein the second effector molecule is conjugated to the second conjugation site via the second linker; wherein upon cleavage of the first and/or the third protease cleavage sites, the first effector molecule is released from the targeting conjugate; and wherein upon cleavage of the second and/or the fourth protease cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 2A. An exemplary targeting conjugate may comprise: a targeting moiety comprising an antibody heavy chain comprising SEQ ID NO: 112 and an antibody light chain comprising SEQ ID NO: 113; and a therapeutic agent selected from the group consisting of compound of Formula (1), compound of Formula (2), compound of Formula (3), and compound of Formula (4). An exemplary targeting conjugate may comprise: a targeting moiety comprising an antibody heavy chain comprising SEQ ID NO: 112 and an antibody light chain comprising SEQ ID NO: 113; and a therapeutic agent selected from the group consisting of compound of Formula (5), compound of Formula (6), and compound of Formula (7).

In some embodiments, there is provided a targeting conjugate comprising: (A) a first polypeptide comprising from the N-terminus to the C-terminus: a first antibody heavy chain, a first protease cleavage site (e.g., a uPA cleavage site), a first conjugation site, and a first antigen-binding fragment (e.g., an scFv, scFab or nanobody); (B) a second polypeptide comprising from the N-terminus to the C-terminus: a second antibody heavy chain, a second protease cleavage site (e.g., a uPA cleavage site), a second conjugation site, and second antigen-binding fragment (e.g., an scFv, scFab or nanobody); (C) a first antibody light chain; (D) a second antibody light chain, (E) a first effector molecule (e.g., a small molecule drug) comprising a first linker and a third cleavage site; (F) a second effector molecule (e.g., a small molecule drug) comprising a second linker and a fourth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first effector molecule and the first antigen-binding fragment are released from the targeting conjugate; wherein upon cleavage of the second protease cleavage site, the second effector molecule and the second antigen-binding fragment are released from the targeting conjugate; wherein upon cleavage of the third cleavage site, the first effector molecule is released from the targeting conjugate; and wherein upon cleavage of the fourth cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 2B. An exemplary targeting conjugate may comprise: a targeting moiety comprising an antibody heavy chain comprising SEQ ID NO: 124 and an antibody light chain of SEQ ID NO: 125; and a therapeutic agent selected from the group consisting of compound of Formula (1), compound of Formula (2), compound of Formula (3), and compound of Formula (4). An exemplary targeting conjugate may comprise: a targeting moiety comprising an antibody heavy chain comprising SEQ ID NO: 124 and an antibody light chain of SEQ ID NO: 125; and a therapeutic agent selected from the group consisting of compound of Formula (5), compound of Formula (6), and compound of Formula (7).

In some embodiments, there is provided a targeting conjugate comprising: (A) a first polypeptide comprising from the N-terminus to the C-terminus: a first antibody heavy chain, a first protease cleavage site, a first conjugation site, a second protease cleavage site, and a first antigen-binding fragment (e.g., scFv/scFab/nanobody); (B) a second polypeptide comprising from the N-terminus to the C-terminus: a second antibody heavy chain, a third protease cleavage site, a second conjugation site, a fourth protease cleavage site, and a second antigen-binding fragment (e.g., scFv/scFab/nanobody); (C) a first antibody light chain; (D) a second antibody light chain, (E) a first effector molecule (e.g., a small molecule drug) comprising a first linker and a fifth cleavage site; (F) a second effector molecule (e.g., a small molecule drug) comprising a second linker and a sixth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first effector molecule and the first antigen-binding fragment are released from the targeting conjugate; wherein upon cleavage of the second protease cleavage site, the first antigen-binding fragment is released from the targeting conjugate; wherein upon cleavage of the third protease cleavage site, the second effector molecule and the second antigen-binding fragment are released from the targeting conjugate; wherein upon cleavage of the fourth protease cleavage site, the second antigen-binding fragment is released from the targeting conjugate; wherein upon cleavage of the fifth protease cleavage site, the first effector molecule is released from the targeting conjugate; wherein upon cleavage of the sixth cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 2C.

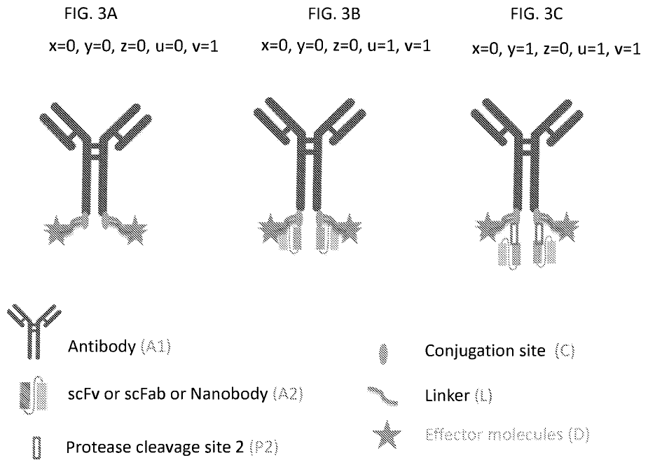

In some embodiments, there is provided a targeting conjugate comprising: (A) a first polypeptide comprising from the N-terminus to the C-terminus: a first antibody heavy chain and a first conjugation site; (B) a second polypeptide comprising from the N-terminus to the C-terminus: a second antibody heavy chain and a second conjugation site; (C) a first antibody light chain; (D) a second antibody light chain; (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first effector molecule is conjugated to the first conjugation site via the first linker; wherein the second effector molecule is conjugated to the second conjugation site via the second linker. In some embodiments, two or more effector molecules are conjugated to the first conjugation site. In some embodiments, two or more effector molecules are conjugated to the second conjugation site. An exemplary targeting conjugate is shown in FIG. 3A. An exemplary targeting conjugate may comprise: a targeting moiety comprising an antibody heavy chain comprising SEQ ID NO: 104 and an antibody light chain comprising SEQ ID NO: 105; and a therapeutic agent selected from the group consisting of selected from the group consisting of compound of Formula (1), compound of Formula (2), compound of Formula (3), and compound of Formula (4).

In some embodiments, there is provided a targeting conjugate comprising: (A) a first polypeptide comprising from the N-terminus to the C-terminus: a first antibody heavy chain, a first conjugation site, and a first antigen-binding fragment (e.g., an scFv, scFab, or nanobody); (B) a second polypeptide comprising from the N-terminus to the C-terminus: a second antibody heavy chain, a second conjugation site, and a second antigen-binding fragment (e.g., an scFv, scFab, or nanobody); (C) a first antibody light chain; (D) a second antibody light chain, (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker. In some embodiments, two or more effector molecules are conjugated to the first conjugation site. In some embodiments, two or more effector molecules are conjugated to the second conjugation site. An exemplary targeting conjugate is shown in FIG. 3B. An exemplary targeting conjugate may comprise: a targeting moiety comprising an antibody heavy chain comprising SEQ ID NO: 114 or 122 and an antibody light chain comprising SEQ ID NO: 115 or 123; and a therapeutic agent selected from the group consisting of Formula (1), compound of Formula (2), compound of Formula (3), and compound of Formula (4).

In some embodiments, there is provided a targeting conjugate comprising: (A) a first polypeptide comprising from the N-terminus to the C-terminus: a first antibody heavy chain, a first conjugation site, a first protease cleavage site, a first antigen-binding fragment (e.g., an scFv, scFab, or nanobody); (B) a second polypeptide comprising from the N-terminus to the C-terminus: a second antibody heavy chain, a second conjugation site, a second protease cleavage site, and a second antigen-binding fragment (e.g., an scFv, scFab, or nanobody); (C) a first antibody light chain; (D) a second antibody light chain, (E) a first effector molecule (e.g., a small molecule drug) comprising a first linker; (F) a second effector molecule (e.g., a small molecule drug) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first antigen-binding fragment is released from the targeting conjugate; and wherein upon cleavage of the second protease cleavage site, the second antigen-binding fragment is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 3C.

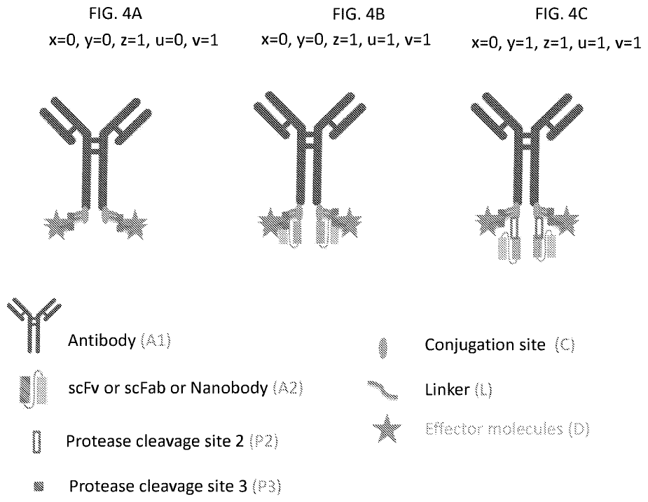

In some embodiments, there is provided a targeting conjugate comprising: (A) a first polypeptide comprising from the N-terminus to the C-terminus: a first antibody heavy chain and a first conjugation site; (B) a second polypeptide comprising from the N-terminus to the C-terminus: a second antibody heavy chain and a second conjugation site; (C) a first antibody light chain; (D) a second antibody light chain; (E) a first effector molecule (e.g., a small molecule drug) comprising a first linker and a first cleavage site; (F) a second effector molecule (e.g., a small molecule drug) comprising a second linker and a second cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first effector molecule is conjugated to the first conjugation site via the first linker; wherein the second effector molecule is conjugated to the second conjugation site via the second linker; wherein upon cleavage of the first cleavage site, the first effector molecule is released from the targeting conjugate; and wherein upon cleavage of the second cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 4A. An exemplary targeting conjugate may comprise: a targeting moiety comprising an antibody heavy chain comprising SEQ ID NO: 104 and an antibody light chain comprising SEQ ID NO: 105; and a therapeutic agent selected from the group consisting of compound of Formula (1), compound of Formula (2), compound of Formula (3), and compound of Formula (4). An exemplary targeting conjugate may comprise: a targeting moiety comprising an antibody heavy chain comprising SEQ ID NO: 104 and an antibody light chain comprising SEQ ID NO: 105; and a therapeutic agent selected from the group consisting of compound of Formula (5), compound of Formula (6), and compound of Formula (7).

In some embodiments, there is provided a targeting conjugate comprising: (A) a first polypeptide comprising from the N-terminus to the C-terminus: a first antibody heavy chain and a first antigen-binding fragment (e.g., an scFv, scFab, or nanobody); (B) a second polypeptide comprising from the N-terminus to the C-terminus: a second antibody heavy chain and a second antigen-binding fragment (e.g., an scFv, scFab, or nanobody); (C) a first antibody light chain; (D) a second antibody light chain, (E) a first effector molecule (e.g., a small molecule drug) comprising a first linker and a first cleavage site; (F) a second effector molecule (e.g., a small molecule drug) comprising a second linker and a second cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first cleavage site, the first effector molecule is released from the targeting conjugate; wherein upon cleavage of the second cleavage site, the second effector molecules is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 4B. An exemplary targeting conjugate may comprise: a targeting moiety comprising an antibody heavy chain comprising SEQ ID NO: 114 or 122 and an antibody light chain comprising SEQ ID NO: 115 or 123; and a therapeutic agent selected from the group consisting of compound of Formula (1), compound of Formula (2), compound of Formula (3), and compound of Formula (4). An exemplary targeting conjugate may comprise: a targeting moiety comprising an antibody heavy chain comprising SEQ ID NO: 114 or 122 and an antibody light chain comprising SEQ ID NO: 115 or 123; and a therapeutic agent selected from the group consisting of compound of Formula (5), compound of Formula (6), and compound of Formula (7).

In some embodiments, there is provided a targeting conjugate comprising: (A) a first polypeptide comprising from the N-terminus to the C-terminus: a first antibody heavy chain, a first conjugation site, a first protease cleavage site, a first antigen-binding fragment (e.g., an scFv, scFab, or nanobody); (B) a second polypeptide comprising from the N-terminus to the C-terminus: a second antibody heavy chain, a second conjugation site, a second protease cleavage site, and a second antigen-binding fragment (e.g., an scFv, scFab, or nanobody); (C) a first antibody light chain; (D) a second antibody light chain, (E) a first effector molecule (e.g., a small molecule drug) comprising a first linker and a third cleavage site; (F) a second effector molecule (e.g., a small molecule drug) comprising a second linker and a fourth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first effector molecule is conjugated to the first conjugation site via the first linker; wherein the second effector molecule is conjugated to the second conjugation site via the second linker; wherein upon cleavage of the first protease cleavage site, the first antigen-binding fragment is released from the targeting conjugate; wherein upon cleavage of the second protease cleavage site, the second antigen-binding fragment is released from the targeting conjugate; wherein upon cleavage of the third cleavage site, the first effector molecule is released from the targeting conjugate; wherein upon cleavage of the fourth cleavage site, the second effector molecule is released from the targeting conjugate. In some embodiments, two or more effector molecules are conjugated to the first conjugation site. In some embodiments, two or more effector molecules are conjugated to the second conjugation site. An exemplary targeting conjugate is shown in FIG. 4C.

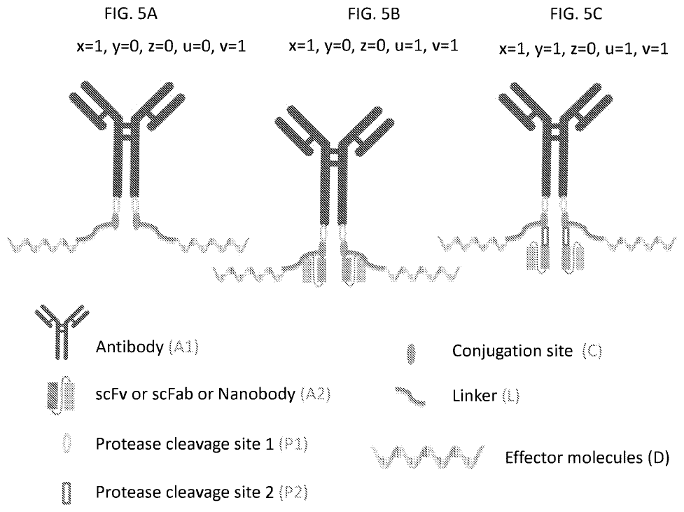

In some embodiments, there is provided a targeting conjugate comprising: (A) a first polypeptide comprising from the N-terminus to the C-terminus: a first antibody heavy chain, a first protease cleavage site (e.g., a uPA cleavage site), and a first conjugation site; (B) a second polypeptide comprising from the N-terminus to the C-terminus: a second antibody heavy chain, a second protease cleavage site (e.g., a uPA cleavage site), and a second conjugation site; (C) a first antibody light chain; (D) a second antibody light chain; (E) a first oligonucleotide (e.g., a CpG oligonucleotide) comprising a first linker; (F) a second oligonucleotide (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide is released from the targeting conjugate; and wherein upon cleavage of the second protease cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 5A. An exemplary targeting conjugate may comprise: a targeting moiety comprising an antibody heavy chain comprising SEQ ID NO: 112 and an antibody light chain comprising SEQ ID NO: 113; and a CpG ODN comprising the nucleic acid sequence selected from the group consisting of SEQ ID NOs: 68-73.

In some embodiments, there is provided a targeting conjugate comprising: (A) a first polypeptide comprising from the N-terminus to the C-terminus: a first antibody heavy chain, a first protease cleavage site (e.g., a uPA cleavage site), a first conjugation site, and a first antigen-binding fragment (e.g., an scFv, scFab or nanobody); (B) a second polypeptide comprising from the N-terminus to the C-terminus: a second antibody heavy chain, a second protease cleavage site (e.g., a uPA cleavage site), a second conjugation site, and second antigen-binding fragment (e.g., an scFv, scFab or nanobody); (C) a first antibody light chain; (D) a second antibody light chain, (E) a first oligonucleotide (e.g., a CpG oligonucleotide) comprising a first linker; (F) a second oligonucleotide (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide and the first antigen-binding fragment are released from the targeting conjugate; and wherein upon cleavage of the second protease cleavage site, the second oligonucleotide and the second antigen-binding fragment are released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 5B. An exemplary targeting conjugate may comprise: a targeting moiety comprising an antibody heavy chain comprising SEQ ID NO: 124 and an antibody light chain comprising SEQ ID NO: 125 and a CpG ODN comprising the nucleic acid sequence selected from the group consisting of SEQ ID NOs: 68-73.

In some embodiments, there is provided a targeting conjugate comprising: (A) a first polypeptide comprising from the N-terminus to the C-terminus: a first antibody heavy chain, a first protease cleavage site, a first conjugation site, a second protease cleavage site, and a first antigen-binding fragment (e.g., an scFv, scFab or nanobody); (B) a second polypeptide comprising from the N-terminus to the C-terminus: a second antibody heavy chain, a third protease cleavage site, a second conjugation site, a fourth protease cleavage site, and a second antigen-binding fragment (e.g., an scFv, scFab or nanobody); (C) a first antibody light chain; (D) a second antibody light chain, (E) a first oligonucleotide (e.g., a CpG oligonucleotide) comprising a first linker; (F) a second oligonucleotide (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide and the first antigen-binding fragment are released from the targeting conjugate; wherein upon cleavage of the second protease cleavage site, the first antigen-binding fragment is released from the targeting conjugate; wherein upon cleavage of the third protease cleavage site, the second oligonucleotide and the second antigen-binding fragment are released from the targeting conjugate; and wherein upon cleavage of the fourth protease cleavage site, the second antigen-binding fragment is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 5C.

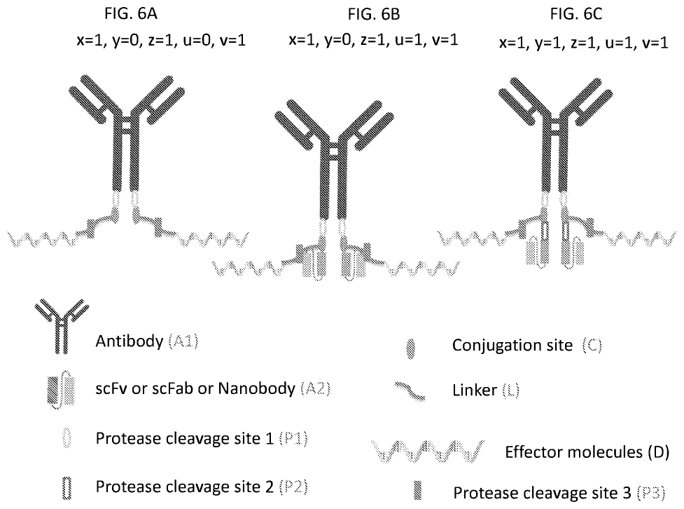

In some embodiments, there is provided a targeting conjugate comprising: (A) a first polypeptide comprising from the N-terminus to the C-terminus: a first antibody heavy chain, a first protease cleavage site (e.g., a uPA cleavage site), and a first conjugation site; (B) a second polypeptide comprising from the N-terminus to the C-terminus: a second antibody heavy chain, a second protease cleavage site (e.g., a uPA cleavage site), and a second conjugation site; (C) a first antibody light chain; (D) a second antibody light chain; (E) a first oligonucleotide (e.g., a CpG oligonucleotide) comprising a first linker and a third cleavage site; (F) a second oligonucleotide (e.g., a CpG oligonucleotide) comprising a second linker and a fourth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide is released from the targeting conjugate; wherein upon cleavage of the second protease cleavage site, the second oligonucleotide is released from the targeting conjugate; wherein upon cleavage of the third cleavage site, the first oligonucleotide is released from the targeting conjugate; and wherein upon cleavage of the fourth cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 6A. An exemplary targeting conjugate may comprise: a targeting moiety comprising an antibody heavy chain comprising SEQ ID NO: 112 and an antibody light chain comprising SEQ ID NO: 113, a CpG ODN comprising the nucleic acid sequence of SEQ ID NO: 157, and a cleavage site comprising the amino acid sequence of SEQ ID NO: 147, wherein the cleavage site is disposed between the antibody heavy chain and the CpG ODN.

In some embodiments, there is provided a targeting conjugate comprising: (A) a first polypeptide comprising from the N-terminus to the C-terminus: a first antibody heavy chain, a first protease cleavage site (e.g., a uPA cleavage site), a first conjugation site, and a first antigen-binding fragment (e.g., an scFv, scFab or nanobody); (B) a second polypeptide comprising from the N-terminus to the C-terminus: a second antibody heavy chain, a second protease cleavage site (e.g., a uPA cleavage site), a second conjugation site, and second antigen-binding fragment (e.g., an scFv, scFab or nanobody); (C) a first antibody light chain; (D) a second antibody light chain, (E) a first oligonucleotide (e.g., a CpG oligonucleotide) comprising a first linker and a third cleavage site; (F) a second oligonucleotide (e.g., a CpG oligonucleotide) comprising a second linker and a fourth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide and the first antigen-binding fragment are released from the targeting conjugate; wherein upon cleavage of the second protease cleavage site, the second oligonucleotide and the second antigen-binding fragment are released from the targeting conjugate; wherein upon cleavage of the third cleavage site, the first oligonucleotide is released from the targeting conjugate; and wherein upon cleavage of the fourth cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 6B. An exemplary targeting conjugate may comprise: a targeting moiety comprising an antibody heavy chain comprising SEQ ID NO: 124 and an antibody light chain comprising SEQ ID NO: 125, a CpG ODN comprising the nucleic acid sequence of SEQ ID NO: 157, and a cleavage site comprising the amino acid sequence of SEQ ID NO: 147, wherein the cleavage site is disposed between the antibody heavy chain and the CpG ODN.

In some embodiments, there is provided a targeting conjugate comprising: (A) a first polypeptide comprising from the N-terminus to the C-terminus: a first antibody heavy chain, a first protease cleavage site, a first conjugation site, a second protease cleavage site, and a first antigen-binding fragment (e.g., an scFv, scFab, or nanobody); (B) a second polypeptide comprising from the N-terminus to the C-terminus: a second antibody heavy chain, a third protease cleavage site, a second conjugation site, a fourth protease cleavage site, and a second antigen-binding fragment (e.g., an scFv, scFab, or nanobody); (C) a first antibody light chain; (D) a second antibody light chain, (E) a first oligonucleotide (e.g., a CpG oligonucleotide) comprising a first linker and a fifth cleavage site; (F) a second oligonucleotide (e.g., a CpG oligonucleotide) comprising a second linker and a sixth cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first oligonucleotide and the first antigen-binding fragment are released from the targeting conjugate; wherein upon cleavage of the second protease cleavage site, the first antigen-binding fragment is released from the targeting conjugate; wherein upon cleavage of the third protease cleavage site, the second oligonucleotide and the second antigen-binding fragment are released from the targeting conjugate; wherein upon cleavage of the fourth protease cleavage site, the second antigen-binding fragment is released from the targeting conjugate; wherein upon cleavage of the fifth cleavage site, the first oligonucleotide is released from the targeting conjugate; and wherein upon cleavage of the sixth cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 6C.

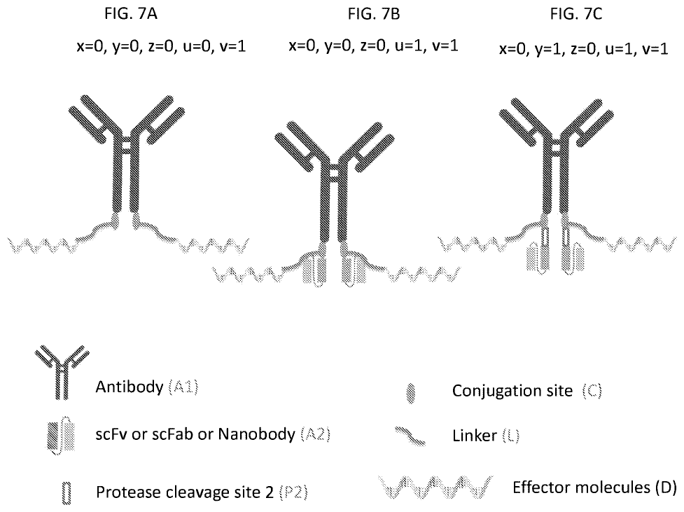

In some embodiments, there is provided a targeting conjugate comprising: (A) a first polypeptide comprising from the N-terminus to the C-terminus: a first antibody heavy chain and a first conjugation site; (B) a second polypeptide comprising from the N-terminus to the C-terminus: a second antibody heavy chain and a second conjugation site; (C) a first antibody light chain; (D) a second antibody light chain; (E) a first oligonucleotide (e.g., a CpG oligonucleotide) comprising a first linker; (F) a second oligonucleotide (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is conjugated to the first conjugation site via the first linker; wherein the second oligonucleotide is conjugated to the second conjugation site via the second linker. In some embodiments, two or more oligonucleotides are conjugated to the first conjugation site. In some embodiments, two or more oligonucleotides are conjugated to the second conjugation site. An exemplary targeting conjugate is shown in FIG. 7A. An exemplary targeting conjugate may comprise: a targeting moiety comprising an antibody heavy chain comprising SEQ ID NO: 104 and an antibody light chain comprising SEQ ID NO: 105; and a CpG ODN comprising the nucleic acid sequence selected from the group of SEQ ID NOs: 68-73.

In some embodiments, there is provided a targeting conjugate comprising: (A) a first polypeptide comprising from the N-terminus to the C-terminus: a first antibody heavy chain and a first antigen-binding fragment (e.g., an scFv/, scFab, or nanobody); (B) a second polypeptide comprising from the N-terminus to the C-terminus: a second antibody heavy chain and a second antigen-binding fragment (e.g., an scFv, scFab, or nanobody); (C) a first antibody light chain; (D) a second antibody light chain, (E) a first oligonucleotide (e.g., a CpG oligonucleotide) comprising a first linker; (F) a second oligonucleotide (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker. In some embodiments, two or more oligonucleotides are conjugated to the first conjugation site. In some embodiments, two or more oligonucleotides are conjugated to the second conjugation site. An exemplary targeting conjugate is shown in FIG. 7B. An exemplary targeting conjugate may comprise: a targeting moiety comprising an antibody heavy chain comprising SEQ ID NO: 114 or 122 and an antibody light chain comprising SEQ ID NO: 115 or 123 and a CpG ODN comprising the nucleic acid sequence selected from the group consisting of SEQ ID NOs: 68-73.

In some embodiments, there is provided a targeting conjugate comprising: (A) a first polypeptide comprising from the N-terminus to the C-terminus: a first antibody heavy chain, a first conjugation site, a first protease cleavage site, and a first antigen-binding fragment (e.g., an scFv, scFab, or nanobody); (B) a second polypeptide comprising from the N-terminus to the C-terminus: a second antibody heavy chain, a second conjugation site, a second protease cleavage site, and a second antigen-binding fragment (e.g., an scFv, scFab, or nanobody); (C) a first antibody light chain; (D) a second antibody light chain, (E) a first oligonucleotide (e.g., a CpG oligonucleotide) comprising a first linker; (F) a second effector molecule (e.g., a CpG oligonucleotide) comprising a second linker; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first antigen-binding fragment is released from the targeting conjugate; and wherein upon cleavage of the second protease cleavage site, the second antigen-binding fragment is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 7C.

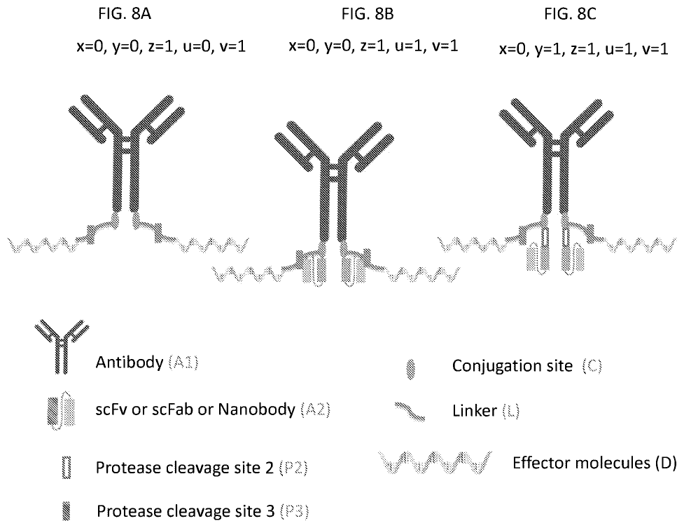

In some embodiments, there is provided a targeting conjugate comprising: (A) a first polypeptide comprising from the N-terminus to the C-terminus: a first antibody heavy chain and a first conjugation site; (B) a second polypeptide comprising from the N-terminus to the C-terminus: a second antibody heavy chain and a second conjugation site; (C) a first antibody light chain; (D) a second antibody light chain; (E) a first oligonucleotide (e.g., a CpG oligonucleotide) comprising a first linker and a first cleavage site; (F) a second oligonucleotide (e.g., a CpG oligonucleotide) comprising a second linker and a second cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is conjugated to the first conjugation site via the first linker; wherein the second oligonucleotide is conjugated to the second conjugation site via the second linker; wherein upon cleavage of the first cleavage site, the first oligonucleotide is released from the targeting conjugate; and wherein upon cleavage of the second cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 8A. An exemplary targeting conjugate may comprise: a targeting moiety comprising an antibody heavy chain comprising SEQ ID NO: 104 and an antibody light chain comprising SEQ ID NO: 105, a CpG ODN comprising the nucleic acid sequence of SEQ ID NO: 157, and a cleavage site comprising the amino acid sequence of SEQ ID NO: 147, wherein the cleavage site is disposed between the antibody heavy chain and the CpG ODN.

In some embodiments, there is provided a targeting conjugate comprising: (A) a first polypeptide comprising from the N-terminus to the C-terminus: a first antibody heavy chain and a first antigen-binding fragment (e.g., scFv/scFab/nanobody); (B) a second polypeptide comprising from the N-terminus to the C-terminus: a second antibody heavy chain and a second antigen-binding fragment (e.g., scFv/scFab/nanobody); (C) a first antibody light chain; (D) a second antibody light chain, (E) a first oligonucleotide (e.g., a CpG oligonucleotide) comprising a first linker and a first cleavage site; (F) a second oligonucleotide (e.g., a CpG oligonucleotide) comprising a second linker and a second cleavage site; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first cleavage site, the first oligonucleotide is released from the targeting conjugate; and wherein upon cleavage of the second cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 8B. An exemplary targeting conjugate may comprise: a targeting moiety comprising an antibody heavy chain comprising SEQ ID NO: 114 or 122 and an antibody light chain comprising SEQ ID NO: 115 or 123, a CpG ODN comprising the nucleic acid sequence of SEQ ID NO: 157, and a cleavage site comprising the amino acid sequence of SEQ ID NO: 147, wherein the cleavage site is disposed between the antibody heavy chain and the CpG ODN.

In some embodiments, there is provided a targeting conjugate comprising: (A) a first polypeptide comprising from the N-terminus to the C-terminus: a first antibody heavy chain, a first conjugation site, a first protease cleavage site and a first antigen-binding fragment (e.g., an scFv, scFab, or nanobody); (B) a second polypeptide comprising from the N-terminus to the C-terminus: a second antibody heavy chain, a second conjugation site, a second protease cleavage site, and a second antigen-binding fragment (e.g., an scFv, scFab, or nanobody); (C) a first antibody light chain; (D) a second antibody light chain, (E) a first oligonucleotide (e.g., a CpG oligonucleotide) comprising a first linker and a third cleavage; (F) a second effector molecule (e.g., a CpG oligonucleotide) comprising a second linker and a fourth cleavage; wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is conjugated to the first conjugation site via the first linker; wherein the second oligonucleotide is conjugated to the second conjugation site via the second linker; wherein upon cleavage of the first protease cleavage site, the first antigen-binding fragment is released from the targeting conjugate; and wherein upon cleavage of the second protease cleavage site, the second antigen-binding fragment is released from the targeting conjugate; wherein upon cleavage of the third cleavage site, the first oligonucleotide is released from the targeting conjugate; and wherein upon cleavage of the fourth cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 8C.

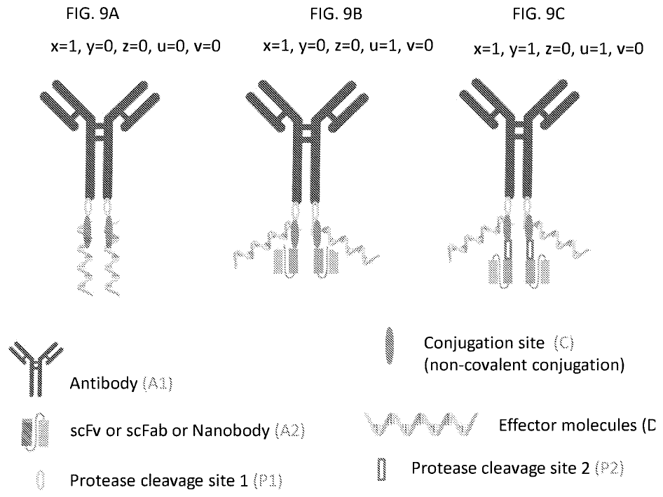

In some embodiments, there is provided a targeting conjugate comprising: (A) a first polypeptide comprising from the N-terminus to the C-terminus: a first antibody heavy chain, a first protease cleavage site, and a first non-covalent conjugation site (e.g., an oligonucleotide binding polypeptide); (B) a second polypeptide comprising from the N-terminus to the C-terminus: a second antibody heavy chain, a second protease cleavage site, and a second non-covalent conjugation site (e.g., an oligonucleotide binding polypeptide); (C) a first antibody light chain; (D) a second antibody light chain; (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is non-covalently conjugated to the first conjugation site; wherein the second oligonucleotide is non-covalently conjugated to the second conjugation site; wherein upon cleavage of the first protease cleavage site, the first oligonucleotide is released from the targeting conjugate; and wherein upon cleavage of the second protease cleavage site, the second oligonucleotide is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 9A. An exemplary targeting conjugate may comprise: a targeting moiety comprising an antibody heavy chain comprising a sequence selected from the group consisting of SEQ ID NOs: 106, 108, and 110 and an antibody light chain comprising a sequence selected from the group consisting of SEQ ID NOs: 107, 109 and 111, and a CpG ODN comprising the nucleic acid sequence of SEQ ID NO: 67.

In some embodiments, there is provided a targeting conjugate comprising: (A) a first polypeptide comprising from the N-terminus to the C-terminus: a first antibody heavy chain, a first protease cleavage site, a first non-covalent conjugation site (e.g., oligonucleotide binding polypeptide), and a first antigen-binding fragment (e.g., scFv/scFab/nanobody); (B) a second polypeptide comprising from the N-terminus to the C-terminus: a second antibody heavy chain, a second protease cleavage site, a second non-covalent conjugation site (e.g., oligonucleotide binding polypeptide), and second antigen-binding fragment (e.g., scFv/scFab/nanobody); (C) a first antibody light chain; (D) a second antibody light chain, (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is non-covalently conjugated to the first conjugation site; wherein the second oligonucleotide is non-covalently conjugated to the second conjugation site; wherein upon cleavage of the first protease cleavage site, the first oligonucleotide and the first antigen-binding fragment are released from the targeting conjugate; and wherein upon cleavage of the second protease cleavage site, the second oligonucleotide and the second antigen-binding fragment are released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 9B.

In some embodiments, there is provided a targeting conjugate comprising: (A) a first polypeptide comprising from the N-terminus to the C-terminus: a first antibody heavy chain, a first protease cleavage site, a first non-covalent conjugation site (e.g., oligonucleotide binding polypeptide), a second protease cleavage site, and a first antigen-binding fragment (e.g., scFv/scFab/nanobody); (B) a second polypeptide comprising from the N-terminus to the C-terminus: a second antibody heavy chain, a third protease cleavage site, a second non-covalent conjugation site (e.g., oligonucleotide binding polypeptide), a fourth protease cleavage site, and a second antigen-binding fragment (e.g., scFv/scFab/nanobody); (C) a first antibody light chain; (D) a second antibody light chain, (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is non-covalently conjugated to the first conjugation site; wherein the second oligonucleotide is non-covalently conjugated to the second conjugation site; wherein upon cleavage of the first protease cleavage site, the first oligonucleotide and the first antigen-binding fragment are released from the targeting conjugate; wherein upon cleavage of the second protease cleavage site, the first antigen-binding fragment is released from the targeting conjugate; wherein upon cleavage of the third protease cleavage site, the second oligonucleotide and the second antigen-binding fragment are released from the targeting conjugate; and wherein upon cleavage of the fourth protease cleavage site, the second antigen-binding fragment is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 9C.

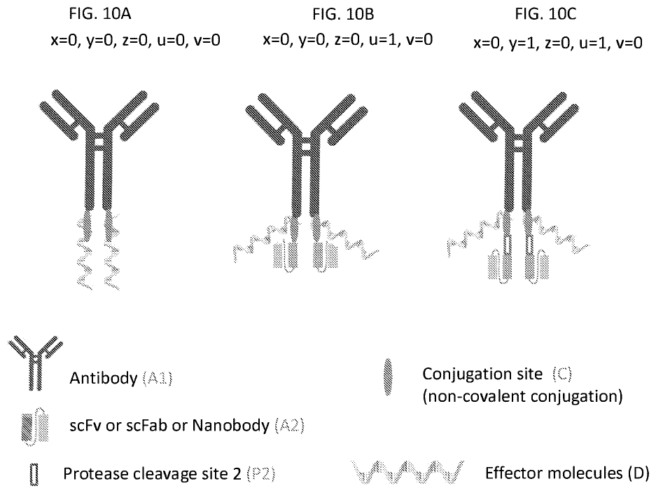

In some embodiments, there is provided a targeting conjugate comprising: (A) a first polypeptide comprising from the N-terminus to the C-terminus: a first antibody heavy chain, and a first non-covalent conjugation site (e.g., an oligonucleotide binding polypeptide); (B) a second polypeptide comprising from the N-terminus to the C-terminus: a second antibody heavy chain, and a second non-covalent conjugation site (e.g., an oligonucleotide binding polypeptide; (C) a first antibody light chain; (D) a second antibody light chain; (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first oligonucleotide is non-covalently conjugated to the first conjugation site; wherein the second oligonucleotide is non-covalently conjugated to the second conjugation site. In some embodiments, two or more oligonucleotides are conjugated to the first conjugation site. In some embodiments, two or more oligonucleotides are conjugated to the second conjugation site. An exemplary targeting conjugate is shown in FIG. 10A. An exemplary targeting conjugate may comprise: a targeting moiety comprising an antibody heavy chain comprising SEQ ID NO: 100 and an antibody light chain comprising SEQ ID NO: 101, and a CpG ODN comprising the nucleic acid sequence of SEQ ID NO: 67.

In some embodiments, there is provided a targeting conjugate comprising: (A) a first polypeptide comprising from the N-terminus to the C-terminus: a first antibody heavy chain, a first non-covalent conjugation site (e.g., oligonucleotide binding polypeptide), and a first antigen-binding fragment (e.g., an scFv, scFab, or nanobody); (B) a second polypeptide comprising from the N-terminus to the C-terminus: a second antibody heavy chain, a second non-covalent conjugation site (e.g., oligonucleotide binding polypeptide), and second antigen-binding fragment (e.g., an scFv, scFab, or nanobody); (C) a first antibody light chain; (D) a second antibody light chain, (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is non-covalently conjugated to the first conjugation site; wherein the second oligonucleotide is non-covalently conjugated to the second conjugation site. In some embodiments, two or more oligonucleotides are conjugated to the first conjugation site. In some embodiments, two or more oligonucleotides are conjugated to the second conjugation site. An exemplary targeting conjugate is shown in FIG. 10B.

In some embodiments, there is provided a targeting conjugate comprising: (A) a first polypeptide comprising from the N-terminus to the C-terminus: a first antibody heavy chain, a first non-covalent conjugation site (e.g., oligonucleotide binding polypeptide), a first protease cleavage site, and a first antigen-binding fragment (e.g., an scFv, scFab, or nanobody); (B) a second polypeptide comprising from the N-terminus to the C-terminus: a second antibody heavy chain, a second non-covalent conjugation site (e.g., oligonucleotide binding polypeptide), a second protease cleavage site, and a second antigen-binding fragment (e.g., an scFv, scFab, or nanobody); (C) a first antibody light chain; (D) a second antibody light chain, (E) a first oligonucleotide (e.g., a CpG oligonucleotide); (F) a second oligonucleotide (e.g., a CpG oligonucleotide); wherein the first antibody heavy chain and the first antibody light chain form a first antigen binding site that specifically binds to a first epitope; wherein the second antibody heavy chain and the second antibody light chain form a second antigen binding site that specifically binds to a second epitope; wherein the first antigen-binding fragment specifically binds to a third epitope; wherein the second antigen-binding fragment specifically binds to a fourth epitope; wherein the first oligonucleotide is non-covalently conjugated to the first conjugation site; wherein the second oligonucleotide is non-covalently conjugated to the second conjugation site; wherein upon cleavage of the first protease cleavage site, the first antigen-binding fragment is released from the targeting conjugate; and wherein upon cleavage of the second protease cleavage site, the second antigen-binding fragment is released from the targeting conjugate. In some embodiments, two or more oligonucleotides are conjugated to the first conjugation site. In some embodiments, two or more oligonucleotides are conjugated to the second conjugation site. An exemplary targeting conjugate is shown in FIG. 10C.

In some embodiments according to any one of the targeting conjugates described in this section, the first and the second effector molecules are the same. In some embodiments, the first and the second effector molecules are different. In some embodiments, the targeting moiety is monospecific. In some embodiments, the targeting moiety is multispecific, such as bispecific, or trispecific.

In some embodiments, there is provided a targeting conjugate comprising: (a) a first polypeptide chain and a second polypeptide chain comprising from the N-terminus to the C-terminus: an antibody heavy chain and a transglutaminase conjugation site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 100; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, ₉₄%, 95%, 96%, 97%, or 99%; or 100%) sequence identity to SEQ ID NO: 101; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain and a transglutaminase conjugation site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 104; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 105; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a transglutaminase conjugation site, a protease cleavage site, and an oligonucleotide binding site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 106; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 107; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a transglutaminase conjugation site, a protease cleavage site, and an oligonucleotide binding site, wherein the first polypeptide chain and the second polypeptide chain comprises an amino acid sequence having at least 80% (e.g., at least about any one of 85%, 90%, 91%, 92%, 93%, 94%, 95%, 96%, 97%, or 99%; or 100%) sequence identity to SEQ ID NO: 108; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 109; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a transglutaminase conjugation site, a protease cleavage site, and an oligonucleotide binding site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 110; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 111; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a protease cleavage site, and a transglutaminase conjugation site; wherein the first polypeptide chain and the second polypeptide chain comprises an amino acid sequence having at least 80% (e.g., at least about any one of 85%, 90%, 91%, 92%, 93%, 94%, 95%, 96%, 97%, or 99%; or 100%) sequence identity to SEQ ID NO: 112; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 113; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a transglutaminase conjugation site and an scFab, wherein the first polypeptide chain and the second polypeptide chain comprises an amino acid sequence having at least 80% (e.g., at least about any one of 85%, 90%, 91%, 92%, 93%, 94%, 95%, 96%, 97%, or 99%; or 100%) sequence identity to SEQ ID NO: 114; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 115; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a protease cleavage site, a transglutaminase conjugation site and an scFab, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 116; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 117; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a transglutaminase conjugation site and an scFv, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 122; (b) a third polypeptide chain and a fourth polypeptide chain comprising an antibody light chain, wherein the third and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 123; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain, a protease cleavage site, a transglutaminase conjugation site and an scFv, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 124; (b) a third polypeptide chain and a fourth polypeptide chain comprising an antibody light chain, wherein the third and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 125; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain and an scFv, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 126; (b) a third polypeptide chain and a fourth polypeptide chain comprising an antibody light chain, wherein the third and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 127; and (c) an effector molecule, wherein the effector molecule is conjugated to the transglutaminase conjugation site. In some embodiments, the effector molecule is a therapeutic agent, such as selected from compounds of Formulae (1)-(7). In some embodiments, the effector molecule is an oligonucleotide, such as a CpG oligonucleotide, e.g., an oligonucleotide comprising a nucleic acid sequence selected from the group consisting of SEQ ID NOs: 66-73 and 157.

In some embodiments, there is provided a targeting conjugate comprising: (a) a first polypeptide chain and a second polypeptide chain comprising from the N-terminus to the C-terminus: an antibody heavy chain and a transglutaminase conjugation site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 104; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 105; and (c) a CpG oligonucleotide of SEQ ID NO: 70. In some embodiments, the targeting conjugate further comprises one or more uPA cleavage sites comprising one or more amino acid sequences selected from SEQ ID NOs: 50-55.

In any of the embodiments described herein, there is provided a targeting conjugate comprising a targeting moiety comprising an antibody heavy chain comprising SEQ ID NO: 104 and an antibody light chain comprising of SEQ ID NO: 105; and and a CpG ODN comprising the nucleic acid sequence of SEQ ID NO: 70. In some embodiments, the targeting conjugate further comprises one or more uPA cleavage sites comprising one or more amino acid sequences selected from SEQ ID NOs: 50-55.

In some embodiments, there is provided a targeting conjugate comprising: (a) a first polypeptide chain and a second polypeptide chain comprising from the N-terminus to the C-terminus: an antibody heavy chain and a transglutaminase conjugation site, wherein the first polypeptide chain and the second polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 124; (b) a third polypeptide chain and a fourth polypeptide chain comprising an antibody light chain, wherein the third polypeptide chain and the fourth polypeptide chain comprise an amino acid sequence having at least 80% (e.g., at least about any one of 85%, 90%, 91%, 92%, 93%, 94%, 95%, 96%, 97%, or 99%; or 100%) sequence identity to SEQ ID NO: 125; and (c) a therapeutic agent of Compound of Formula (1). In some embodiments, the targeting conjugate further comprises one or more uPA cleavage sites comprising one or more amino acid sequences selected from SEQ ID NOs: 50-55.

In any of the embodiments described herein, there is provided a targeting conjugate comprising a targeting moiety comprising an antibody heavy chain comprising SEQ ID NO: 124 and an antibody light chain comprising of SEQ ID NO: 125; and a therapeutic agent of Compound of Formula (1). In some embodiments, the targeting conjugate further comprises one or more uPA cleavage sites comprising one or more amino acid sequences selected from SEQ ID NOs: 50-55.

### III. Methods of Use

The targeting conjugates described herein and compositions thereof (such as pharmaceutical composition) can be used to treat or diagnose various diseases, such as cancer, including solid tumor or hematological cancer, infection, inflammatory disease, autoimmune disease, and immunodeficiency disease.

Thus, in some embodiments, there is provided a method of treating a disease in an individual (such as a human), comprising administering to the individual an effective amount of any one of the targeting conjugates described herein, wherein the targeting conjugate comprises an effector molecule selected from the group consisting of a therapeutic agent and an oligonucleotide. In some embodiments, the disease is selected from the group consisting of tumor, infection, inflammatory disease, autoimmune disease, and immunodeficiency disease. In some embodiments, the disease is cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer.

In some embodiments, there is provided a method of treating a disease (such as cancer) in an individual, comprising administering to the individual an effective amount of a targeting conjugate comprising the structure of Formula I:

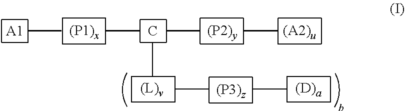

wherein: A1 is a first targeting moiety that specifically binds to a first target molecule associated with the disease; A2 is a second targeting moiety that specifically binds to a second target molecule associated with the disease; P1 is a first cleavage site; P2 is a second cleavage site; P3 is a third cleavage site; C is a conjugation site; L is a linker; D is a therapeutic agent or an oligonucleotide; x=0 or 1; y=0 or 1; z=0 or 1; u=0 or 1; v=0 or 1 a=1-20; and b=1-20. In some embodiments, the disease is selected from the group consisting of tumor, infection, inflammatory disease, autoimmune disease, and immunodeficiency disease. In some embodiments, the diseased site is a tumor site. In some embodiments, the cleavage occurs at a tumor site, for example, in a tumor microenvironment. In some embodiments, the cleavage occurs outside of a cell, such as outside of the tumor cells in a tumor microenvironment.

In some embodiments, there is provided a method of diagnosing a disease in an individual (such as a human), comprising administering to the individual an effective amount of any one of the targeting conjugates described herein, wherein the targeting conjugate comprises a detectable label, and wherein detection of the detectable label is indicative of the presence of the disease. In some embodiments, the disease is selected from the group consisting of tumor, infection, inflammatory disease, autoimmune disease, and immunodeficiency disease. In some embodiments, the disease is cancer. In some embodiments, the cancer is selected from the group consisting of bladder cancer, brain cancer, breast cancer, cervical cancer, colorectal cancer, esophageal cancer, kidney cancer, leukemia, liver cancer, lung cancer, melanoma, non-Hodgkin's lymphoma, ovarian cancer, pancreatic cancer, prostate cancer, skin cancer, thyroid cancer and uterine cancer.

In some embodiments, there is provided a method of diagnosing a disease (such as cancer) in an individual, comprising administering to the individual an effective amount of a targeting conjugate comprising the structure of Formula I:

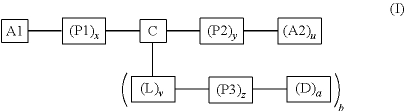

wherein: A1 is a first targeting moiety that specifically binds to a first target molecule associated with the disease; A2 is a second targeting moiety that specifically binds to a second target molecule associated with the disease; P1 is a first cleavage site; P2 is a second cleavage site; P3 is a third cleavage site; C is a conjugation site; L is a linker; D is a detectable label; x=0 or 1; y=0 or 1; z=0 or 1; u=0 or 1; v=0 or 1; a=1-20; and b=1-20; and wherein detection of the detectable label is indicative of the presence of the disease. In some embodiments, the disease is selected from the group consisting of tumor, infection, inflammatory disease, autoimmune disease, and immunodeficiency disease. In some embodiments, the diseased site is a tumor site. In some embodiments, the cleavage occurs at a tumor site, for example, in a tumor microenvironment. In some embodiments, the cleavage occurs outside of a cell, such as outside of the tumor cells in a tumor microenvironment.

In some embodiments, there is provided a method of preferentially delivering an effector molecule (e.g., a therapeutic agent, an oligonucleotide and/or a detectable label) to a diseased site in an individual in need of treatment or diagnosis with the effector molecule, comprising administering to the individual an effective amount of a targeting conjugate comprising the structure of Formula I, wherein: A1 is a first targeting moiety that specifically binds to a first target molecule associated with the disease; A2 is a second targeting moiety that specifically binds to a second target molecule associated with the disease; P1 is a first cleavage site; P2 is a second cleavage site; P3 is a third cleavage site; C is a conjugation site; L is a linker; D is the effector molecule; x=0 or 1; y=0 or 1; z=0 or 1; u=0 or 1; v=0 or 1; a=1-20; and b=1-20. In some embodiments, the disease is selected from the group consisting of tumor, infection, inflammatory disease, autoimmune disease, and immunodeficiency disease.

In some embodiments, the method increases the effective concentration of the effector molecule at a diseased site in an individual in need of treatment with the effector molecule. In some embodiments, the effective concentration of the effector molecule is increased by about any of 10%, 20%, 50%, 1×, 2×, 5×, 10× or more at the diseased site compared to administration of the effector molecule to the individual at the same effective amount.

In some embodiments, the method reduces binding of the effector molecule to a target molecule in normal tissues in an individual having a disease in need of treatment with the effector molecule. In some embodiments, administration of the targeting conjugate reduces binding of the effector molecule to its target molecule on normal cells by about any of 10%, 20%, 30%, 40%, 50%, 60%, 70%, 80%, 90% or more compared to administration of the effector molecule to the individual.

In some embodiments, the method reduces toxicity (such as on-target off-tissue toxicity) of the effector molecule to an individual having a disease in need of treatment with the effector molecule. In some embodiments, administration of the targeting conjugate reduces the toxicity of the effector molecule by about any of 10%, 20%, 30%, 40%, 50%, 60%, 70%, 80%, 90% or more compared to administration of the effector molecule at the same effective amount to the individual.

Also provided are any one of the targeting conjugates described herein for use in treating or diagnosing a disease in an individual in need thereof, or use of any one of the targeting conjugates described herein in the preparation of a medicament for treating or diagnosing a disease in an individual in need thereof.

The targeting conjugates described herein can be administered to an individual via various routes, for example, with the same route of administration as the targeting moiety in the targeting conjugate. Exemplary routes of administration include, but are not limited to parenteral, intravenous, intramuscular, and subcutaneous administration.

The effective amount, suitable dose, and dosing schedule of the targeting conjugate administered to an individual can vary depending on the particular composition, the route of administration, and the particular type of disease being treated. In some embodiments, the effective amount of the targeting conjugate is based on the effective amount of the targeting moiety and/or the effector molecule (e.g., therapeutic agent, oligonucleotide, and/or detectable label).

In some embodiments, the individual is a mammal, such as human, rodents, or primates. In some embodiments, the individual is human.

In some embodiments, the targeting conjugate is used alone or in combination with other anticancer treatments such as chemotherapeutic agents, ionizing radiation, hormonal therapy, cytokines, immunotherapy, cellular therapy, vaccines, monoclonal antibodies, antiangiogenic agents, targeted therapeutics (small molecule drugs), or biological therapies. For example, chemotherapeutic agents include, but are not limited to, antitumor alkylating agents such as Mustards (mechlorethamine HCl, melphalan, chlorambucil, cyclophosphamide, ifosfamide, busulfan), Nitrosoureas (BCNU/cannustine, CCNU/lomustine, MeCCNU/semustine, fotemustine, treptozotocin), Tetrazines (dacarbazine, mitozolomide, temozolomide), Aziridines (thiotepa, mitomycin C, AZQ/diaziquone), procarbazine HCl, hexamethylmelamine, adozelesin; cisplatin and its analogues, cisplatin, carboplatin, oxaliplatin; antimetabolites, methotrexate, other antifolates, 5-fluoropyrimidines (5-fluorouracil/5-FU), cytarabine, azacitidine, gemcitabine, 6-thiopurines (6-mercaptopurine, thioguanine), hydroxyurea; topoisomerase interactive agents epipodophyllotoxins (etoposide, teniposide), camptothecin analogues (topotecan HCl, irinotecan, 9-aminocamptothecin), anthracyclines and related compounds (doxorubicin HCl, liposomal epirubicin, daunorubicin HCl, daunorubicin HCl citrate liposomal, epirubicin, idarubicin), mitoxantrone, losoxantrone, actinomycin-D, amsacrine, pyrazoloacridine; antimicotubule agents Vinca alkaloids (vindesine, vincristine, vinblastine, vinorelbine), the taxanes (paclitaxel, docetaxel), estramustine; fludarabine, 2-chlorodeoxyadenosine, 2′-deoxycoformycin, homoharringtonine, suramin, bleomycin, L-asparaginase, floxuridine, capecitabine, cladribine, leucovorin, pentostatin, retinoids (all-trans retinoic acid, 13-cis-retinoic acid, 9-cis-retinoic acid, isotretinoin, tretinoin), pamidronate, thalidomide, cyclosporine; hormonal therapies antiestrogens (tamoxifen, toremifene, medroxyprogesterone acetate, megestrol acetate), aromatase inhibitors (aminoglutethimide, letrozole/femara, anastrozole/arirnidex, exemestane/aromasin, vorozole), gonadotropin-releasing hormone analogues, antiandrogens (flutamide, casodex), fluoxymeterone, diethylstilbestrol, octreotide, leuprolide acetate, zoladex; steroidal and non-steroidal anti-inflammatory agents (dexamethasone, prednisone); Monoclonal antibodies including, but not limited to, anti-HER2/neu antibody (herceptin/trastuzumab), anti-EGFR antibody (cetuximab/erbitux, ABX-EGF/panitumumab, nimotuzurnab), anti-CD20 antibody (rituxan/rituximab, ibritumomab/Zevalin, tositumomab/Bexxar), anti-CD33 antibody (gemtuzumab/MyloTarg), alemtuzumab/Campath, bevacizumab/A vastin; and small molecule inhibitors.

### IV. Pharmaceutical Compositions, Kits and Articles of Manufacture

Further provided by the present application are compositions (e.g., pharmaceutical compositions), kits, and articles of manufacture comprising any one of the targeting conjugates disclosed herein. In some embodiments, there is provided a pharmaceutical composition comprising any one of the targeting conjugates described herein and a pharmaceutically-acceptable carrier. In some embodiments, the composition (such as pharmaceutical composition) comprises a carrier, diluent, or excipient, which may facilitate administration of the composition to an individual in need thereof. Examples of carriers, diluents, and excipients include, but are not limited to, calcium carbonate, calcium phosphate, various sugars such as lactose, or types of starch, cellulose derivatives, gelatin, vegetable oils, polyethylene glycols and physiologically compatible solvents.

In some embodiments, the pharmaceutical composition comprises a plurality of targeting conjugates. In some embodiments, the targeting conjugate molecules in the pharmaceutical composition do not have the same number of effector molecules. In some embodiments, at least two of the targeting conjugates in the pharmaceutical composition comprise different numbers of effector molecules. The ratio between the effector molecule and the targeting moiety in the pharmaceutical compositions is within a certain range in order for the pharmaceutical composition to receive regulatory approval.

In some embodiments, the average drug loading of the pharmaceutical composition is at least about any one of 2:1, 3:1, 4:1, 5:1, 6:1, 7:1, 8:1, 9:1, 10:1, 11:1, 12:1, 13:1, 14:1, 15:1, 16:1, 17:1, 17:1, 18:1, 19:1, 20:1, 25:1, 30:1, 35:1, 40:1, 45:1, 50:1, 60:1, 70:1, 80:1, 90:1, 100:1 or more. In some embodiments, the average drug loading of the pharmaceutical composition is no more than about any one of 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, or 2:1. In some embodiments, the average drug loading of the pharmaceutical composition is about any one of 2:1-4:1, 2:1-8:1, 2:1-10:1, 2:1-16:1, 4:1-20:1, 10:1-20:1, 20:1-40:1, 40:1-100:1, 2:1-20:1, 2:1-40:1, or 10:1-40:1.

In some embodiments, the pharmaceutical composition has an average ratio of the effector molecule to the targeting moiety (e.g., the first targeting moiety and/or the second targeting moiety) of at least about any one of 1:1, 2:1, 3:1, 4:1, 5:1, 6:1, 7:1, 8:1, 9:1, 10:1, 11:1, 12:1, 13:1, 14:1, 15:1, 16:1, 17:1, 17:1, 18:1, 19:1, or 20:1. In some embodiments, the pharmaceutical composition has an average ratio of the effector molecule to the targeting moiety (e.g., the first targeting moiety and/or the second targeting moiety) of no more than about any one of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments the pharmaceutical composition has an average ratio of the effector molecule to the targeting moiety (e.g., the first targeting moiety and/or the second targeting moiety) of about any one of 1:1-2:1, 2:1-4:1, 4:1-8:1, 1:1-10:1, 1:1-16:1, 4:1-20:1, 10:1-20:1, 1:1-20:1, or 2:1-20:1.

The pharmaceutical compositions described herein can be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Other pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

In some embodiments, the pharmaceutical composition is a liquid suspension. In some embodiments, the pharmaceutical composition is a sterile composition.

Also provided are kits comprising any one of the targeting conjugates described herein. The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating or diagnosing a disease as described herein, and may have a sterile access port. The label or package insert indicates that the composition is used for treating or diagnosing a disease in an individual. The label or package insert will further comprise instructions for administering the composition to the individual.

Package insert refers to instructions customarily included in commercial packages of therapeutic products that contain information about the indications, usage, dosage, administration, contraindications and/or warnings concerning the use of such therapeutic or diagnostic products. In some embodiments, the package insert indicates that the composition is used for treating or diagnosing a disease (such as cancer).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The examples below are intended to be purely exemplary of the invention and should therefore not be considered to limit the invention in any way. The following examples and detailed description are offered by way of illustration and not by way of limitation.

### Example 1 Design and Screening of uPA Substrate Peptides

### 1. Peptide Cleavage Efficiency

Urokinase-type Plasminogen Activator (uPA) substrate peptides (SEQ ID NOs: 50-54) were screened for the efficiency of substrates cleavage by uPA but resistance to Tissue-type Plasminogen Activator (tPA) digestion. Substrate peptides and PBS were added to 384 plates and mixed with uPA or tPA. Efficiency of digestion was measure by Fluorescence Resonance Energy Transfer (FRET) method. All peptide solutions were diluted to 50 μL with 20 mM PBS pH=7.4. Experimental designs were shown in Table 2. Results are shown in Tables 3-5.

TABLE 2
Experimental Design

| | | m_{enzyme} | C_{peptide} | C_{enzyme} |
|---|---|---|---|---|
| Entry | m_{peptide}(μg) | (μg) | (mg/mL) | (mg/mL) |
| 1 | SEQ ID NO 50: 25 | uPA: 0.375 | 0.5 | 7.5 × 10⁻³ |
| 2 | SEQ ID NO 51: 25 | uPA: 0.375 | 0.5 | 7.5 × 10⁻³ |
| 3 | SEQ ID NO 52: 25 | uPA: 0.375 | 0.5 | 7.5 × 10⁻³ |
| 4 | SEQ ID NO 53: 25 | uPA: 0.375 | 0.5 | 7.5 × 10⁻³ |
| 5 | SEQ ID NO 54: 25 | uPA: 0.375 | 0.5 | 7.5 × 10⁻³ |
| 6 | SEQ ID NO 50: 25 | tPA: 0.375 | 0.5 | 7.5 × 10⁻³ |
| 7 | SEQ ID NO 51: 25 | tPA: 0.375 | 0.5 | 7.5 × 10⁻³ |
| 8 | SEQ ID NO 52: 25 | tPA: 0.375 | 0.5 | 7.5 × 10⁻³ |
| 9 | SEQ ID NO 53: 25 | tPA: 0.375 | 0.5 | 7.5 × 10⁻³ |
| 10 | SEQ ID NO 54: 25 | tPA: 0.375 | 0.5 | 7.5 × 10⁻³ |
| 11 | SEQ ID NO 50: 25 | tPA: 1.125 | 0.5 | 2.25 × 10⁻² |
| 12 | SEQ ID NO 51: 25 | tPA: 1.125 | 0.5 | 2.25 × 10⁻² |
| 13 | SEQ ID NO 52: 25 | tPA: 1.125 | 0.5 | 2.25 × 10⁻² |
| 14 | SEQ ID NO 53: 25 | tPA: 1.125 | 0.5 | 2.25 × 10⁻² |
| 15 | SEQ ID NO 54: 25 | tPA: 1.125 | 0.5 | 2.25 × 10⁻² |
| 16 | SEQ ID NO 50: 25 | 0 | 0.5 | 0 |
| 17 | SEQ ID NO 51: 25 | 0 | 0.5 | 0 |
| 18 | SEQ ID NO 52: 25 | 0 | 0.5 | 0 |
| 19 | SEQ ID NO 53: 25 | 0 | 0.5 | 0 |
| 20 | SEQ ID NO 54: 25 | 0 | 0.5 | 0 |

uPA effectively cleaved peptides of SEQ ID NOs: 50-54, as shown in Table 3 below:

TABLE 3
uPA cleavage for SEQ ID NOs: 50-54

| Fluorescence | SEQ ID | SEQ ID | SEQ ID | SEQ ID | SEQ ID |
|---|---|---|---|---|---|
| Intensity | NO: 50 | NO: 51 | NO: 52 | NO: 53 | NO: 54 |
| Time to Reach | 1.7 | 1.8 | 1.4 | 1.6 | 1.4 |
| Plateau (h) | | | | | |
| Fold | 4.9 | 9.6 | 3.1 | 3.0 | 4.7 |
| of Increase | | | | | |

The shorter the time it took to reach plateau in fluorescence intensity, the faster the uPA digestion. The higher the fold of increase in fluorescence intensity, the more efficient the uPA digestion. Therefore, the rankings of uPA digestion rates for the peptides were: SEQ ID NOs: 52, 54>SEQ ID NO: 53>SEQ ID NO: 50>SEQ ID NO: 51. The rankings of reaction efficiency were: SEQ ID NO: 51>SEQ ID NO: 50>SEQ ID NO: 54>SEQ ID NO: 52>SEQ ID NO: 53.

At 7.5×10⁻³ mg/mL tPA concentration, only SEQ ID NOs: 51 and 52 were mildly digested, as shown in Table 4:

TABLE 4
tPA digestion at 7.5 × 10⁻³ mg/mL concentration

| Fluorescence | SEQ ID | SEQ ID | SEQ ID | SEQ ID | SEQ ID |
|---|---|---|---|---|---|
| Intensity | NO: 50 | NO: 51 | NO: 52 | NO: 53 | NO: 54 |
| Fold of Increase | 0.63 | 2.1 | 1.2 | 0.73 | 0.91 |

At 2.25×10⁻² mg/mL tPA concentration, SEQ ID NOs: 51, 52 and 54 were digested, as shown in Table 5:

TABLE 5
tPA digestion at 2.25 × 10⁻² mg/mL concentration

| Fluorescence | SEQ ID No | SEQ ID No | SEQ ID No | SEQ ID No | SEQ ID No |
|---|---|---|---|---|---|
| Intensity | 50 | 51 | 52 | 53 | 54 |
| Fold of Increase | 1.1 | 5.4 | 2.1 | 1.3 | 2.6 |

### 2. Stability of the Substrate Peptides in Plasma Measured by FRET

Plasma was mixed with antibiotics (penicillin-streptomycin) and then peptides of SEQ ID NOs: 50-54 were added to the plasma mixture. The reaction mixture was incubated at room temperature. Samples were taken at 0 hour, 8 hours, 24 hours, 32 hours, 48 hours, 72 hours, 96 hours, 120 hours and 144 hours for FRET measurements. The experimental design is shown in Table 6.

TABLE 6
Experimental Design

| | V_{peptide} | V_{PS} | Vₚₗₐₛₘₐ | Vₜₒₜₐₗ | C_{peptide} |
|---|---|---|---|---|---|
| No. | (μL) | (μL) | (mL) | (mL) | (mM) |
| 1 | SEQ ID NO: 50 | 62.5 | 1.0289 | 1.25 | 0.5 |
| | 158.6 | | | | |
| 2 | SEQ ID NO: 51 | 62.5 | 1.0799 | 1.25 | 0.5 |
| | 107.6 | | | | |
| 3 | SEQ ID NO: 52 | 62.5 | 0.9839 | 1.25 | 0.5 |
| | 203.6 | | | | |
| 4 | SEQ ID NO: 53 | 62.5 | 0.9699 | 1.25 | 0.5 |
| | 217.6 | | | | |
| 5 | SEQ ID NO: 54 | 62.5 | 0.9433 | 1.25 | 0.5 |
| | 244.2 | | | | |
| 6 | 0 | 62.5 | 1.1875 | 1.25 | 0 |

Fold of increase in fluorescence intensity for each of the substrate peptides in plasma is shown in Table 7 below:

TABLE 7
Fold increase of fluorescence intensity for SEQ ID NOs: 50-54

| Fluorescence | SEQ ID | SEQ ID | SEQ ID | SEQ ID | SEQ ID |
|---|---|---|---|---|---|
| Intensity | NO: 50 | NO: 51 | NO: 52 | NO: 53 | NO: 54 |
| Fold of Increase | 10.4 | 6.8 | 7.3 | 5.4 | 16.8 |

The rankings of peptides stability in the plasma are approximately: SEQ ID NO: 53>SEQ TD NOs: 51, 52>SEQ ID NO: 50>SEQ ID NO: 54.

3. Stability of the Substrate Peptides in Plasma from Kinetic Measurements

Experiments were designed as shown in Table 8. Plasma-peptide mixtures were incubated at room temperature for 12 hours and fluorescence intensities were measured continuously. Results are shown in Table 9. PS: penicillin-streptomycin.

TABLE 8
Experimental Design

| | V_{peptide} | V_{PS} | Vₚₗₐₛₘₐ | Vₜₒₜₐₗ | C_{peptide} |
|---|---|---|---|---|---|
| No. | (μL) | (μL) | (mL) | (mL) | (mM) |
| 1 | SEQ ID NO 50: 6.34 | 2.5 | 35.86 | 50 | 0.5 |
| 2 | SEQ ID NO 51: 4.30 | 2.5 | 37.9 | 50 | 0.5 |
| 3 | SEQ ID NO 52: 8.14 | 2.5 | 39.36 | 50 | 0.5 |
| 4 | SEQ ID NO 53: 8.70 | 2.5 | 37 | 50 | 0.5 |
| 5 | SEQ ID NO 54: 9.77 | 2.5 | 37.08 | 50 | 0.5 |
| 6 | 0 | 2.5 | 42.2 | 50 | 0 |

TABLE 9
Kinetic measurements of substrate stability

| Fluorescence | SEQ ID | SEQ ID | SEQ ID | SEQ ID | SEQ ID |
|---|---|---|---|---|---|
| Intensity | NO: 50 | NO: 51 | NO: 52 | NO: 53 | NO: 54 |
| Time to Reach | 4.8 | 3.6 | No Plateau | No Plateau | 3.6 |
| Plateau (h) | | | in 12 h | in 12 h | |
| Fold of Increase | 2.8 | 2.3 | 2.0 | 1.7 | 2.7 |

The rankings of peptide stability in the plasma were approximately SEQ ID NOs: 52, 53>SEQ ID NO: 50>SEQ ID Nos: 51, 54. For SEQ ID NOs 52 and 53, the fluorescence intensities increased with time to 2.0-fold and 1.7-fold respectively, but did not reach a plateau within 12 hours. SEQ ID NO: 50 reached plateau in 4.8 hours, and the maximum fold of increase in fluorescence intensity was 2.8-fold. The fluorescence intensities of SEQ ID NOs: 51 and 54 increased to 2.3-fold and 2.7-fold within 3.6 hours, respectively.

### 4. uPA Digestion in Antibodies

Antibodies Ab3C (heavy chain sequence SEQ ID NO: 108, light chain sequence SEQ ID NO: 109), Ab3E (heavy chain sequence SEQ ID NO: 110, light chain sequence SEQ ID NO: 111) and Ab5D (heavy chain sequence SEQ ID NO: 124, light chain sequence SEQ ID NO:125) containing a uPA substrate sequence in the heavy chain were used to test uPA digestion efficiency in antibodies. Antibody Ab5C (heavy chain sequence SEQ ID NO: 122, light chain sequence SEQ ID NO:123) that does not contain a uPA substrate sequence was used as a control. Experimental design is shown in Table 10 below. The reaction mixtures were incubated at 37° C. for 24 hours.

TABLE 10
Experimental design

| | | | | | 50 mM | | |
|---|---|---|---|---|---|---|---|
| | Antibody¹ | V_{uPA} | C_{antibody} | C_{uPA}² | EDTA³ | V_{ddH2O} | Vₜₒₜₐₗ |
| NO. | (μL) | (μL) | (mg/mL) | (mg/mL) | (μL) | (μL) | (μL) |
| 1 | Ab3C | 15 | 0.7 | 0.05 | 1.5 | 33.5 | 75 |
| | 25.0 | | | | | | |
| 2 | Ab3E | 15 | 0.8 | 0.05 | 1.5 | 2.4 | 75 |
| | 56.1 | | | | | | |
| 3 | Ab5C: | 6.4 | 1 | 0.02 | / | 62.94 | 80 |
| | 8.7 | | | | | | |
| 3 | Ab5D: | 6.4 | 1 | 0.02 | / | 64.9 | 80 |
| | 10.26 | | | | | | |

¹Initial concentrations of Ab3C, Ab3E, Ab5C, and Ab5D were 2.12 mg/mL, 1.07 mg/mL, 9.2 mg/mL, and 7.8 mg/mL, respectively.
²Initial concentration of uPA was 0.25 mg/mL.
³Concentration of EDTA was 1 mM.

uPA digestion products were analyzed with LC-MS. The heavy chains of Ab3C, Ab3E and Ab5D were cleaved by uPA to yield fragments of expected molecular weight. Ab5C was not cleaved by uPA.

### Example 2. Design, Synthesis and Analysis of the Drug-Linkers

### 1. Purpose of the Experiments

(1) Design MMAE drug-linkers containing PEG spacers of different molecular weight, investigate coupling efficiency of TGase enzyme catalyzed conjugation of MMAE drug-linkers to polypeptides and antibodies, and screen for PEG spacers and drug-linkers with high coupling efficiency. (2) Design linkers to connect to different locations of SN-38, and screen for SN-38 drug-linkers with high coupling efficiency, stability and high bioactivity.

### 2. Synthesis of Drug Linkers

Compounds of Formulae (1)-(7) were chemically synthesized and tested by HPLC, LC-MS, and H-NMR. The drug linkers were used in subsequent experiments after meeting the quality standards.

### 3. Restriction Enzyme Digestion of the Drug-Linkers by CathepsinB

To simulate the release of drug under physiological conditions, the drug-linkers were subjected to cleavage by CathepsinB, after which an elimination reaction occurred and unmodified drug was released.

### 3.1 Mechanism of Reaction

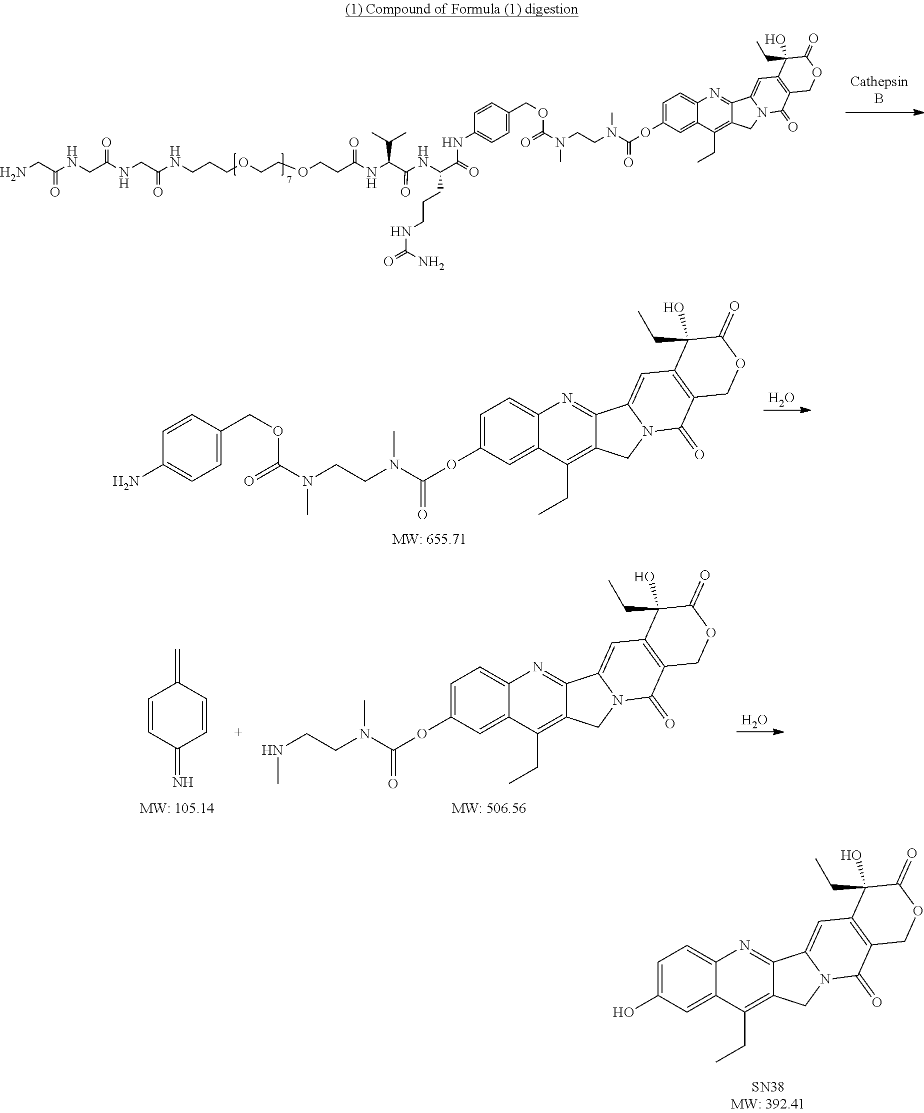

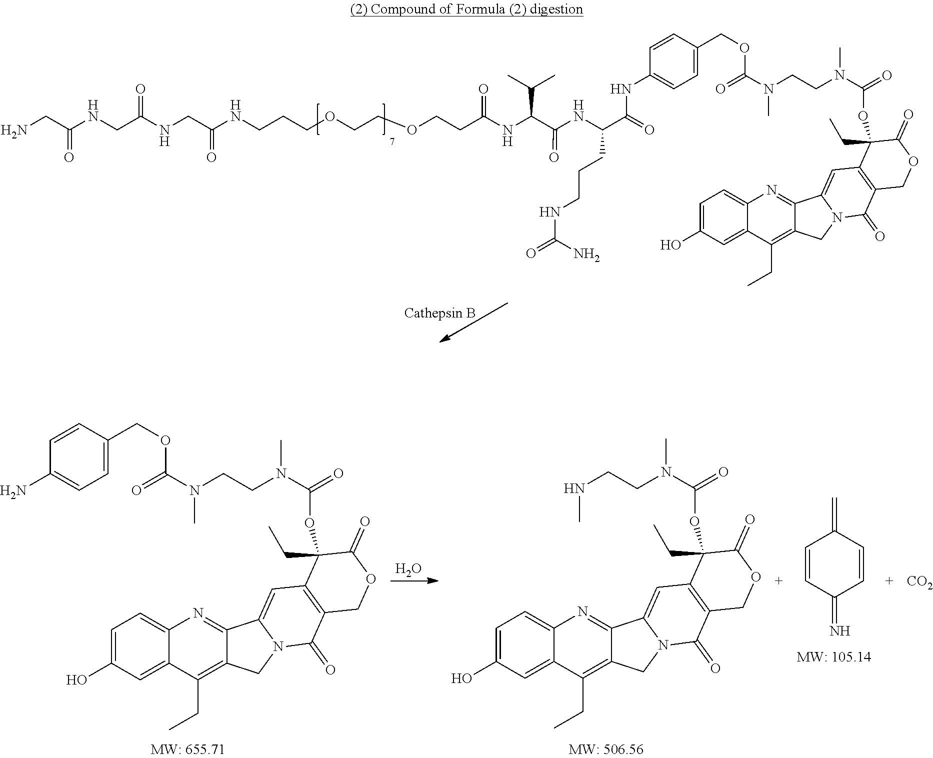

### 3.2 Experimental Method

Lyophilized CathepsinB enzyme powder was dissolved in 130 μL of pure water, and divided into 13 tubes of 10 μL each. At this time, the concentration of each tube was 0.15 mg/ml (4.15 μM). Activation buffer (20 mM sodium acetate, 30 mM DTT, 15 mM EDTA, pH 5.5) was then added to a tube of enzyme to activate the enzyme for 1 hour at room temperature. The activated enzyme was diluted to 125 nM with enzyme digestion buffer (25 mM sodium acetate, 1 mM EDTA, pH 5.5). Drug-linkers were added to the activated enzyme to a final concentration of 62.5 μM, and incubated at 37° C. for 3 hours.

### 3.3 Results and Conclusion

Compounds of Formulae (1) and (2) were both effectively digested. Compound of Formula (1) was digested to release SN38, and compound of Formula (2) was digested to release SN38 derivatives.

### 4. Stability Investigation

Stability of the drugs was tested in buffers of different pH, plasma and dynamic pH solutions by measuring lactone and products of lactone ring opening in compound of Formula (1), compound of Formula (2) and SN-38.

### 4.1 Stability of SN-38

A hydrochloric acid solution with a pH of 4 and a sodium hydroxide solution with a pH of 10 were prepared respectively. 5 mg of SN-38 was dissolved in 5 mL of acetonitrile. The stability of SN-38 in a solution of changing pH (pH=10 to pH=4) was measured by shaking SN-38 at room temperature under pH=10 for 10 min, then adjusting the pH back to 4 and shake at room temperature for 10 min before sampling. SN-38 was then incubated at room temperature overnight (16 h) on a shaker and assayed afterwards.

TABLE 11
Experimental design of SN-38 stability test

| | Final | Final | Initial | Volume of | |
|---|---|---|---|---|---|
| | conc. | volume | conc. | SN-38 | Buffer |
| pH | (mg/mL) | (μL) | mg/mL) | (μL) | (μL) |
| 4 | 0.5 | 200 | 1 | 100 | 100 |
| 10 | 0.5 | 200 | 1 | 100 | 100 |
| 10→4 | 0.5 | 200 | 1 | 100 | 100 |

### 4.2 Stability of Compounds of Formulae (1) and (2) in Buffer

Preparation of 20 mM phosphate buffer with pH=7.4 (in 1 L): 8.0 g sodium chloride, 0.2 g potassium chloride, 7.3 g disodium hydrogen phosphate dodecahydrate, and 0.48 g potassium dihydrogen phosphate was dissolved in water and dilute to 1 L at pH 7.4.

The buffer was adjusted to pH 9.0, 7.0 and 5.0 with saturated phosphoric acid solution and sodium hydroxide solution. When investigating the stability of the drug in a changing pH (9 to 5) solution, compound of Formula (1)/(2) was incubated on a shaker at room temperature at pH 9 for 10 minutes. The solution pH was adjusted back to 5, and shaken overnight at room temperature (16 h) before testing.

TABLE 12
Experimental design of compound of Formula (1)/(2)
stability test in buffer

| | Final | Final | Initial | | | |
|---|---|---|---|---|---|---|
| | conc. | volume | conc. | Volume | DMSO | Buffer |
| pH | (mg/mL) | (μL) | (mg/mL) | (μL) | (μL) | (μL) |
| 5 | 0.5 | 120 | 30 | 2 | 10 | 108 |
| 7 | 0.5 | 120 | 30 | 2 | 10 | 108 |
| 9 | 0.5 | 120 | 30 | 2 | 10 | 108 |
| 9→5 | 0.25 | 120 | 30 | 1 | 10 | 109 |

### 4.3 Stability of Compound of Formulae (1) and (2) in Plasma

Preparation of protein precipitation solution: 1 g NaCl was added to 3 mL methanol and 5 mL acetonitrile, stirred for 1 h, and then allowed to stand at room temperature for 1 h before use.

200 μL protein precipitation solution was then added to the reaction solution and centrifuged at 8000 g for 10 min. The supernatant was collected for analysis.

TABLE 13
Experimental design of compound of
Formula (1)/(2) stability test in plasma

| | Final | Total | Initial | Volume | |
|---|---|---|---|---|---|
| | concentration | volume | concentration | of drug | Buffer |
| pH | (mg/mL) | (μL) | (mg/mL) | (μL) | (μL) |
| 7.4 | 0.5 | 120 | 30 | 2 | 118 |

### 4.4 Results and Conclusion

SN-38 existed as a lactone under acidic conditions (94%), and under alkaline conditions as a lactone ring-opened form (92%). After SN-38 was subjected to alkaline conditions for 10 minutes and then subjected to acidic conditions before testing, 56% of SN-38 was present in the form of lactone, and 42% of lactone ring-opening products were present. After incubation overnight, 86% was in the form of lactone, and 13% was present in the form of lactones ring-opening products.

Compound of Formula (1) mainly existed in the form of lactones under acidic conditions (88%), and under alkaline conditions, it mainly exists as lactones in the form of ring opening (92%). After compound of Formula (1) was subjected to alkaline conditions for 10 minutes and then subjected to acidic conditions before testing, 98% was in the form of lactones, and 0.21% was in the form of lactone-opening products. Compound of Formula (1) was partially hydrolyzed in plasma, mainly in the form of lactone opening;

Compound of Formula (2) was less affected by pH with 98% existed in the form of lactones in buffer. The stability of compound of Formula (2) in the plasma was also better than that of compound of Formula (2). Compound of Formula (2) was mainly in the form of lactones in the plasma, and ring-opening products were basically invisible.

### Example 3. Design and Synthesis of CpG-ODN-Linker

CpG ODNs (Cytosine-phosphodiester-Guanine Oligodeoxynucleotides) and linkers of SEQ ID NOs: 66-73, and Formula VIII were designed, synthesized and tested for their coupling efficiency to TGase substrate sequence upon catalysis of TGase. CpG-ODNs were synthesized with phosphoramidite chemistry, and characterized by electrophoresis and mass spectroscopy. The formulas of CpG ODN linkers were 5′Amino Modifier-Spacer-Pₕ-CpG ODN (Formula IV) and 3′Amino Modifier-Spacer-Pₕ-CpG ODN (Formula V), wherein Spacer is (CH₂)ₙ-(PEG)ₘ, h, n and m are integers, h=0 or 1, n≥1, and m≥0, P is a cleavage site.

### Example 4. Design and Screening of TGase Conjugation Peptides

### 1. Purpose of Experiment

Coupling efficiency between substrate peptides (SEQ ID Nos:1-12) and small molecule toxins (drug-linker, Formulas II-V) using TGase (transglutaminase) was investigated. Substrate peptides with high coupling efficiency were selected for the development of peptide conjugates, protein conjugates and antibody conjugates.

TABLE 14
Sequences of the substrate peptides

| TGase SEQ ID NOs | Peptide sequences |
|---|---|
| SEQ ID NO 1 | LLQG LLQG LLQG |
| SEQ ID NO 2 | LLQG GSG LLQG GSG LLQG |
| SEQ ID NO 3 | LQSP LQSP LQSP |
| SEQ ID NO 4 | LQSP GSG LQSP GSG LQSP |
| SEQ ID NO 5 | PNPQLPF PNPQLPF PNPQLPF |
| SEQ ID NO 6 | PNPQLPF GSG PNPQLPF GSG PNPQLPF |
| SEQ ID NO 7 | PKPQQFM PKPQQFM PKPQQFM |
| SEQ ID NO 8 | PKPQQFM GSG PKPQQFM GSG PKPQQFM |
| SEQ ID NO 9 | GQQQLG GQQQLG GQQQLG |
| SEQ ID NO 10 | GQQQLG GSG GQQQLG GSG GQQQLG |
| SEQ ID NO 11 | RLQQP RLQQP RLQQP |
| SEQ ID NO 12 | RLQQP GSG RLQQP GSG RLQQP |

### 2. Peptide Synthesis and Analysis

The peptides were synthesized by conventional solid-phase synthesis method and analyzed by HPLC and LC-MS.

### 3. Peptide Synthesis and Analysis

Microbial/bacterial transglutaminase (MTG/BTG) was used herein. Enzyme activity of the MTG/BTG was determined by conventional methods in the art. The TGase was then used for crosslinking the drug linkers with peptides of SEQ ID NOs: 1-12. Reactivity between the peptide and the drug linker was determined by HPLC peak area of the product.

Peptides were diluted with PBS at pH=7.4 to 10 mg/mL. Drug linkers (compounds of Formulae (1)-(4)) were dissolved in DMSO to 31 mg/mL. The reaction mixture contained peptides at a final concentration of 1 mg/mL, a final concentration of DMSO of 20%, and the molar ratio of peptide:drug linker was 1:5. TGase was added to the reaction mixture to a final concentration of 25 U/mL. The reaction was measured by RP-HPLC after 16 hours at room temperature. The results are shown in Table 15.

TABLE 15
HPLC peak areas of cross-linking products. DPR: drug peptide ratio of the final
product

| Drug | Compound of | Compound of | Compound of | Compound of |
|---|---|---|---|---|
| Peptide | Formula (1) | Formula (2) | Formula (3) | Formula (4) |
| SEQ ID NO: 1 | DPR = 3: 26.0% | DPR = 3: 25% | N.D. | DPR = 3: 10% |
| | DPR = 1:6% | DPR = 2: 6% | | |
| SEQ ID NO: 2 | DPR = 3: 51.5% | DPR = 1: 5.5% | N.D. | DPR = 3: 45% |
| | DPR = 2: 3.9% | DPR = 2: trace | | DPR = 2: 2% |
| SEQ ID NO: 3 | DPR = 3: 60.0% | DPR = 1: 4.6% | DPR = 3: 7% | DPR = 3: 44% |
| | DPR = 2: 2.0% | | | DPR = 2: 2.5% |
| SEQ ID NO: 4 | DPR = 3: 61.9% | DPR = 1: 9.2% | DPR = 3: 12% | DPR = 3: 42% |
| | DPR = 2: 2.0% | | | DPR = 2: 2% |
| SEQ ID NO: 5 | DPR = 3: 61.9% | DPR = 1: 16.9% | DPR = 1:33.1% | DPR = 2: 1.7% |
| | DPR = 2: 2.0% | DPR = 2: 5.3% | | |
| SEQ ID NO: 6 | DPR = 3: 44.4% | DPR = 2: 5.2% | DPR = 1:18.1% | DPR = 2: 48.2% |
| | DPR = 2: 18.4% | DPR = 1: 4.0% | | |
| SEQ ID NO: 7 | DPR = 3: 36.1% | N.D. | DPR = 2:31% | DPR = 3: 49.7% |
| | DPR = 4: trace | | | |
| SEQ ID NO: 8 | DPR = 3: 34.5% | DPR = 1: 1.7% | DPR = 2: 41.5% | DPR = 2: 60.2% |
| | DPR = 4: trace | | | |
| SEQ ID NO: 9 | DPR = 3: 20.8% | DPR = 1: 2.6% | DPR = 1:47.7% | N.D. |
| | DPR = 2: 20% | | | |
| | DPR = 4: 9.2% | | | |
| SEQ ID | DPR = 2: 20.8% | DPR = 1: 1.5% | DPR = 1: 57.8% | DPR = 1: trace |
| NO: 10 | DPR = 1: 20.3% | | | |
| | DPR = 3: 12.6% | | | |
| | DPR = 4: 3.7% | | | |
| SEQ ID | DPR = 3: 45.5% | DPR = 1: 1.6% | DPR = 1:7.1% | DPR = 2: 57.3% |
| NO: 11 | | | | |
| SEQ ID | DPR = 3: 47% | DPR = 1: 5.4% | DPR = 1: 46.9% | N.D. |
| NO: 12 | | | | |

### 4. Conclusions

Between the SN-38 drugs, compound of Formula (1) that reacts with the linker at the hydroxyl group at position 10 had a higher reactivity compared to compound of Formula (2), which reacts with the linker at position 20. Between the two MMAE drugs, compound of Formula (4) (8 PEG spacers) had a higher reactivity compared to compound of Formula (3) (4 PEG spacers).

Compound of Formula (1) was able to crosslink to SEQ ID NOs: 7-10 with DPR=4, to SEQ ID NOs 1-6 and 11-12 with DPR=3 plus some DPR=2 products. The overall order of crosslinking reactivity of the peptides was approximately: SEQ ID NO: 9≈SEQ ID NO: 10>SEQ ID NO: 7≈SEQ ID NO: 8>SEQ ID NO: 5≈SEQ ID NO: 4 z SEQ ID NO: 3>SEQ ID NO: 2>SEQ ID NO: 6>SEQ ID NO: 12≈SEQ ID NO: 11>SEQ ID NO: 1.

Compound of Formula (2) was able to crosslink to SEQ ID NO:1 with DPR=3, to SEQ ID NOs: 2, 5, and 6 with DPR=2, and with DPR=1 to the other peptides tested. The overall order of crosslinking reactivity of the peptides was approximately: SEQ ID NO: 1>SEQ ID NO: 5>SEQ ID NO: 6>SEQ ID NO: 4>SEQ ID NO: 2>SEQ ID NO: 12>SEQ ID NO: 3>SEQ ID NO:s: 8, 9, 10, 11>SEQ ID NO: 7.

Compound of Formula (3) mostly crosslinked to peptides with DPR=1. The overall order of crosslinking reactivity of the peptide was approximately: SEQ ID NOs: 8, 9, 10, 12>SEQ ID NOs: 5, 7>SEQ ID NOs: 4, 6>SEQ ID NO: 11>SEQ ID NO: 3>SEQ ID NOs: 1, 2.

Compound of Formula (4) was able to crosslink to SEQ ID NOs: 2, 3, 4, and 7 with DPR=3, to SEQ ID NOs: 6, 8, 11 with DPR=2, and the overall order of crosslinking reactivity of the peptides was approximately: SEQ ID NOs: 2, 3, 4, 7>SEQ ID NO: 8>SEQ ID NOs: 6, 11>SEQ ID NO: 1>SEQ ID NOs: 5, 10>SEQ ID NOs: 9, 12.

### Example 5. Characterization of the Antibodies

Monospecific PD-L1 antibodies Ab2B (heavy chain SEQ ID NO:100, light chain SEQ ID NO:101), Ab3A (heavy chain SEQ ID NO:104, light chain SEQ ID NO:105), Ab3B (heavy chain SEQ ID NO:106, light chain SEQ ID NO:107), Ab3C (heavy chain SEQ ID NO:108, light chain SEQ ID NO:109), Ab3E (heavy chain SEQ ID NO:110, light chain SEQ ID NO:111), Ab3F (heavy chain SEQ ID NO:112, light chain SEQ ID NO:113), and bispecific PD-L1/Trop-2 antibodies Ab4A (heavy chain SEQ ID NO:114, light chain SEQ ID NO:115), Ab4B (heavy chain SEQ ID NO:116, light chain SEQ ID NO:117), Ab5A (heavy chain SEQ ID NO:118, light chain SEQ ID NO:119), Ab5B (heavy chain SEQ ID NO:120, light chain SEQ ID NO:121), Ab5C (heavy chain SEQ ID NO:122, light chain SEQ ID NO:123), Ab5D (heavy chain SEQ ID NO:124, light chain SEQ ID NO:125), Ab5E (heavy chain SEQ ID NO:126, light chain SEQ ID NO:127), and Ab5F (heavy chain SEQ ID NO:128, light chain SEQ ID NO:129) were expressed and tested for affinity to human PD-L1/B7-H1/CD274 and further to human Trop-2/TACSTD2 protein for the bispecific antibodies. The results of affinity to PD-L1 are shown in Table 20. The results of affinity to Trop-2 are shown in Table 21. The results show that both the monospecific and bispecific antibodies maintained high affinities to the antigen(s).

TABLE 16
Affinities of antibodies to PD-L1

| NO | Ligand | ka(1/Ms) | kd(1/s) | KD(M) |
|---|---|---|---|---|
| 1 | Ab2B | 1.125E+06 | 1.658E−04 | 1.474E−10 |
| 2 | Ab3A | 7.363E+05 | 2.124E−04 | 2.884E−10 |
| 3 | Ab3B | 1.018E+06 | 1.163E−04 | 1.142E−10 |
| 4 | Ab3C | 1.010E+06 | 2.399E−04 | 2.375E−10 |
| 5 | Ab3E | 8.390E+05 | 2.527E−04 | 3.012E−10 |
| 6 | Ab3F | 7.311E+05 | 2.225E−04 | 3.044E−10 |
| 7 | Ab4A | 9.742E+05 | 2.797E−04 | 2.871E−10 |
| 8 | Ab4B | 9.160E+05 | 2.247E−04 | 2.453E−10 |
| 9 | Ab5A | 1.091E+06 | 2.649E−04 | 2.428E−10 |
| 10 | Ab5B | 1.772E+06 | 2.716E−04 | 1.533E−10 |
| 11 | Ab5C | 1.781E+06 | 2.822E−04 | 1.572E−10 |
| 12 | Ab5D | 1.137E+06 | 2.703E−04 | 2.377E−10 |
| 13 | Ab5E | 1.611E+06 | 2.823E−04 | 1.753E−10 |
| 14 | Ab5F | 8.818E+05 | 2.205E−04 | 2.500E−10 |

TABLE 17
Affinities of antibodies to Trop-2

| NO | Ligand | ka(1/Ms) | kd(1/s) | KD(M) |
|---|---|---|---|---|
| 1 | Ab5A | 9.810E+04 | 1.777E−04 | 1.811E−09 |
| 2 | Ab5B | 1.031E+05 | 1.569E−04 | 1.521E−09 |
| 3 | Ab5C | 1.048E+05 | 1.990 E−04 | 1.899E−09 |
| 4 | Ab5D | 8.478E+05 | 2.090E−04 | 2.465E−09 |
| 5 | Ab5E | 6.938E+04 | 2.672E−04 | 3.852E−09 |
| 6 | Ab5F | 5.146E+04 | 2.793E−04 | 5.427E−09 |
| 7 | Ab4A | 1.620E+05 | 1.507E−04 | 9.302E−10 |
| 8 | Ab4B | 1.148E+05 | 1.304E−04 | 1.135E−09 |

### Example 6. Synthesis and Characterization of Antibody Drug Conjugate (ADC)

### 1. Antibody-Drug Conjugation of Monospecific Antibody

Drug-linkers (compounds of Formulae (1) and (2)) were conjugated to anti-PD-L1 antibody Ab3A of SEQ TD NOs: 104 (comprising a heavy chain and a TGase substrate sequence) and 105 (a light chain), or anti-PD-L1 antibody Ab3F of SEQ ID NOs: 112 (comprising a heavy chain, uPA substrate peptide, and TGase substrate sequence) and 113 (a light chain). Experimental design of the conjugation experiment is shown in Table 18. Reaction mixtures were incubated at room temperature for 16 hours, and drug-to-antibody ratio (DAR) of the conjugation product was determined by mass spectrometry.

TABLE 18
Experimental design

| | | | | Reaction mixture | | | | |
|---|---|---|---|---|---|---|---|---|
| | | | | | | Drug to | | |
| | | | | | | mAb | | |
| | | | | | | molar | | Tween- |
| Entry | ADC | Drug-linker | mAb | TGase | DMSO | ratio | Propanediol | 80 |
| 1 | Ab3A- | Compound | Ab3A | 5 U/mL | 15% | 40:1 | 40% | 0.2% |
| | CD-1 | of Formula | | | | | | |
| | | (1) | | | | | | |
| 2 | Ab3A- | Compound | Ab3A | 5 U/mL | 15% | 40:1 | 40% | 0.2% |
| | CD-2 | of Formula | | | | | | |
| | | (2) | | | | | | |
| 3 | Ab3F- | Compound | Ab3F | 5 U/mL | 15% | 40:1 | 40% | 0.2% |
| | CD-1 | of Formula | | | | | | |
| | | (1) | | | | | | |
| 4 | Ab3F- | Compound | Ab3F | 5 U/mL | 15% | 40:1 | 40% | 0.2% |
| | CD-2 | of Formula | | | | | | |
| | | (2) | | | | | | |

For ADC Ab3A-CD-1, the DAR value is 3.93, and for Ab3A-CD-2, the DAR value is 3.77. For ADC Ab3F-CD-1, the DAR value is 4.98, and for Ab3F-CD-2, the DAR value is 4.85.

### 2. Antibody-Drug Conjugation of Bispecific Antibodies

Drug linkers (compounds of Formulae (1) and (2)) were conjugated to anti-PD-L1 and anti-Trop-2 bispecific antibodies Ab5C (heavy chain SEQ ID NO: 122, light chain SEQ TD NO: 123) and Ab5D (heavy chain SEQ TD NO: 124, light chain SEQ ID NO: 125). Drug-to-antibody ratio (DAR) of the conjugation product was determined by mass spectrometry. Reaction mixtures were incubated at room temperature for 16 hours. Experimental design of the conjugation experiment and DAR values of the ADCs shown in Table 19.

TABLE 19
Experimental design and DAR of the ADC

| | | | | Reaction mixture | | | | | |
|---|---|---|---|---|---|---|---|---|---|
| | | | | | | Drug to | | | |
| | | | | | | mAb | | | DAR |
| | | | | | | molar | | Tween- | of |
| Entry | ADC | Drug linker | mAb | TGase | DMSO | ratio | Propanediol | 80 | ADC |
| 1 | Ab5C- | Compound | Ab5C | 5 U/mL | 15% | 40:1 | 40% | 0.2% | 4.81 |
| | CD-1 | of Formula | | | | | | | |
| | | (1) | | | | | | | |
| 2 | Ab5D- | Compound | Ab5D | 5 U/mL | 15% | 40:1 | 40% | 0.2% | 5.65 |
| | CD-1 | of Formula | | | | | | | |
| | | (1) | | | | | | | |
| 3 | Ab5C- | Compound | Ab5C | 5 U/mL | 15% | 40:1 | 40% | 0.2% | 4.64 |
| | CD-2 | of Formula | | | | | | | |
| | | (2) | | | | | | | |
| 4 | Ab5D- | Compound | Ab5D | 5 U/mL | 15% | 40:1 | 40% | 0.2% | 5.63 |
| | CD-2 | of Formula | | | | | | | |
| | | (2) | | | | | | | |

### 3. Antigen Binding Capacity of the ADCs

The ADCs in the present example were tested for their affinities to human PD-L 1/B7-H1/CD274, and bispecific antibody-drug linker conjugates were further tested for their affinities towards human Trop-2/TACSTD2. Results are shown in Table 20.

TABLE 20
Affinities of the ADCs

| NO. | Ligand | Analyte | ka(1/Ms) | kd(1/s) | KD(M) |
|---|---|---|---|---|---|
| 1 | Ab3A-CD-1 | PD-L1/B7- | 6.688E+05 | 2.187E−04 | 3.270E−10 |
| | | H1/CD274 | | | |
| 2 | Ab3A-CD-2 | PD-L1/B7- | 6.810E+05 | 2.130E−04 | 3.127E−10 |
| | | H1/CD274 | | | |
| 3 | Ab3F-CD-1 | PD-L1/B7- | 7.272E+05 | 2.228E−04 | 3.063E−10 |
| | | H1/CD274 | | | |
| 4 | Ab3F-CD-2 | PD-L1/B7- | 7.315E+05 | 2.223E−04 | 3.039E−10 |
| | | H1/CD274 | | | |
| 5 | Ab5C-CD-1 | PD-L1/B7- | 8.666E+05 | 2.272E−04 | 2.622E−10 |
| | | H1/CD274 | | | |
| 6 | Ab5C-CD-2 | PD-L1/B7- | 8.926E+05 | 2.388E−04 | 2.676E−10 |
| | | H1/CD274 | | | |
| 7 | Ab5D-CD-1 | PD-L1/B7- | 8.326E+05 | 2.230E−04 | 2.679E−10 |
| | | H1/CD274 | | | |
| 8 | Ab5D-CD-2 | PD-L1/B7- | 8.419E+05 | 2.311E−04 | 2.745E−10 |
| | | H1/CD274 | | | |
| 9 | Ab2B | PD-L1/B7- | 7.055E+05 | 2.167E−04 | 3.072E−10 |
| | | H1/CD274 | | | |
| 10 | Ab5C-CD-1 | Trop- | 5.850E+04 | 1.151E−04 | 1.968E−09 |
| | | 2/TACSTD2 | | | |
| 11 | Ab5C-CD-2 | Trop- | 6.376E+03 | 1.040E−04 | 1.631E−08 |
| | | 2/TACSTD2 | | | |
| 12 | Ab5D-CD-1 | Trop- | 3.397E+04 | 1.727E−04 | 5.085E−09 |
| | | 2/TACSTD2 | | | |
| 13 | Ab5D-CD-2 | Trop- | 3.770E+04 | 1.642E−04 | 4.355E−09 |
| | | 2/TACSTD2 | | | |

### Example 7. Synthesis and Characterization of Antibody Oligonucleotide Conjugate (AOC)

### 1. Non-Covalent Antibody-CpG ODN Conjugation

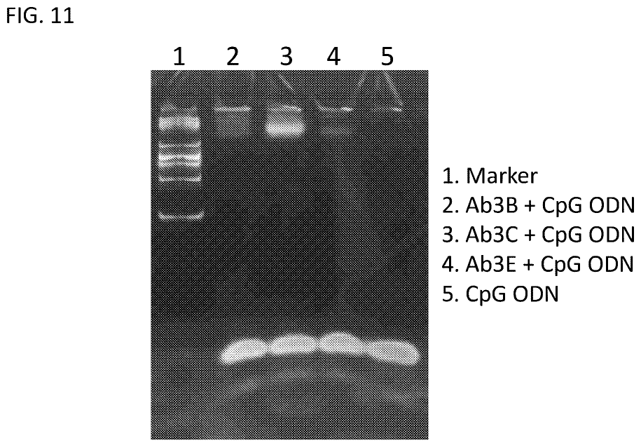

CpG ODN of SEQ ID NO: 67 was conjugated non-covalently to anti-PD-L1 antibody Ab3B (heavy chain SEQ ID NO: 106 and light chain SEQ ID NO:107), Ab3C (heavy chain SEQ ID NO: 108 and light chain SEQ ID NO: 109), and Ab3E (heavy chain SEQ ID NO: 110 and light chain SEQ ID NO:111). The design of the conjugation experiments is shown in Table 21. Reaction mixtures were incubated at room temperature for 16 hours, and drug-to-antibody ratio (DAR) of the conjugation product was determined by gel shift assay. The results are shown in FIG. 11.

TABLE 21
Experimental design

| | | Molar ratio | C_{mAb} | C_{CpG} | Time |
|---|---|---|---|---|---|
| mAb | CpG ODN | (mAb:CpG) | (mg/mL | mg/mL) | (h) |
| Ab3B | SEQ ID NO: 67 | 1:5 | 0.71 | 0.21 | 2.5 |
| Ab3C | SEQ ID NO: 67 | 1:5 | 0.71 | 0.21 | 2.5 |
| Ab3E | SEQ ID NO: 67 | 1:5 | 0.71 | 0.21 | 2.5 |

FIG. 11 shows that SEQ ID NO: 67 non-covalently bound to the antibodies effectively. The order of binding efficiency was Ab3C>Ab3B>Ab3E. Additionally, when the molar ratio of mAb:CpG was above 2:1, all CpG ODN was bound to Ab3C.

### 2. Covalent Antibody-CpG ODN Conjugation

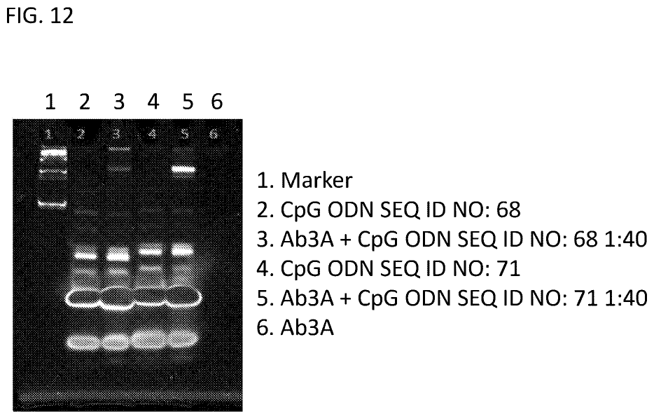

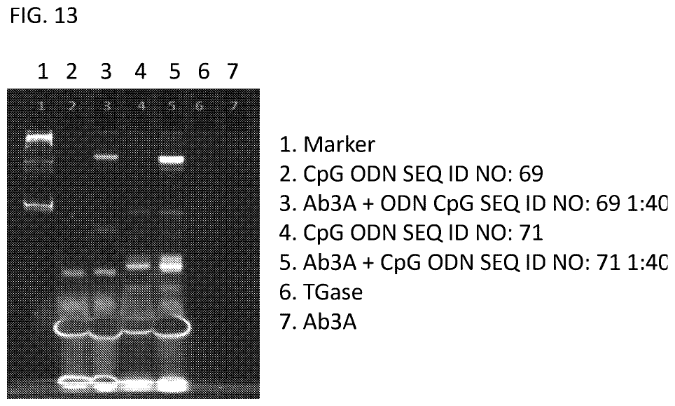

CpG ODN-linkers comprising CpG sequences of SEQ ID NOs: 68, 69 and 71 were conjugated covalently to Ab3A (heavy chain SEQ ID NO:104, light chain SEQ ID NO:105) by TGase, respectively. CpG ODN-linkers were mixed with Ab3A at 40:1 molar ratio. TGase was added to the reaction mixtures to a final concentration of 5 U/mL, and the reaction mixtures were incubated at room temperature for 16 hours. Conjugation products were analyzed by gel shift assay. The results are shown in FIGS. 12 and 13.

FIGS. 12 and 13 show that TGase was able to catalyze the conjugation between CpG ODN-linkers and antibodies with TGase substrate sequences. CpG ODN-linkers comprising a sequence of SEQ ID NO: 71 exhibited the highest conjugation efficiency, followed by CpG ODNs of SEQ ID NOs: 68 and 69. These results suggested that the length of the linker affected conjugation efficiency, with the longest of the three having the highest coupling efficiency.

### Example 8. Immune Checkpoint Bioassays for PD-1/PD-L1 Blockade

A luciferase reporter assay was used for mechanistic analysis of blocking PD-1/PD-L1 on the activation of the NFAT pathway. The bioassay consists of two genetically engineered cell lines: PD-1 effector cells, Jurkat T cells expressing human PD-1 and a luciferase reporter gene under the control of the NFAT promoter (NFAT-Luc2); PD-L1 aAPC/293T-OS8 Cells, 293T-OS8 cells expressing human PD-L1. Briefly, Jurkat-NFAT-Luc2-PD1 stable effector cells and the 293T-OS8-PD-L1 stable cell line were co-cultured at a ratio of 1:1 in the presence of serially diluted antibodies in triplicate for 6 h at 37° C., 5% CO₂. Luminescence was measured using ONE-Step™ Luciferase Assay System (BPS Bioscience).

### Results and Conclusion

TABLE 22
IC50 summary of PD-1/PD-L1 Immunoblockade reporter assay

| Drug name | IC₅₀ (nM) |
|---|---|
| Ab2B | 0.456 nM |
| Ab3A | 0.387 nM |
| Ab5C | 0.593 nM |
| Ab5D | 0.497 nM |
| Atezolizumab | 0.731 nM |

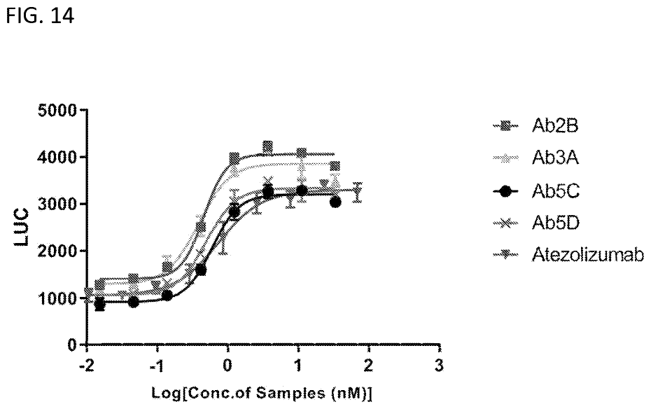

IC₅₀ values obtained from PD-1/PD-L1 Immunoblockade reporter assay are summarized in Table 22. The dose response curves of drugs are shown in FIG. 14. The results revealed that fusion antibody Ab3A, bi-specific antibody Ab5C and Ab5D, all constructed based on antibody Ab2B, have similar Immunoblockade potency to Ab2B, which are also similar to the reference antibody Atezolizumab.

### Example 9. In Vitro Cytotoxicity Study

### Cell Line Construction and Culture Conditions

Human colorectal adenocarcinoma HCT15 cell line stably expressing exogenous human PDL1 gene was generated using lentiviral plasmid with human PDL1 gene (NM_014143) sequence. The cell line was named as HCT15-hPDL1. Cells were cultured at 37° C. with 5% CO2 using RPMI1640 medium supplemented with 10% FBS, plus 1 μg/ml of puromycin.

### Cytotoxicity Study

The in vitro anti-tumor activity was determined by CellTiter-Glo® Luminescent Cell Viability Assay (Promega) according to the manufacturer's instructions. Briefly, cells were seeded into 96-well plates at 2×10³ cells per well in 100 μL complete medium, then incubated at 37° C. with 5% CO₂ overnight. Untreated cells served as control. ADCs, antibodies, or payloads in 100 μL medium were added in duplicate at various concentrations with a 2-fold dilution series starting with concentrations of 1 uM, respectively. Cells were exposed to test articles for 5 days. Absorbance was measured at 450 nm by a microplate reader. The cell survival rate (%) was calculated using the following formula: sample/control×100%. Dose-response curves were generated and the 50% inhibitory concentration (IC₅₀) was calculated by a non-linear regression analysis.

### Results and Conclusion

TABLE 23
IC50 summary of cytotoxicity study using HCT15-hPDL1 cells

| | | HCT15-hPDL1 |
|---|---|---|
| | | cell |
| Drug | Name | IC₅₀(nM) |
| Compound | SN38 | 11.18 |
| | Compound of Formula (1) | >300 |
| | Compound of Formula (2) | 38.13 |
| Antibody | Ab2B | >300 |
| | Ab3A | >300 |
| | Ab3F | >300 |
| ADC | Ab3A-CD-1 | >300 |
| | Ab3A-CD-2 | 16.49 |
| | Ab3F-CD-1 | >300 |
| | Ab3F-CD-2 | 29.51 |

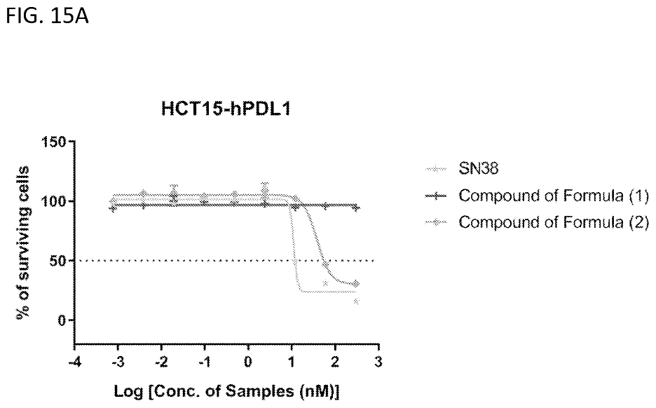

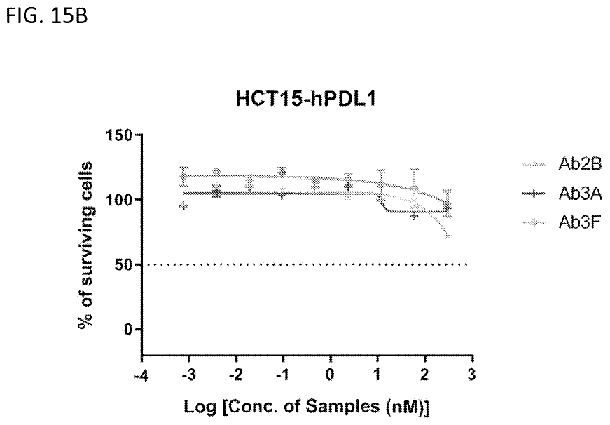

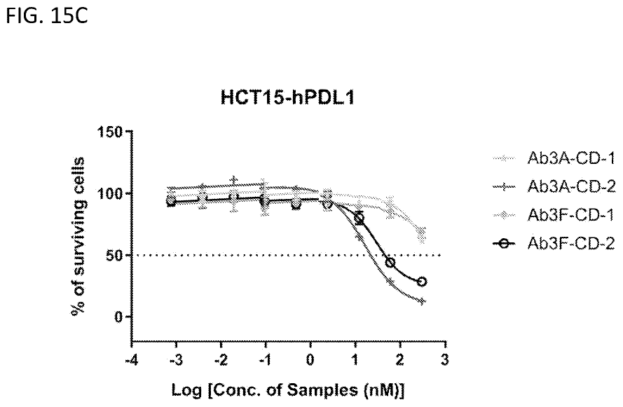

IC₅₀ values obtained from in vitro cytotoxicity study using HCT15-hPDL1 cells are summarized in Table 23. The dose response cytotoxicity curves of drugs are shown in FIG. 15. Ab3A-CD-1 is a conjugate of antibody Ab3A and drug-linker Compound of Formula (1), Ab3A-CD-2 is a conjugate of antibody Ab3A and drug-linker Compound of Formula (2), Ab3F-CD-1 is a conjugate of antibody Ab3F and drug-linker Compound of Formula (1), and Ab3F-CD-2 is a conjugate of antibody Ab3F and drug-linker Compound of Formula (2).

The results of in vitro cytotoxicity using HCT15-hPDL1 cells revealed that the cytotoxic potency of SN38 linked Compound of Formula (1) and Compound of Formula (2) was significantly lower than that of SN38. The cytotoxic potency of Compound of Formula (2) was better than that of Compound of Formula (1) (as shown in FIG. 15A). No cytotoxicity was found with antibody Ab2B, Ab3A and Ab3F (as shown in FIG. 15B). The cytotoxic potency of Ab3A-CD-2 and Ab3F-CD-2 containing Compound of Formula (2) was significantly better than that of Ab3A-CD-1 and Ab3F-CD-1 containing Compound of Formula (1) (as shown in FIG. 15C).

### Example 10. In Vivo Therapeutic Efficacy Study Using MDA-MB-231 CDX Model

### Cell Lines

Human breast adenocarcinoma cell line MDA-MB-231 from ATCC were cultured at 37° C. without CO₂ using Leibovitz's L-15 medium supplemented with 10% FBS.

### Animals

The severely immunodeficient M-NSG mice were housed and maintained in the specific pathogen free (SPF) grade of animal care facility at a CRO company (Shanghai, China). 6-8 weeks old mice at the experimental initiation were maintained with standard laboratory chow and water ad libitum. All animal experiments were approved and performed according to the company's Institutional Animal Care Guidelines.

### In Vivo Therapeutic Efficacy Study

For the cell line-derived xenograft (CDX) model, 1×10⁷ MDA-MB-231 cells suspended in 100 μL of PBS with 30% Matrigel were injected subcutaneously into the right flank of M-NSG female mice. When the tumor volume reached 80-200 mm³, the mice were grouped with similar average tumor volume and body weight and injected intraperitoneally with samples at doses described in the Table 24. The day of grouping and dosing was denoted as day 0. Tumor volumes were measured twice a week over the entire duration of the studies. The tumor volumes were determined according to the formula: tumor volume (mm³)=(length×width²)×0.5.

Data of all experiments were presented as mean values±standard deviations (SD) by Graphpad Prism 8.0 software. The statistical significance between two groups was determined using two-way ANOVA followed by Student's t-test. The p-values less than 0.05 were considered statistically significant.

TABLE 24
Dosage summary sheet of MDA-MB-231 CDX model

| | | | | | | Total |
|---|---|---|---|---|---|---|
| | | | | | | Number |
| | | Number | Dose | Injection | Dosing | of |
| Group | Drug Tested | of Mice | (mg/kg) | Route | Frequency | Dosing |
| 1 | Vehicle | 10 | — | i.p. | BIW | 6 |
| 2 | Ab2B | 10 | 15 | i.p. | BIW | 6 |
| 3 | Ab3A-CD-1 | 10 | 15 | i.p. | BIW | 6 |
| 4 | Ab3A-CD-2 | 10 | 15 | i.p. | BIW | 6 |
| 5 | Ab3F-CD-1 | 10 | 15 | i.p. | BIW | 6 |
| 6 | Ab3F-CD-2 | 10 | 15 | i.p. | BIW | 6 |

### Results and Conclusion

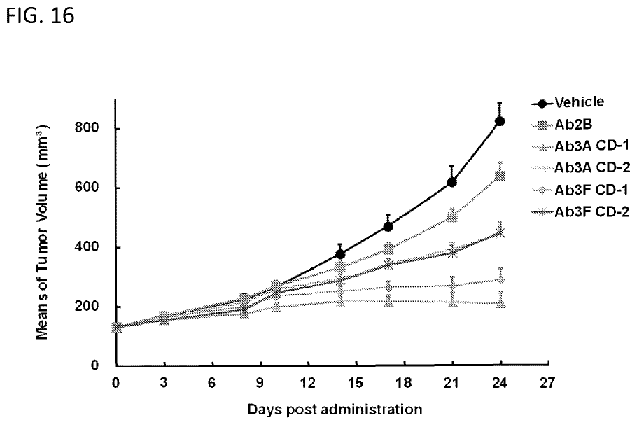

As shown in FIG. 16, the in vivo therapeutic efficacy study has revealed that in MDA-MB-231 CDX model, all four ADCs have better tumor inhibition effects compared to antibody Ab2B. When conjugated with the same Compound of Formula (1) or Compound of Formula (2), antibody Ab3A and Ab3F showed similar tumor inhibition effect. Ab3A-CD-1 and Ab3F-CD-1 containing Compound of Formula (1) demonstrated better tumor inhibition effects in vivo compared to Ab3A-CD-2 and Ab3F-CD-2 containing Compound of Formula (2). Therefore, Compound of Formula (1) was further studied as a model drug-linker compound in ADCs in the subsequent in vitro and in vivo efficacy studies.

### Example 11. In Vitro Cytotoxicity Study of Bi-Specific ADC

### Cell Line Construction and Culture Conditions

Human prostate carcinoma PC3 cell line stably expressing exogenous human PDL1 gene and human TROP2 gene was generated using lentiviral plasmids with human PDL1 gene (NM_014143) and human TROP2 (NM_002353.2) gene sequences. The cell line was named as PC3-hPDL1-hTROP2. Cells were cultured at 37° C. with 5% CO₂ using Ham's F12K medium supplemented with 10% FBS, plus 1 μg/ml of puromycin and 100 ug/ml of hygromycin.

Human colorectal adenocarcinoma HCT15 cell line stably expressing exogenous human PDL1 gene and human TROP2 gene was generated using lentiviral plasmids with human PDL1 gene (NM_014143) and human TROP2 (NM_002353.2) gene sequences. The cell line was named as HCT15-hPDL1-hTROP2. Cells were cultured at 37° C. with 5% CO₂ using RPMI1640 medium supplemented with 10% FBS, plus 1 μg/ml of puromycin and 100 ug/ml of hygromycin.

### Cytotoxicity Study

The in vitro anti-tumor activity was determined by CellTiter-Glo® Luminescent Cell Viability Assay (Promega) according to the manufacturer's instructions. Briefly, cells were seeded into 96-well plates at 2×10³ cells per well in 100 μL complete medium, then incubated at 37° C. with 5% CO₂ overnight. Untreated cells served as control. ADCs, antibodies, or payloads in 100 μL medium were added in duplicate at various concentrations with a 2-fold dilution series starting with concentrations of 1 uM, respectively. Cells were exposed to test articles for 5 days. Absorbance was measured at 450 nm by a microplate reader. The cell survival rate (%) was calculated using the following formula: sample/control×100%. Dose-response curves were generated and the 50% inhibitory concentration (IC₅₀) was calculated by a non-linear regression analysis.

### Results and Conclusion

TABLE 25
IC50 summary of cytotoxicity study
using PC3-hPDL1-hTROP2 cells

| | | | Maximum |
|---|---|---|---|
| | | PC3-hPDL1-hTROP2 | Inhibitory |
| | | cell | Rate |
| Drug | Name | IC₅₀(nM) | (%) |
| Compound | SN38 | 8.685 | |
| | Compound of | >250 | |
| | Formula (1) | | |
| | Compound of | 53.13 | |
| | Formula (2) | | |
| Antibody | Ab2B | >250 | |
| | Ab5C | >250 | |
| | Ab5D | >250 | |
| ADC | Ab3A-CD-1 | 34.69 | 72.2 |
| | Ab5C-CD-1 | 10.15 | 99.4 |
| | Ab5D-CD-1 | 5.983 | 99.0 |

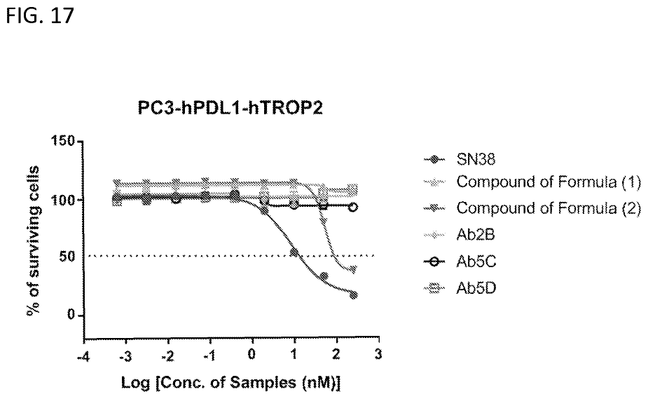

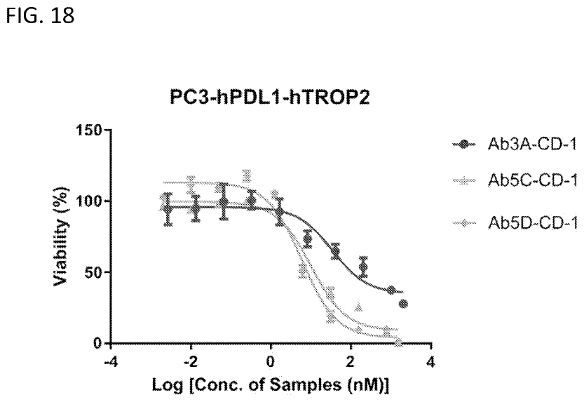

IC₅₀ values obtained from in vitro cytotoxicity study using PC3-hPDL1-hTROP2 cells are summarized in Table 25. The dose response cytotoxicity curves of drugs are shown in FIGS. 17 and 18. Ab3A-CD-1 is a conjugate of antibody Ab3A and drug-linker Compound of Formula (1), Ab5C-CD-1 is a conjugate of bi-specific antibody Ab5C with no uPA cleavage site at Fc region and drug-linker Compound of Formula (1), and Ab5D-CD-1 is a conjugate of bi-specific antibody Ab5D containing uPA cleavage sites at Fc region and drug-linker Compound of Formula (1).

The results of in vitro cytotoxicity using PC3-hPDL1-hTROP2 cells revealed that the cytotoxic potency of SN38 linked Compound of Formula (1) and Compound of Formula (2) was significantly lower than that of SN38. The cytotoxic potency of Compound of Formula (2) was better than that of Compound of Formula (1). No cytotoxicity was found with antibody Ab2B, bi-specific antibody Ab5C and Ab5D. The cytotoxic potency of Ab5C-CD-1 and Ab5D-CD-1 is significantly greater than that of Ab3A-CD-1. Ab5D-CD-1 has better cytotoxic potency than Ab5C-CD-1, indicating uPA cleavage sites in Fc region of bi-specific antibody improves in vitro cytotoxicity.

TABLE 26
IC50 summary of cytotoxicity assay using HCT15-hPDL1-hTROP2 cells

| | | HCT15-hPDL1-hTROP2 | Maximum Inhibitory |
|---|---|---|---|
| Drug | Name | cell IC₅₀(nM) | Rate (%) |
| Compound | SN38 | 9.57 | >99.9 |
| ADC | Ab3A-CD-1 | 289.50 | 95.1 |
| | Ab5C-CD-1 | 19.01 | >99.9 |
| | Ab5D-CD-1 | 7.18 | 96.1 |

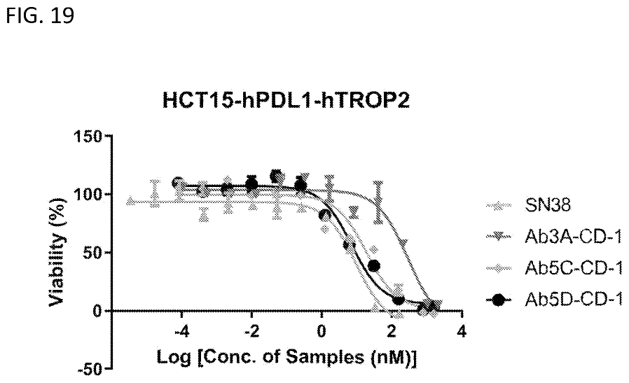

IC₅₀ values obtained from in vitro cytotoxicity study using HCT15-hPDL1-hTROP2 cells are summarized in Table 26. The dose response cytotoxicity curves of drugs are shown in FIG. 19. The in vitro cytotoxicity results demonstrate that the cytotoxic potency of Ab5C-CD-1 and Ab5D-CD-1 is significantly greater than that of Ab3A-CD-1. Ab5D-CD-1 has better cytotoxic potency than Ab5C-CD-1, indicating uPA cleavage site in Fc region of bi-specific antibody improves in vitro cytotoxicity.

### Example 12. In Vivo Therapeutic Efficacy Study Using MC38-hPDL1-hTROP2 Mouse Syngeneic Tumor Model

### Cell Lines

Murine colon adenocarcinoma MC38 cell line stably expressing exogenous human PDL1 gene and human TROP2 gene was generated using lentiviral plasmids with human PDL1 gene (NM_014143) and human TROP2 (NM_002353.2) gene sequences. The cell line was named as MC38-hPDL1-hTROP2. Cells were cultured at 37° C. with 5% CO₂ using DMEM medium supplemented with 10% FBS, 1% glutamine and 1% of penicillin/streptomycin.

### Animals

Transgenic C57BL/6J mice system expressing human PD-L1, C57BL/6-hPDL1 mice were housed and maintained in the specific pathogen free (SPF) grade of animal care facility at a CRO company (Shanghai, China). 6-8 weeks old mice at the experimental initiation were maintained with standard laboratory chow and water ad libitum. All animal experiments were approved and performed according to the company's Institutional Animal Care Guidelines.

### In Vivo Therapeutic Efficacy Study

For the syngeneic tumor mouse mode, 5×10⁶ MC38-hPDL1-hTROP2 cells suspended in 100 μL of PBS were injected subcutaneously into the right flank of C57BL/6-hPDL1 female mice. When the tumor volume reached 100-150 mm³, the mice were grouped with similar average tumor volume and body weight and injected intravenously with samples at doses described in the Table 27. The day of grouping and dosing was denoted as day 0. Tumor volumes were measured twice a week over the entire duration of the studies. The tumor volumes were determined according to the formula: tumor volume (mm³)=(length×width²)×0.5.

Data of all experiments were presented as mean values±standard deviations (SD) by Graphpad Prism 8.0 software. The statistical significance between two groups was determined using two-way ANOVA followed by Student's t-test. The p-values less than 0.05 were considered statistically significant.

TABLE 27
Dosage summary Sheet of MC38-hPDL1-hTROP2 mouse
syngeneic tumor model

| | | | | | | Total |
|---|---|---|---|---|---|---|
| | | | | | | Number |
| | | Number | Dose | Injection | Dosing | of |
| Group | Drug Tested | of Mice | (mg/kg) | Route | Frequency | Dosing |
| 1 | Vehicle | 8 | N/A | i.v. | BIW | 2 |
| 2 | Ab2B | 8 | 10 | i.v. | BIW | 2 |
| 3 | Ab6A-ADC | 8 | 10 | i.v. | BIW | 2 |
| 4 | Ab5C-CD-1 | 8 | 15 | i.v. | BIW | 2 |
| 5 | Ab5D-CD-1 | 8 | 15 | i.v. | BIW | 2 |
| 6 | Ab5C-CD-1 | 8 | 35 | i.v. | QW | 1 |
| 7 | Ab5D-CD-1 | 8 | 35 | i.v. | QW | 1 |

### Results and Conclusion

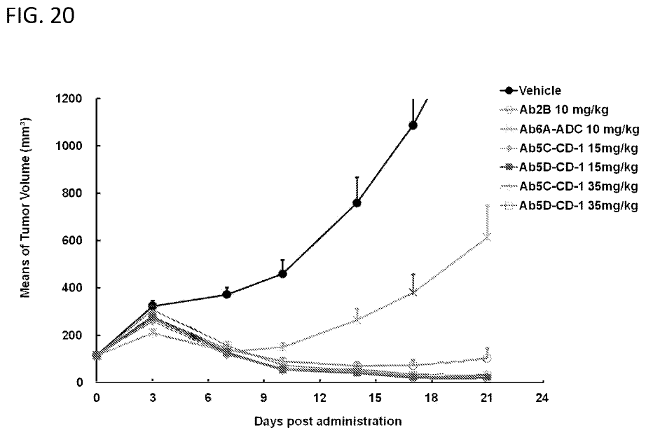

The results of in vivo therapeutic efficacy study using MC38-hPDL1-hTROP2 mouse syngeneic tumor model are shown in FIG. 20. Ab6A-ADC was as a reference ADC drug, a conjugate of anti-TROP2 hRS7 antibody and drug-linker CL2A-SN38.

The in vivo therapeutic efficacy study has shown that all tested drugs, including antibody Ab2B, ADC Ab6A ADC, Ab5C-CD-1 and Ab5D-CD-1, have better tumor inhibition effects than vehicle. Bi-specific antibody ADCs, Ab5C-CD-1 and Ab5D-CD-1, have better tumor inhibition effects than antibody Ab2B and ADC Ab6A ADC. At different dosing regimen, 15 mg/kg twice per week or 35 mg/kg once per week, Ab5C-CD-1 and Ab5D-CD-1 have no significant difference in tumor inhibition within the period studied.

### Example 13. In Vivo Therapeutic Efficacy Study Using PBMCs—PC3-hPDL1-hTROP2 CDX Model

### Cell Lines

Human prostate carcinoma PC3 cell line stably expressing exogenous human PDL1 gene and human TROP2 gene was generated using lentiviral plasmids with human PDL1 gene (NM_014143) and human TROP2 (NM_002353.2) gene sequences. The cell line was named as PC3-hPDL1-hTROP2. Cells were cultured at 37° C. with 5% CO₂ using Ham's F12K medium supplemented with 10% FBS, plus 1 μg/ml of puromycin and 100 ug/ml of hygromycin.

### Animals

The severely immunodeficient M-NSG mice were housed and maintained in the specific pathogen free (SPF) grade of animal care facility at a CRO company (Shanghai, China). 6-8 weeks old mice at the experimental initiation were maintained with standard laboratory chow and water ad libitum. All animal experiments were approved and performed according to the company's Institutional Animal Care Guidelines.

### In Vivo Therapeutic Efficacy Study

For the peripheral blood mononuclear cells and cell line-derived xenografts (PBMCs-CDX) model, 1×10⁷ PC3-hPDL1-hTROP2 cells suspended in 100 μL of PBS with 30% Matrigel were injected subcutaneously into the right flank of M-NSG female mice, followed by injection of 5×10⁶ human PBMCs suspended in 200 μL of PBS intravenously. When the tumor volume reached 80-160 mm³, the mice were grouped with similar average tumor volume and body weight and injected intravenously with samples at doses described in the Table 28. The day of grouping and dosing was denoted as day 0. Tumor volumes were measured twice a week over the entire duration of the studies. The tumor volumes were determined according to the formula: tumor volume (mm³)=(length×width²)×0.5.

Data of all experiments were presented as mean values±standard deviations (SD) by Graphpad Prism 8.0 software. The statistical significance between two groups was determined using two-way ANOVA followed by Student's t-test. The p-values less than 0.05 were considered statistically significant.

TABLE 28
Dosage summary sheet of PBMCs-PC3-hPDL1-hTROP2 CDX model

| | | | | | | Total |
|---|---|---|---|---|---|---|
| | | | | | | Number |
| | | Number | Dose | Injection | Dosing | of |
| Group | Drug Tested | of Mice | (mg/kg) | Route | Frequency | Dosing |
| 1 | Ab2B | 8 | 10 | i.v. | BIW | 3 |
| 2 | Ab5C-CD-1 | 8 | 15 | i.v. | BIW | 3 |
| 3 | Ab5D-CD-1 | 8 | 15 | i.v. | BIW | 3 |
| 4 | Ab5C-CD-1 | 8 | 35 | i.v. | QW | 2 |
| 5 | Ab5D-CD-1 | 8 | 35 | i.v. | QW | 2 |

### Results and Conclusion

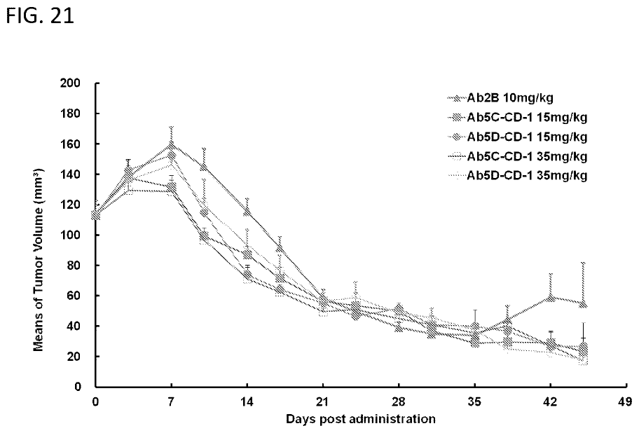

The results of in vivo therapeutic efficacy study using PBMCs-PC3-hPDL1-hTROP2 CDX model are shown in FIG. 21. The in vivo therapeutic efficacy study has shown that Ab5C-CD-1 and Ab5D-CD-1 have better tumor inhibition effects than antibody Ab2B. At different dosing regimen, 15 mg/kg twice per week or 35 mg/kg once per week, Ab5C-CD-1 and Ab5D-CD-1 have no significant difference in tumor inhibition within the period studied. However, at 35 mg/kg dosing regimen, three mice (3/8) were died on 9^{th}, 11^{th}, and 15^{th} day after initial administration of Ab5C-CD-1, no mouse (0/8) was died after administration of Ab5D-CD-1 within the same period studied, indicating Ab5D-CD-1 containing uPA cleavage sites in Fc region has better safety than Ab5C-CD-1 without uPA cleavage site in Fc region.

### Example 14. In Vivo Therapeutic Efficacy Study Using HCT15-hPDL1-hTROP2 CDX Model

### Cell Lines

Human colorectal adenocarcinoma HCT15 cell line stably expressing exogenous human PDL1 gene and human TROP2 gene was generated using lentiviral plasmids with human PDL1 gene (NM_014143) and human TROP2 (NM_002353.2) gene sequences. The cell line was named as HCT15-hPDL1-hTROP2. Cells were cultured at 37° C. with 5% CO₂ using RPMI1640 medium supplemented with 10% FBS, plus 1 μg/ml of puromycin and 100 ug/ml of hygromycin.

### Animals

The severely immunodeficient B-NDG mice were housed and maintained in the specific pathogen free (SPF) grade of animal care facility at a CRO company (Beijing, China). 8-10 weeks old mice at the experimental initiation were maintained with standard laboratory chow and water ad libitum. All animal experiments were approved and performed according to the company's Institutional Animal Care Guidelines.

### In Vivo Therapeutic Efficacy Study

For the cell line-derived xenograft (CDX) model, 1×10⁶ HCT15-hPDL1-hTROP2 cells suspended in 100 μL of PBS were injected subcutaneously into the right flank of B-NDG female mice. When the tumor volume reached 80-120 mm³, the mice were grouped with similar average tumor volume and body weight and injected intravenously with samples at doses described in the Table 29. The day of grouping and dosing was denoted as day 0. Tumor volumes were measured twice a week over the entire duration of the studies. The tumor volumes were determined according to the formula: tumor volume (mm³)=(length×width²)×0.5.

Data of all experiments were presented as mean values±standard deviations (SD) by Graphpad Prism 8.0 software. The statistical significance between two groups was determined using two-way ANOVA followed by Student's t-test. The p-values less than 0.05 were considered statistically significant.

TABLE 29
Dosage summary sheet of HCT15-hPDL1-hTROP2 CDX model

| | | | | | | | | Total |
|---|---|---|---|---|---|---|---|---|
| | | | | | | | | Number |
| | | Number | Injection | Dose | Dosing | Dose | Dosing | of |
| Group | Drug Tested | of Mice | Route | (mg/Kg) | Frequency | (mg/Kg) | Frequency | Dosing |
| 1 | Ab5C-CD-1 | 8 | i.v | 10 | QW*2 | 15 | QW*7 | 9 |
| 2 | Ab5D-CD-1 | 8 | i.v | 10 | QW*2 | 15 | QW*7 | 9 |

### Results and Conclusion

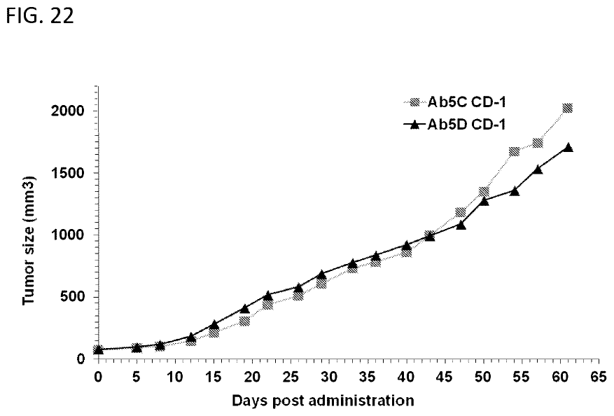

The results of in vivo therapeutic efficacy study using HCT15-hPDL1-hTROP2 CDX model are shown in FIG. 22. The in vivo therapeutic efficacy study has shown that Ab5D-CD-1 has better tumor inhibition effect in long-term than Ab5C-CD-1, indicating uPA cleavage site in Fc region of bi-specific antibody also improves in vivo efficacy.

### Example 15. In Vitro TLR9 Activation by CpG ODN-Linkers

### Cell Culture

HEK-Blue™ hTLR9 cells are engineered HEK293 cells that stably co-express the human TLR9 and an NF-kB-inducible SEAP (secreted embryonic alkaline phosphatase) reporter gene. The HEK-Blue™ Detection assays allow the detection of SEAP production following TLR9 activation by reading the optical density (OD) at 655 nm.

HEK-Blue™ hTLR9 cells were obtained from InvivoGen. The cells were cultured in DMEM medium supplemented with 4.5 g/l glucose, 10% FBS, 2 mM L-glutamine, 100 U/ml penicillin, 100 μg/ml streptomycin, 100 μg/ml Normocin™, 10 mg/ml of Blasticidin and 100 mg/ml of Zeocin™ at 37° C. in humidified air containing 5% CO₂ as per the manufacturer's instructions.

Response of HEK-Blue™ hTLR9 Cells to CpG ODN-Linkers

The HEK-Blue hTLR9 cells were seeded onto 96-well plates at a density of 2×10⁵ cells/well in HEK-Blue™ Detection. HEK-Blue™ Detection is a cell culture medium that allows the detection of SEAP as the reporter protein is secreted by the cells. HEK-Blue™ hTLR9 cells were stimulated with CpG ODN CpGB, CpG ODN-Linker CpGC, CpGD, CpGE, and CpGF at a final concentration of 0.6 mg/mL. After 16 hours of incubation, the optical density (OD) of the samples was measured at a wavelength of 655 nm using a microplate reader. Data were normalized with untreated cells as zero. Each data point represented the average and standard deviation of two replicates.

### Results and Conclusion

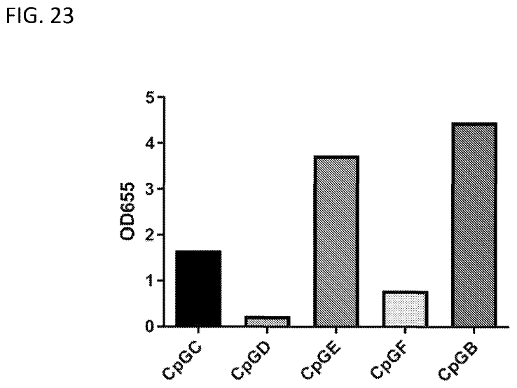

The response of HEK-Blue hTLR9-hPDL1 cells to different CpG ODN-Linkers is shown in FIG. 23. At the same concentration of 0.6 mg/ml, CpG ODN-Linker CpGE and CpG ODN CpGB had similar in vitro potency, better than CpG ODN-Linker CpGC, CpGD, and CpGF.

### Example 16. In Vitro TLR9 Activation by Antibody-Oligonucleotide Conjugate (AOC)

### Cell Line Construction and Culture Conditions

The HEK-Blue™ hTLR9 cell line stably expressing exogenous human PDL1 gene was generated using lentiviral plasmid with human PDL1 gene (NM_014143) sequence. The constructed cell line was named as HEK-Blue hTLR9-hPDL1. Cells were cultured at 37° C. with 5% CO₂ using DMEM medium supplemented with 4.5 g/l glucose, 10% FBS, 2 mM L-glutamine, 100 U/ml penicillin, 100 μg/ml streptomycin, 1 μg/ml of puromycin and 10 ug/ml of blasticidin.

Response of HEK-Blue hTLR9-hPDL1 cells to Antibody-Oligonucleotide Conjugate (AOC)

The HEK-Blue hTLR9-hPDL1 cells were seeded onto 96-well plates at a density of 1×10⁵ cells/well in HEK-Blue™ Detection. HEK-Blue™ Detection is a cell culture medium that allows the detection of SEAP as the reporter protein is secreted by the cells. HEK-Blue hTLR9-hPDL1 cells were then treated with different concentrations of Ab3A AOC as indicated, with CpG ODN CpGB and antibody Ab2B as positive and negative controls respectively. After 18 h incubation, the optical density (OD) of the samples was measured at a wavelength of 655 nm using a microplate reader. Data were normalized with untreated cells as zero. Each data point represents the average and standard deviation of two replicates.

### Results and Conclusion

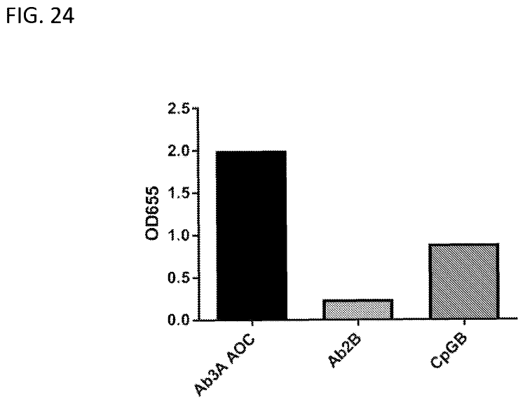

The response of HEK-Blue hTLR9-hPDL1 cells to AOC is shown in FIG. 24. Ab3A AOC is a conjugate of antibody Ab3A with CpG ODN-Linker CpGE. The in vitro TLR9 activation study results reveal that Ab3A AOC has better potency than antibody Ab2B and CpG ODN CpGB, respectively, at the same concentration of 6 μM.

### Example 17. In Vivo Therapeutic Efficacy Study Using MC38-hPDL1 Mouse Syngeneic Tumor Model

### Cell Lines

Murine colon adenocarcinoma MC38 cell line stably expressing exogenous human PDL1 gene was generated using lentiviral plasmid with human PDL1 gene (NM_014143) sequence. The cell line was named as MC38-hPDL1. Cells were cultured at 37° C. with 5% CO₂ using DMEM medium supplemented with 10% FBS, 1% glutamine and 1% of penicillin/streptomycin.

### Animals

Transgenic C57BL/6J mice system expressing human PD-L1, C57BL/6-hPDL1 mice were housed and maintained in the specific pathogen free (SPF) grade of animal care facility at a CRO company (Shanghai, China). 6-8 weeks old mice at the experimental initiation were maintained with standard laboratory chow and water ad libitum. All animal experiments were approved and performed according to the company's Institutional Animal Care Guidelines.

### In Vivo Therapeutic Efficacy Study

For the syngeneic tumor mouse mode, 1×10⁶ MC38-hPDL1 cells suspended in 100 μL of PBS were injected subcutaneously into the right flank of C57BL/6-hPDL1 female mice. When the tumor volume reached 100-150 mm³, the mice were grouped with similar average tumor volume and body weight and injected intravenously with samples at doses described in the Table 30. The day of grouping and dosing was denoted as day 0. Tumor volumes were measured twice a week over the entire duration of the studies. The tumor volumes were determined according to the formula: tumor volume (mm³)=(length×width²)×0.5.

Data of all experiments were presented as mean values±standard deviations (SD) by Graphpad Prism 8.0 software. The statistical significance between two groups was determined using two-way ANOVA followed by Student's t-test. The p-values less than 0.05 were considered statistically significant.

TABLE 30
Dosage summary sheet of MC38-hPDL1 mouse syngeneic
tumor model

| | | | | | | Total |
|---|---|---|---|---|---|---|
| | | | | | | Number |
| | Drug | Number | Dose | Injection | Dosing | of |
| Group | Tested | of Mice | (mg/kg) | Route | Frequency | Dosing |
| 1 | Vehicle | 8 | N/A | i.v. | BIW + QW | 3 |
| 2 | Ab2B | 8 | 13 | i.v. | BIW + QW | 3 |
| 3 | Ab3A | 8 | 15 | i.v. | BIW + QW | 3 |
| | AOC | | | | | |

### Results and Conclusion

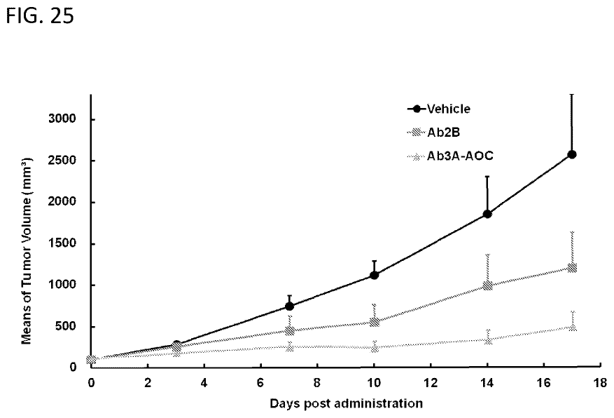

The results of in vivo therapeutic efficacy study using MC38-hPDL1 mouse syngeneic tumor model are shown in FIG. 25. Antibody Ab2B and AOC Ab3A AOC have demonstrated better tumor inhibition effects compared to the vehicle. AOC Ab3A AOC showed better tumor inhibition compared to antibody Ab2B.

1-58. (canceled)

59. A targeting conjugate comprising a targeting moiety and an effector molecule, wherein the targeting moiety specifically binds to a target molecule, wherein the effector molecule is conjugated to the targeting moiety via a conjugation site, and wherein the effector molecule can be released from the targeting conjugate via cleavage.

60. The targeting conjugate of claim 59, wherein the targeting conjugate comprises the following structure: 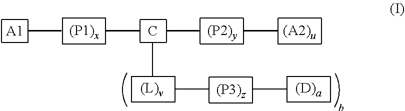
wherein:
A1 is a first targeting moiety;
A2 is a second targeting moiety;
P1 is a first cleavage site;
P2 is a second cleavage site;
P3 is a third cleavage site;
C is the conjugation site;
L is a linker;
D is the effector molecule;
x=0 or 1;
y=0 or 1;
z=0 or 1;
v=0 or 1;
u=0 or 1;
a=1-20; and
b=1-20.

61. The targeting conjugate of claim 59, wherein the cleavage is triggered by a condition at a target site.

62. The targeting conjugate of claim 61, wherein
i) the condition at the target site is selected from the group consisting of: protease, pH change, redox change, hypoxia, oxidative stress, hyperthermia, and extracellular ATP concentration; and/or
ii) the target site is a site of a disease.

63. The targeting conjugate of claim 59, wherein the cleavage is by a protease.

64. The targeting conjugate of claim 63, wherein the protease is uPA, and wherein the cleavage site comprises the amino acid sequence of any of SEQ ID NOs: 50-55.

65. The targeting conjugate of claim 59, wherein the effector molecule is selected from the group consisting of a therapeutic agent, an oligonucleotide, and a detectable label.

66. The targeting conjugate of claim 59, wherein the targeting conjugate comprises:
i) a moiety having the structure of 5′Amino Modifier-Spacer-Pₕ-CpG ODN, or
ii) a moiety having the structure of 3′Amino Modifier-Spacer-Pₕ-CpG ODN;
wherein Spacer is (CH₂)ₙ-(PEG)ₘ, P is a cleavage site, and wherein h, n, and m are integers, h=0 or 1, n≥1, and m≥0.

67. The targeting conjugate of claim 60, wherein L is represented by the formula (Gly)ₙ-(PEG)ₘ-P-PAB-(DMAE)ₖ, (Gly)ₙ-(PEG)ₘ-VC-PAB-(DMAE)ₖ, or (Gly)ₙ-(PEG)ₘ-Val-Ala-PAB-(DMAE)ₖ; and wherein n, m, and k are integers, n≥1, m≥2, k is 0 or 1, and P is a cleavage site.

68. The targeting conjugate of claim 59, wherein the conjugation site is a transglutaminase conjugation site.

69. The targeting conjugate of claim 68, wherein:
i) the transglutaminase conjugation site comprises a plurality of glutamine-containing tags that are fused to each other in tandem; and/or
ii) the transglutaminase conjugation site comprises the amino acid sequence of any of SEQ ID NOs: 1-49, 145, and 153-155.

70. The targeting conjugate of claim 59, wherein the target molecule is an immune checkpoint molecule or a tumor antigen.

71. The targeting conjugate of claim 60, wherein A1 is a first targeting peptide, or a first antibody or antigen-binding fragment thereof recognizing a first target molecule, and wherein A2 is a second targeting peptide, or a second antibody or antigen-binding fragment thereof recognizing a second target molecule.

72. The targeting conjugate of claim 71, wherein
i) the first target molecule is a first immune checkpoint molecule, and the second target molecule is a second immune checkpoint molecule; or
ii) the first target molecule is a first tumor antigen, and the second target molecule is a second tumor antigen.

73. The targeting conjugate of claim 71, wherein
i) the first target molecule is an immune checkpoint molecule, and the second target molecule is a tumor antigen; or
ii) the first target molecule is a tumor antigen, and the second target molecule is an immune checkpoint molecule.

74. The targeting conjugate of claim 73, wherein the immune checkpoint molecule is PD-L1, and/or the tumor antigen is Trop-2.

75. The targeting conjugate of claim 60, wherein
i) u=1, A1 is a full-length anti-PD-L1 antibody, A2 is an anti-Trop-2 scFv, sdAb, or scFab; wherein the optional P1, C, and the optional P2 are fused to the C-terminus of one or both heavy chains of the full-length anti-PD-L1 antibody, and wherein A2 is fused to the C-terminus of the optional P2 at the C-terminus of the one or both heavy chains of the full-length anti-PD-L1 antibody; or
ii) u=0, A1 is a full-length anti-PD-L1 antibody, wherein the optional P1, C, and the optional P2 are fused to the C-terminus of the one or both heavy chains of the full-length anti-PD-L1 antibody.

76. A composition comprising a plurality of targeting conjugates of claim 59.

77. A method of treating a disease in an individual, comprising administering to the individual an effective amount of the composition of claim 76.

78. A method of making a targeting conjugate of claim 59, wherein the method comprises conjugating the effector molecule to the targeting moiety.

79. A method of diagnosing a disease in an individual, comprising administering to the individual an effective amount of the composition of claim 76, wherein the effector molecule is a detectable label, and wherein detection of the detectable label is indicative of the presence of the disease.

80. A targeting conjugate comprising a targeting moiety conjugated to a therapeutic agent, wherein the therapeutic agent comprises a structure selected from the group consisting of Formulae (1)-(7).
